(19) United States
(12) Patent Application Publication
(10) Pub. No.: US 2023/0226065 A1
(43) Pub. Date: Jul. 20, 2023

(54) ANTI-INFLAMMATORY COMPOSITIONS, METHODS AND USES THEREOF

(71) Applicants: Jacqueline Carol EVANS, Paengaroa, (NZ); Magaret BRIMBLE, Auckland (NZ); Rohith THOTA, Palmerston North (NZ); Dominic LOMIWES, Palmerston North (NZ); Odette Marianne SHAW, Palmerston North (NZ); COMVITA LIMITED, Paengaroa (NZ)

(72) Inventors: Bin LIN, Aucklana (NZ); Jonathan McDonald Counsell STEPHENS, Te Kauwhata (NZ); Kerry LOOMES, Auckland (NZ); Jacqueline Carol EVANS, Paengaroa (NZ); Margaret BRIMBLE, Auckland (NZ); Rohith THOTA, Palmerston North (NZ); Dominic LOMIWES, Palmerston North (NZ); Odette Marianne SHAW, Palmerston North (NZ)

(21) Appl. No.: 18/002,602

(22) PCT Filed: Jul. 2, 2021
(86) PCT No.: PCT/NZ2021/050103

§ 371 Date: Dec. 20, 2022

(30) Foreign Application Priority Data
Jul. 3, 2020 (NZ) 765957
Jul. 3, 2020 (WO) PCT/NZ2020/050065

(51) Int. Cl. A61K 31/519; A61K 35/644; A61K 36/61

(52) U.S. Cl. CPC A61K 31/519; A61K 35/644; A61K 36/61

(57) ABSTRACT

The disclosure relates to compositions comprising 3,6,7-trimethyllumazine. In particular, Manuka honey is an example of a composition comprising 3,6,7-trimethyllumazine. The composition is useful in preventing, ameliorating or treating TG2, JAK, and/or COX-2 associated conditions. These conditions include inflammation, pain, ulcers, Crohn's disease, reflux, gingivitis, schizophrenia, neurodegenerative disorders, Alzheimer's disease, Parkinson's disease, arthritis, cardiovascular diseases and cancers.

### RELATED APPLICATIONS

This application derives priority from New Zealand patent application number 765957 and PCT International patent application number PCT/NZ2020/050065, incorporated herein by reference.

### FIELD OF INVENTION

The invention relates to compositions comprising 3,6,7-trimethyllumazine, methods and uses thereof in preventing, ameliorating or treating inflammation and/or preventing, ameliorating or treating TG2, JAK, and/or COX-2 associated conditions. For example, conditions associated with inflammation, such as inflammation of the gastrointestinal tract and/or inflammatory conditions associated with the gastrointestinal tract.

### BACKGROUND OF THE INVENTION

Inflammation relating to the immune system can be beneficial, but this is not always the case. It is often considered to be a negative reaction or a reaction to be avoided; especially in the context of the gastrointestinal system.

Inflammation is implicated in a wide range of gastrointestinal disorders. In a healthy gut, the intestinal mucosa is in a state of controlled response regulated by an intricate balance of pro-inflammatory and anti-inflammatory cytokines and cells. Disruptions to this balance can culminate in a sustained activation of the immune/non-immune responses, resulting in active inflammation and tissue destruction. Failure to prevent or resolve inflammation adequately is implicated in the pathogenesis of several diseases of the gastrointestinal tract including gastric ulcers, inflammatory bowel disease (IBD), Crohn's disease and ulcerative colitis.

Depending on the severity, extent and medical goals of treatment, conventional medications for inflammatory conditions, such as sulfasalazine, mesalazine, corticosteroids, and methotrexate, are primarily used to modulate the immune and inflammatory responses. Limitations in both safety and efficacy encountered with current medical approaches for inflammatory conditions continue to drive the search for better and safer alternative therapeutic agents. Consumers are also looking more generally for natural ways to support their health and wellbeing.

Although the specific causes of inflammation are yet to be identified in many diseases, cytokine activation in the intestinal mucosal system is a key target for modulating inflammation in gut inflammatory diseases. Cyclooxygenase-2 (COX-2), Janus kinases (JAK) and Transglutaminase2 (TG2) are all known to be linked to IBD and many other inflammatory diseases.

Gastric ulcers are another common inflammation-associated gastrointestinal disorder. Gastric ulcers are benign mucosal lesions that penetrate deeply into the gut wall beyond the muscularis mucosae and form craters surrounded by acute and chronic inflammatory cell infiltrates. Many studies report that major risk factors for gastric ulcers include Helicobacter pylori infection, smoking, aspirin/non-steroidal anti-inflammatory drugs (NSAIDs) use, alcohol abuse and stress.

COX-2 is known as a proinflammatory enzyme and plays an important role in the regulation of several inflammatory and pain related conditions. COX-2 over-expression has been associated with neurotoxiticy in several conditions such as brain hypoxia/ischemia and seizures, as well as in inflammatory chronic diseases, including Creutzfeldt-Jakob disease, amyotrophic lateral sclerosis, multiple sclerosis, Parkinson's disease and Alzheimer's disease (Minghetti, L (2007); Minghetti (2004)). COX-2 also plays an important role in the regulation of the intestinal immune response. Traditionally considered as a proinflammatory enzyme, it has also long been recognized that COX-2 is upregulated in inflamed tissue in IBD patients.

JAKs and the family of intracellular transcription factors—signal transducers and activators of transcription (STATs)—combine to exert many cytokines' functions through activation of the ‘JAK-STAT’ pathway. Among the new drug targets, JAK inhibitors are a promising new class of drugs that have demonstrated efficacy with a favourable safety profile in clinical trials. Tofacitinib is the first JAK inhibitor approved for the treatment of ulcerative colitis.

TG2 is a calcium-dependent enzyme that catalyses polyamination of glutamine residues in proteins. TG2 is linked to IBD, and many other inflammatory diseases including celiac disease and sepsis. TG2 is also activated by oxidative stress caused by tissue injury, inflammation or hypoxia. TG2 has a role in triggering inflammation.

Conventional treatment for gastric ulcers includes pharmaceutical management with medicines such as omeprazole and ranitidine. Such medicines can have severe side effects such as myelosuppression and abnormal heart rhythm and are known to have high relapse rates.

There is therefore an interest in identifying other anti-inflammatory and analgesic agents for use in treating inflammation, pain and/or TG2, JAK, and/or COX-2 associated conditions. For example, anti-inflammatory and analgesic agents for use in treating gastrointestinal inflammation.

Honey is well-known for its anti-microbial activities. It is also suggested in the art that honey possesses anti-inflammatory activity, although the reason for this has not been well characterised. One patent publication, WO2015/030609, which is hereby incorporated by reference, explores the anti-inflammatory activity of a specific fraction of honey. This publication teaches that a low molecular weight fraction from honey has generalised anti-inflammatory effects and no immune-stimulatory effects. It does not discuss specific anti-inflammatory action.

It will be appreciated from the above that it would be useful to provide alternative methods of treating inflammatory conditions, including inflammatory conditions associated with the gastrointestinal tract.

It is an object of the invention to provide methods of treating inflammatory conditions, including inflammatory conditions associated with the gastrointestinal tract and/or to address on or more of the foregoing problems and/or at least to provide the public with a useful choice.

Further aspects and advantages of the products, compositions, methods and uses will become apparent from the ensuing description that is given by way of example only.

### SUMMARY OF THE INVENTION

Described herein are compositions comprising 3,6,7-trimethyllumazine, and methods of using the same for preventing, ameliorating or treating TG2, JAK, and/or COX-2 associated conditions, inflammation of the gastrointestinal tract, inflammatory conditions associated with gastrointestinal tract, and/or pain.

The inventors have identified that a pteridine from honey, 3,6,7-trimethyllumazine, has TG2, JAK, and/or COX-2 inhibitory activity. Being able to isolate the compound and characterise the anti-inflammatory and COX-2, TG2 and/or JAK inhibitory activity provides the ability to produce medicaments for various uses including the treatment, prevention and amelioration of conditions associated with TG2, JAK, and/or COX-2, including inflammatory conditions and pain. For example, inflammation and pain associated with the gastrointestinal tract.

In a first particular aspect, the invention provides a method of preventing, ameliorating or treating a COX-2 associated condition in a subject, comprising administering to a subject in need thereof a composition comprising 3,6,7-trimethyllumazine. In one embodiment, the COX-2 associated condition is an inflammatory condition. In one embodiment, the inflammatory condition is associated with inflammation of the gastrointestinal tract.

In one embodiment of the first aspect, the COX-2 associated condition is selected from the group consisting of; gastrointestinal inflammatory diseases, gastric ulcers, peptic ulcers, gastritis, inflammatory bowel disease (IBD), Crohn's disease, ulcerative colitis, irritable bowel syndrome (IBS), digestive disease, gastroesophageal reflux disease (GERD), heartburn, acid reflux, Helicobacter pylori infection, mouth ulcers, stomatitis, pharyngitis, gingivitis, esophageal ulcers, inflammatory and degenerative nervous system disorders, neuropsychiatric illnesses, schizophrenia, bipolar mood disorder, neurodegenerative disorders, traumatic brain injury, multiple sclerosis, Alzheimer's disease, nervous system disorders, Parkinson's disease, seizures, brain hypoxia/ischemia, Creutzfeldt-Jakob disease, amyotrophic lateral sclerosis, arthritis, rheumatoid arthritis, juvenile rheumatoid arthritis, ankylosing spondylitis, chronic inflammation, cardiovascular diseases, pain, cancer, colorectal cancer (CRC) and musculoskeletal diseases.

In one embodiment of the first aspect, the COX-2 associated condition is pain. In one embodiment, the pain is acute pain, chronic pain and/or dysmenorrhea.

In a second particular aspect, the invention provides a method of preventing, ameliorating or treating COX-2 associated inflammation in a subject comprising administering to a subject in need thereof a composition comprising 3,6,7-trimethyllumazine. In one embodiment, the inflammation is associated with the gastrointestinal tract of a subject.

In a third particular aspect, the invention provides a method of preventing, ameliorating or treating COX-2 associated pain in a subject comprising administering to a subject in need thereof a composition comprising 3,6,7-trimethyllumazine.

In another aspect, the invention provides a method of preventing, ameliorating or treating a TG2 associated condition in a subject, comprising administering to a subject in need thereof a composition comprising 3,6,7-trimethyllumazine. In one embodiment, the TG2 associated condition is selected from the group consisting of gastrointestinal inflammatory diseases, gastric ulcers (such as peptic ulcers), gastritis, TG2-associated inflammatory conditions, inflammatory bowel disease (IBD), Crohn's disease, ulcerative colitis, irritable bowel syndrome (IBS), digestive diseases, gastroesophageal reflux disease (GERD), heartburn, acid reflux, Helicobacter pylori infection, mouth ulcers, stomatitis, pharyngitis, gingivitis and/or esophageal ulcers, neuropsychiatric illnesses (such as schizophrenia and bipolar mood disorder), multiple sclerosis, neurodegenerative disorders (such as traumatic brain injury, multiple sclerosis, and Alzheimer's disease), cardiovascular diseases, cancer, arthritis, celiac disease, Huntington's disease, fibrosis and cancer. TG2 also plays a role in wound healing.

In another aspect, the invention provides a method of preventing, ameliorating or treating a JAK associated condition in a subject, comprising administering to a subject in need thereof a composition comprising 3,6,7-trimethyllumazine. In one embodiment, the JAK associated condition is selected from the group consisting of gastrointestinal inflammatory diseases, gastric ulcers (such as peptic ulcers), gastritis, JAK associated inflammatory conditions, inflammatory bowel disease (IBD), Crohn's disease, ulcerative colitis, irritable bowel syndrome (IBS), digestive diseases, gastroesophageal reflux disease (GERD), heartburn, acid reflux, Helicobacter pylori infection, mouth ulcers, stomatitis, pharyngitis, gingivitis and/or esophageal ulcers, neuropsychiatric illnesses (such as schizophrenia and bipolar mood disorder), multiple sclerosis, neurodegenerative disorders (such as traumatic brain injury, multiple sclerosis, and Alzheimer's disease), cardiovascular diseases, cancer, arthritis, chronic inflammation, auto-immune conditions, ulcerative colitis, alopecia areata, atopic dermatitis, diffuse scleroderma, Crohn's disease, vitiligo, hemophagocytic syndrome, non-infectious uveitis and cutaneous lupus erythematosus.

In one embodiment of the above aspects, the origin of the 3,6,7-trimethyllumazine is from Leptospermum. In one embodiment, the 3,6,7-trimethyllumazine is derived substantially from plants selected from the group consisting of: Leptospermum scoparium, Leptospermum polygalifolium, Leptospermum subtenue, and/or combinations thereof. In one embodiment, the 3,6,7-trimethyllumazine is from Leptospermum scoparium.

In one embodiment of the above aspects, the origin of the 3,6,7-trimethyllumazine is honey.

In one embodiment of the above aspects, the honey comprises honey of a floral origin substantially from the genus Leptospermum. In one embodiment, the honey comprises honey of a floral origin substantially from: Leptospermum scoparium, Leptospermum polygalifolium, Leptospermum subtenue, and/or combinations thereof. In one embodiment, the honey comprises honey of a floral origin substantially from Leptospermum scoparium (also referred to as Manuka).

In one embodiment of the above aspects, the honey is of a floral origin substantially from the genus Leptospermum. In one embodiment, the honey is of a floral origin substantially from: Leptospermum scoparium, Leptospermum polygalifolium, Leptospermum subtenue, and/or combinations thereof. In one embodiment, the honey is of a floral origin substantially from Leptospermum scoparium (also referred to as Manuka).

In one embodiment of the above aspects, the 3,6,7-trimethyllumazine is derived directly from a plant of the genus Leptospermum. In one embodiment, the 3,6,7-trimethyllumazine is derived directly from the flowers, nectar, roots, fruit, seeds, bark, oil, leaves, wood, stems or other plant material of a plant of the genus Leptospermum. In one embodiment, the 3,6,7-trimethyllumazine is substantially from plants selected from the group consisting of: Leptospermum scoparium, Leptospermum polygalifolium, Leptospermum subtenue, and/or combinations thereof.

In one embodiment of the above aspects, the composition comprising 3,6,7-trimethyllumazine comprises honey. In one embodiment, the composition comprising 3,6,7-trimethyllumazine consists of honey.

In one embodiment of the above aspects, the composition comprising 3,6,7-trimethyllumazine comprises a honey extract. In one embodiment of the above aspects, the composition comprising 3,6,7-trimethyllumazine comprises a honey extract, wherein the honey extract comprises a concentration of 3,6,7-trimethyllumazine that is higher than the concentration of 3,6,7-trimethyllumazine found naturally occurring in honey. In one embodiment, the composition consists of a honey extract, wherein the honey extract comprises a concentration of 3,6,7-trimethyllumazine that is higher than the concentration of 3,6,7-trimethyllumazine found naturally occurring in honey. In one embodiment, the honey extract comprises a concentration of 3,6,7-trimethyllumazine that is higher than the concentration of 3,6,7-trimethyllumazine found naturally occurring in the honey from which the extract was derived.

In one embodiment of the above aspects, the honey from which the extract is derived comprises honey of a floral origin substantially from the genus Leptospermum. In one embodiment, the honey from which the extract is derived comprises honey of a floral origin substantially from: Leptospermum scoparium, Leptospermum polygalifolium, Leptospermum subtenue, and/or combinations thereof. In one embodiment, the honey from which the extract is derived comprises honey of a floral origin substantially from Leptospermum scoparium. In one embodiment, the composition further comprises honey.

In one embodiment of the above aspects, the honey from which the extract is derived is of a floral origin substantially from the genus Leptospermum. In one embodiment, the honey from which the extract is derived is of a floral origin substantially from: Leptospermum scoparium, Leptospermum polygalifolium, Leptospermum subtenue, and/or combinations thereof. In one embodiment, the honey from which the extract is derived is of a floral origin substantially from Leptospermum scoparium. In one embodiment, the composition further comprises honey.

In one embodiment of the above aspects, the honey is raw honey, heat-treated honey or pasteurised honey.

In one embodiment of the above aspects, the composition comprises 3,6,7-trimethyllumazine isolated from honey. In one embodiment, the honey is of a floral origin substantially from the genus Leptospermum. In one embodiment, the honey is of a floral origin substantially from: Leptospermum scoparium, Leptospermum polygalifolium, Leptospermum subtenue, and/or combinations thereof. In one embodiment, the 3,6,7-trimethyllumazine is isolated by subjecting the honey to solid phase extraction, followed by normal-phase flash chromatography and preparative thin layer chromatography.

In one embodiment of the above aspects, the 3,6,7-trimethyllumazine is synthetic. In one embodiment, the composition further comprises honey.

In one embodiment of the above aspects, the composition comprises about 2.5 μg/mL to about 1000 μg/mL 3,6,7-trimethyllumazine. In one embodiment, the composition comprises 3,6,7-trimethyllumazine from about 2.5 μg/mL, about 5 μg/mL, about 10 μg/mL, about 20 μg/mL, about 40 μg/mL, about 50 μg/mL, about 60 μg/mL, about 70 μg/mL, about 80 μg/mL, about 90 μg/mL, about 100 μg/mL, 150 μg/mL, about 200 μg/mL, about 250 μg/mL, about 300 μg/mL, about 350 μg/mL, about 400 μg/mL, about 450 about 500 μg/mL, about 550 μg/mL, about 600 μg/mL, about 650 μg/mL, about 700 μg/mL, about 750 μg/mL, about 800 μg/mL, about 850 μg/mL, about 900 μg/mL or about 950 μg/mL, to about 1000 μg/mL, or the composition comprises about 2.5 to 5 μg/mL, about 5 to 10 μg/mL, about 10 to 20 μg/mL, about 20 to 40 μg/mL, about 40 to 50 μg/mL, about 50 to 60 μg/mL, about 60 to 70 μg/mL, about 70 to 80 μg/mL, about 80 to 90 μg/mL, about 90 to 100 μg/mL, about 100 to 150 μg/mL, 150 to 200 μg/mL, about 200 to 250 μg/mL, about 250 to 300 μg/mL, about 300 to 350 μg/mL, about 350 to 400 μg/mL, about 400 to 450 μg/mL, about 450 to 500 μg/mL, about 500 to 550 μg/mL, about 550 to 600 μg/mL, about 600 to 650 μg/mL, about 650 to 700 μg/mL, about 700 to 750 μg/mL, about 750 to 800 μg/mL, about 800 to 850 μg/mL, about 850 to 900 μg/mL, about 900 to 950 μg/mL or about 950 to 1000 μg/mL 3,6,7-trimethyllumazine.

In one embodiment of the above aspects, the composition comprises 3,6,7-trimethyllumazine about 5 mg/kg to about 3000 mg/kg. In one embodiment, the composition comprises 3,6,7-trimethyllumazine from about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 70 mg/kg, about 80 mg/kg, about 90 mg/kg, about 100 mg/kg, about 150 mg/kg, about 200 mg/kg, about 250 mg/kg, about 300 mg/kg, about 350 mg/kg, about 400 mg/kg, about 450 mg/kg, about 500 mg/kg, about 550 mg/kg, about 600 mg/kg, about 650 mg/kg, about 700 mg/kg, about 750 mg/kg, about 800 mg/kg, about 850 mg/kg, about 900 mg/kg, about 950 mg/kg, about 1000 mg/kg, about 1100 mg/kg, about 1200 mg/kg, about 1300 mg/kg, about 1400 mg/kg, about 1500 mg/kg, about 1600 mg/kg, about 1700 mg/kg, about 1800 mg/kg, about 1900 mg/kg, about 2000 mg/kg, about 2100 mg/kg, about 2200 mg/kg, about 2300 mg/kg, about 2400 mg/kg, about 2500 mg/kg, about 2600 mg/kg, about 2700 mg/kg, about 2800 mg/kg or about 2900 mg/kg to about 3000 mg/kg, or the composition comprises a concentration of 3,6,7-trimethyllumazine of about 5 to 10 mg/kg, about 10 to 15 mg/kg, about 15 to 20 mg/kg, about 20 to 25 mg/kg, about 25 to 30 mg/kg, about 30 to 35 mg/kg, about 35 to 40 mg/kg, about 40 to 45 mg/kg, about 45 to 50 mg/kg, about 50 to 55 mg/kg, about 55 to 60 mg/kg, about 60 to 70 mg/kg or about 70 to 80 mg/kg, about 90 to 100 mg/kg, about 100 to 150 mg/kg, about 150 to 200 mg/kg, about 200 mg/kg, about 250 to 300 mg/kg, about 300 to 350 mg/kg, about 350 to 400 mg/kg, about 400 to 450 mg/kg, about 450 to 500 mg/kg, about 500 to 550 mg/kg, about 550 to 600 mg/kg, about 600 to 650 mg/kg, about 650 to 700 mg/kg, about 700 to 750 mg/kg, about 750 to 800 mg/kg, about 800 to 850 mg/kg, about 850 to 900 mg/kg, about 900 to 950 mg/kg, about 950 to 1000 mg/kg, about 1000 to 1100 mg/kg, about 1100 to 1200 mg/kg, about 1200 to 1300 mg/kg, about 1300 to 1400 mg/kg, about 1400 to 1500 mg/kg, about 1500 to 1600 mg/kg, about 1600 to 1700 mg/kg, about 1700 to 1800 mg/kg, about 1800 to 1900 mg/kg, about 1900 to 2000 mg/kg, about 2000 to 2100 mg/kg, about 2100 to 2200 mg/kg, about 2200 to 2300 mg/kg, about 2300 to 2400 mg/kg, about 2400 to 2500 mg/kg, about 2500 to 2600 mg/kg, about 2600 to 2700 mg/kg, about 2700 to 2800 mg/kg, about 2800 to 2900 mg/kg or about 2900 to 3000 mg/kg.

In one embodiment of the above aspects, the composition comprises a therapeutically effective amount of 3,6,7-trimethyllumazine.

In one embodiment of the above aspects, the composition comprising 3,6,7-trimethyllumazine is formulated as a medicament, therapeutic product or health supplement. The composition comprising 3,6,7-trimethyllumazine may be formulated into a range of delivery systems, including but not limited to, liquid formulations, capsules, chewable tablets, tablets, suppositories, fast moving consumer goods, intravenous preparations, intramuscular preparations, subcutaneous preparations, solutions, food, beverages, dietary supplements, cosmetic formulations, gels, lotions, powders and sprays.

In one embodiment of the above aspects, the method comprises administering the composition comprising 3,6,7-trimethyllumazine one, two, three, four or five times daily.

In one embodiment of the above aspects, the method comprises administering the composition comprising 3,6,7-trimethyllumazine one, two, three, four, five, six or seven times weekly.

In one embodiment of the above aspects, the composition comprising 3,6,7-trimethyllumazine is administered as a single dose or as a divided dose. In one embodiment, the composition comprising 3,6,7-trimethyllumazine is administered as one, two, three or four separate doses.

In one embodiment of the above aspects, the method comprises administration of the composition comprising 3,6,7-trimethyllumazine at a dose of about 1 mg to about 3000 mg. In one particular embodiment, the method comprises administration of the composition comprising about 1 mg, 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg or 3000 mg 3,6,7-trimethyllumazine.

In one embodiment of the above aspects, the method comprises administering the composition at a dose of about 5 g to about 100 g of the composition comprising 3,6,7-trimethyllumazine.

In one embodiment of the above aspects, the composition comprising 3,6,7-trimethyllumazine has a standardised concentration of 3,6,7-trimethyllumazine obtained by:
a. selecting a first composition with a known concentration of 3,6,7-trimethyllumazine;
b. selecting at least one further composition with a known concentration of 3,6,7-trimethyllumazine; and
c. combining the first composition with the second composition to obtain a final composition with a standardised 3,6,7-trimethyllumazine concentration of about 5 mg/kg to about 3000 mg/kg.

In one embodiment of the above aspects, the composition comprising 3,6,7-trimethyllumazine has a standardised concentration of 3,6,7-trimethyllumazine obtained by:
a. selecting a first composition with a known concentration of 3,6,7-trimethyllumazine;
b. combining the selected first composition with one or more of:
   synthetic 3,6,7-trimethyllumazine;
   isolated 3,6,7-trimethyllumazine;
   a honey extract comprising 3,6,7-trimethyllumazine; and/or
   3,6,7-trimethyllumazine derived directly from a plant of the genus Leptospermum;
to form a composition with a standardised 3,6,7-trimethyllumazine concentration of about 5 mg/kg to about 3000 mg/kg.

In one embodiment of the above aspects, the composition comprises honey, a honey extract, isolated 3,6,7-trimethyllumazine and/or synthetic 3,6,7-trimethyllumazine.

In one embodiment of the above aspects, the 3,6,7-trimethyllumazine derived directly from a plant is derived directly from the flowers, nectar, roots, fruit, seeds, bark, oil, leaves, wood, stems or other plant material of a plant of the genus Leptospermum.

In one embodiment of the above aspects, the standardised 3,6,7-trimethyllumazine concentration is about 5 mg/kg to about 3000 mg/kg. In one embodiment, the standardised 3,6,7-trimethyllumazine concentration is: about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 70 mg/kg, about 80 mg/kg, about 90 mg/kg, about 100 mg/kg, about 150 mg/kg, about 200 mg/kg, about 250 mg/kg, about 300 mg/kg, about 350 mg/kg, about 400 mg/kg, about 450 mg/kg, about 500 mg/kg, about 550 mg/kg, about 600 mg/kg, about 650 mg/kg, about 700 mg/kg, about 750 mg/kg, about 800 mg/kg, about 850 mg/kg, about 900 mg/kg, about 950 mg/kg, about 1000 mg/kg, about 1100 mg/kg, about 1200 mg/kg, about 1300 mg/kg, about 1400 mg/kg, about 1500 mg/kg, about 1600 mg/kg, about 1700 mg/kg, about 1800 mg/kg, about 1900 mg/kg, about 2000 mg/kg, about 2100 mg/kg, about 2200 mg/kg, about 2300 mg/kg, about 2400 mg/kg, about 2500 mg/kg, about 2600 mg/kg, about 2700 mg/kg, about 2800 mg/kg or about 2900 mg/kg to about 3000 mg/kg of 3,6,7-trimethyllumazine.

In one embodiment of the above aspects, the concentration of the 3,6,7-trimethyllumazine is determined by chromatography, analytical measurements, spectrophotometry and/or any other method known to a person skilled in the art.

In one embodiment, the concentration of 3,6,7-trimethyllumazine is determined by reverse-phase HPLC.

In a fourth particular aspect the invention provides a method of making a composition with anti-inflammatory, analgesic and/or TG2, JAK, and/or COX-2 inhibitory activity comprising:
a. testing a first composition comprising honey for 3,6,7-trimethyllumazine concentration;
b. testing at least one further composition comprising honey for 3,6,7-trimethyllumazine concentration;
c. selecting a composition comprising honey with a 3,6,7-trimethyllumazine concentration greater than about 5 mg/kg 3,6,7-trimethyllumazine;
d. selecting at least one further composition comprising honey with a 3,6,7-trimethyllumazine concentration greater than about 5 mg/kg;
e. combining the selected composition comprising honey to form a honey composition with a 3,6,7-trimethyllumazine concentration of about 5 mg/kg to about 80 mg/kg.

In one embodiment of the fourth aspect, a composition comprising honey is selected if it has a concentration of 3,6,7-trimethyllumazine greater than about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 70 mg/kg or about 80 mg/kg.

In one embodiment of the fourth aspect, the composition comprises, consists essentially of, or consists of honey.

In one embodiment, the 3,6,7-trimethyllumazine concentration is determined by chromatography, analytical measurements, spectrophotometry and/or any other method known to a person skilled in the art. In one embodiment, the concentration of 3,6,7-trimethyllumazine is determined by reverse-phase HPLC.

In one embodiment of the fourth aspect, the composition with anti-inflammatory, analgesic and/or TG2, JAK and/or COX-2 inhibitory activity is suitable for use in the method of any one of the first, second or third aspects.

In a fifth particular aspect the invention provides a method of identifying a composition as having anti-inflammatory, analgesic and/or TG2, JAK, and/or COX-2 inhibitory activity comprising:
a. testing a composition for 3,6,7-trimethyllumazine concentration; and
   i. identifying the composition as having anti-inflammatory, analgesic, and/or TG2, JAK, and/or COX-2 inhibitory activity if it contains a 3,6,7-trimethyllumazine concentration greater than about 5 mg/kg; or
   ii. identifying the composition as not having anti-inflammatory, analgesic, and/or TG2, JAK, and/or COX-2 inhibitory activity if it contains a 3,6,7-trimethyllumazine concentration lower than about 5 mg/kg.

In one embodiment, the composition comprises honey or a honey extract.

In one embodiment of the fifth aspect, the composition comprising honey is determined as having anti-inflammatory activity if it contains greater than about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 70 mg/kg or about 80 mg/kg.

In one embodiment of the fifth aspect, the composition comprises, consists essentially of, or consists of honey or a honey extract.

In one embodiment of the fifth aspect, the composition with anti-inflammatory activity is suitable for use in the methods of any one of the first to third aspects.

In a sixth particular aspect, the invention provides a method of identifying a composition with anti-inflammatory, analgesic and/or TG2, JAK, and/or COX-2 inhibitory activity suitable for use in a method of any of the first to third aspects, comprising:
a. testing a composition for 3,6,7-trimethyllumazine concentration; and
   i. identifying the composition as suitable for use in any of the first to third aspects if it contains a 3,6,7-trimethyllumazine concentration about 5 to about 80 mg/kg 3,6,7-trimethyllumazine; or
   ii. identifying the composition as not suitable for use in any of the first to third aspects if it contains a 3,6,7-trimethyllumazine concentration lower than about 5 mg/kg 3,6,7-trimethyllumazine.

In one embodiment of the sixth aspect, the composition is identified as suitable for use in a method of any one of aspects one to three if it contains a 3,6,7-trimethyllumazine concentration greater than about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 70 mg/kg or about 80 mg/kg.

In one embodiment, the composition comprises, consists essentially of, or consists of honey or a honey extract.

In one embodiment, the 3,6,7-trimethyllumazine concentration is determined by chromatography, analytical measurements, spectrophotometry and/or any other method known to a person skilled in the art. In one embodiment, the concentration of 3,6,7-trimethyllumazine is determined by reverse-phase HPLC system.

In a seventh particular aspect, the invention provides a composition comprising 3,6,7-trimethyllumazine suitable for use in the method of any one of the first, second or third aspects.

In one embodiment of the seventh aspect, the origin of the 3,6,7-trimethyllumazine is from Leptospermum. In one embodiment, the 3,6,7-trimethyllumazine is substantially from plants selected from the group consisting of: Leptospermum scoparium, Leptospermum polygalifolium, Leptospermum subtenue, and/or combinations thereof. In one embodiment, the 3,6,7-trimethyllumazine is from Leptospermum scoparium.

In one embodiment of the seventh aspect, the origin of the 3,6,7-trimethyllumazine is honey. In one embodiment, the honey is of a floral origin substantially from the genus Leptospermum. In one embodiment, the honey is of a floral origin substantially from: Leptospermum scoparium, Leptospermum polygalifolium, Leptospermum subtenue, and/or combinations thereof. In one embodiment, the honey is of a floral origin substantially from Leptospermum scoparium (Manuka).

In one embodiment of the seventh aspect, the 3,6,7-trimethyllumazine is derived directly from a plant of the genus Leptospermum. In one embodiment, the 3,6,7-trimethyllumazine is derived directly from the nectar, roots, fruit, seeds, bark, oil, leaves, wood, stems or other plant material of a plant of the genus Leptospermum. In one embodiment, the 3,6,7-trimethyllumazine is substantially from plants selected from the group consisting of: Leptospermum scoparium, Leptospermum polygalifolium, Leptospermum subtenue, and/or combinations thereof.

In one embodiment of the seventh aspect, the composition comprising 3,6,7-trimethyllumazine comprises honey. In one embodiment, the composition comprising 3,6,7-trimethyllumazine consists essentially of honey. In one embodiment, the composition comprising 3,6,7-trimethyllumazine consists of honey.

In one embodiment of the above aspects, the composition comprising 3,6,7-trimethyllumazine comprises a honey extract. In one embodiment, the composition comprising 3,6,7-trimethyllumazine comprises a honey extract, wherein the honey extract comprises a concentration of 3,6,7-trimethyllumazine that is higher than the concentration of 3,6,7-trimethyllumazine found naturally occurring in honey. In one embodiment, the composition comprising 3,6,7-trimethyllumazine consists essentially of a honey extract, wherein the honey extract comprises a concentration of 3,6,7-trimethyllumazine that is higher than the concentration of 3,6,7-trimethyllumazine found naturally occurring in honey. In one embodiment, the composition comprising 3,6,7-trimethyllumazine consists of a honey extract, wherein the honey extract comprises a concentration of 3,6,7-trimethyllumazine that is higher than the concentration of 3,6,7-trimethyllumazine found naturally occurring in honey. In one embodiment, the honey extract comprises a concentration of 3,6,7-trimethyllumazine that is higher than the concentration of 3,6,7-trimethyllumazine found naturally occurring in the honey from which the extract was derived.

In one embodiment, the honey from which the extract is derived is of a floral origin substantially from the genus Leptospermum. In one embodiment, the honey from which the extract is derived is of a floral origin substantially from: Leptospermum scoparium, Leptospermum polygalifolium, Leptospermum subtenue, and/or combinations thereof. In one embodiment, the composition further comprises honey.

In one embodiment, the honey is raw honey, heat-treated honey or pasteurised honey.

In one embodiment, the composition comprises 3,6,7-trimethyllumazine isolated from honey. In one embodiment, the honey is of a floral origin substantially from the Leptospermum. In one embodiment, the honey is of a floral origin substantially from: Leptospermum scoparium, Leptospermum polygalifolium, Leptospermum subtenue, and/or combinations thereof. In one embodiment, the 3,6,7-trimethyllumazine is isolated by subjecting the honey to solid phase extraction, followed by normal-phase flash chromatography and preparative thin layer chromatography.

In one embodiment, the composition comprises synthetic 3,6,7-trimethyllumazine. In one embodiment, the composition further comprises honey.

In one embodiment, the composition comprises about 2.5 μg/mL to about 1000 μg/mL 3,6,7-trimethyllumazine. In one embodiment, the composition comprises from about 2.5 μg/mL, about 5 μg/mL, about 10 μg/mL, about 20 μg/mL, about 40 μg/mL, about 50 μg/mL, about 60 μg/mL, about 70 μg/mL, about 80 μg/mL, about 90 μg/mL, about 100 μg/mL, 150 μg/mL, about 200 μg/mL, about 250 μg/mL, about 300 μg/mL, about 350 μg/mL, about 400 μg/mL, about 450 about 500 μg/mL, about 550 μg/mL, about 600 μg/mL, about 650 μg/mL, about 700 μg/mL, about 750 μg/mL, about 800 μg/mL, about 850 μg/mL, about 900 μg/mL or about 950 μg/mL or to about 1000 μg/mL 3,6,7-trimethyllumazine, or the composition comprises about 2.5 to 5 μg/mL, about 5 to 10 μg/mL, about 10 to 20 μg/mL, about 20 to 40 μg/mL, about 40 to 50 μg/mL, about 50 to 60 μg/mL, about 60 to 70 μg/mL, about 70 to 80 μg/mL, about 80 to 90 μg/mL, about 90 to 100 μg/mL, about 100 to 150 μg/mL, 150 to 200 μg/mL, about 200 to 250 μg/mL, about 250 to 300 μg/mL, about 300 to 350 μg/mL, about 350 to 400 μg/mL, about 400 to 450 μg/mL, about 450 to 500 μg/mL, about 500 to 550 μg/mL, about 550 to 600 μg/mL, about 600 to 650 μg/mL, about 650 to 700 μg/mL, about 700 to 750 μg/mL, about 750 to 800 μg/mL, about 800 to 850 μg/mL, about 850 to 900 μg/mL, about 900 to 950 μg/mL or about 950 to 1000 μg/mL 3,6,7-trimethyllumazine.

In one embodiment, the composition comprises 3,6,7-trimethyllumazine about 5 mg/kg to about 3000 mg/kg 3,6,7-trimethyllumazine. In one embodiment, the composition comprises 3,6,7-trimethyllumazine from about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 70 mg/kg, about 80 mg/kg, about 90 mg/kg, about 100 mg/kg, about 150 mg/kg, about 200 mg/kg, about 250 mg/kg, about 300 mg/kg, about 350 mg/kg, about 400 mg/kg, about 450 mg/kg, about 500 mg/kg, about 550 mg/kg, about 600 mg/kg, about 650 mg/kg, about 700 mg/kg, about 750 mg/kg, about 800 mg/kg, about 850 mg/kg, about 900 mg/kg, about 950 mg/kg, about 1000 mg/kg, about 1100 mg/kg, about 1200 mg/kg, about 1300 mg/kg, about 1400 mg/kg, about 1500 mg/kg, about 1600 mg/kg, about 1700 mg/kg, about 1800 mg/kg, about 1900 mg/kg, about 2000 mg/kg, about 2100 mg/kg, about 2200 mg/kg, about 2300 mg/kg, about 2400 mg/kg, about 2500 mg/kg, about 2600 mg/kg, about 2700 mg/kg, about 2800 mg/kg or about 2900 mg/kg to about 3000 mg/kg or the composition comprises a concentration of 3,6,7-trimethyllumazine of 5 to 10 mg/kg, or about 10 to 15 mg/kg, or about 15 to 20 mg/kg, or about 20 to 25 mg/kg, or about 25 to 30 mg/kg, or about 30 to 35 mg/kg, or about 35 to 40 mg/kg, or about 40 to 45 mg/kg, or about 45 to 50 mg/kg, or about 50 to 55 mg/kg, or about 55 to 60 mg/kg, or about 60 to 70 mg/kg or about 70 to 80 mg/kg, about 90 to 100 mg/kg, about 100 to 150 mg/kg, about 150 to 200 mg/kg, about 200 mg/kg, about 250 to 300 mg/kg, about 300 to 350 mg/kg, about 350 to 400 mg/kg, about 400 to 450 mg/kg, about 450 to 500 mg/kg, about 500 to 550 mg/kg, about 550 to 600 mg/kg, about 600 to 650 mg/kg, about 650 to 700 mg/kg, about 700 to 750 mg/kg, about 750 to 800 mg/kg, about 800 to 850 mg/kg, about 850 to 900 mg/kg, about 900 to 950 mg/kg, about 950 to 1000 mg/kg, about 1000 to 1100 mg/kg, about 1100 to 1200 mg/kg, about 1200 to 1300 mg/kg, about 1300 to 1400 mg/kg, about 1400 to 1500 mg/kg, about 1500 to 1600 mg/kg, about 1600 to 1700 mg/kg, about 1700 to 1800 mg/kg, about 1800 to 1900 mg/kg, about 1900 to 2000 mg/kg, about 2000 to 2100 mg/kg, about 2100 to 2200 mg/kg, about 2200 to 2300 mg/kg, about 2300 to 2400 mg/kg, about 2400 to 2500 mg/kg, about 2500 to 2600 mg/kg, about 2600 to 2700 mg/kg, about 2700 to 2800 mg/kg, about 2800 to 2900 mg/kg or about 2900 to 3000 mg/kg.

In one embodiment, the composition comprises a therapeutically effective amount of 3,6,7-trimethyllumazine.

In one embodiment, the composition comprising 3,6,7-trimethyllumazine is formulated as a medicament, therapeutic product or health supplement. The composition comprising 3,6,7-trimethyllumazine may be formulated into a range of delivery systems, including but not limited to, liquid formulations, fast moving consumer goods, capsules, chewable tablets, tablets, suppositories, intravenous preparations, intramuscular preparations, subcutaneous preparations, solutions, food, beverages, dietary supplements, cosmetic formulations, gels, lotions, powders or sprays.

In an eighth particular aspect, the present invention provides a use of a composition comprising 3,6,7-trimethyllumazine in the manufacture of a medicament for preventing, ameliorating or treating a COX-2 associated condition.

In one embodiment of the eighth aspect, the COX-2 associated condition is selected from the group consisting of; gastrointestinal inflammatory diseases, gastric ulcers, peptic ulcers, gastritis, inflammatory bowel disease (IBD), Crohn's disease, ulcerative colitis, irritable bowel syndrome (IBS), digestive disease, gastroesophageal reflux disease (GERD), heartburn, acid reflux, Helicobacter pylori infection, mouth ulcers, stomatitis, pharyngitis, gingivitis, esophageal ulcers, inflammatory and degenerative nervous system disorders, neuropsychiatric illnesses, schizophrenia, bipolar mood disorder, neurodegenerative disorders, traumatic brain injury, multiple sclerosis, Alzheimer's disease, nervous system disorders, Parkinson's disease, seizures, brain hypoxia/ischemia, Creutzfeldt-Jakob disease, amyotrophic lateral sclerosis, arthritis, rheumatoid arthritis, juvenile rheumatoid arthritis, ankylosing spondylitis, chronic inflammation, cardiovascular diseases, cancer, pain, colorectal cancer (CRC) and musculoskeletal diseases.

In one embodiment of the eighth aspect, the COX-2 associated condition is pain. In one embodiment, the pain is acute pain, chronic pain and dysmenorrhea.

In a ninth particular aspect, the invention provides a use of a composition comprising 3,6,7-trimethyllumazine in the manufacture of a medicament for preventing, ameliorating or treating COX-2 associated inflammation. In one embodiment, the inflammation is associated with the gastrointestinal tract.

In a tenth particular aspect, the invention provides a use of a composition comprising 3,6,7-trimethyllumazine in the manufacture of a medicament for preventing, ameliorating or treating COX-2 associated pain. In one embodiment, the pain is acute pain, chronic pain and/or dysmenorrhea.

In an eleventh particular aspect, the invention provides a use of a composition comprising 3,6,7-trimethyllumazine in the manufacture of a medicament for preventing, ameliorating or treating an TG2 and/or JAK associated condition.

In one embodiment of the eleventh aspect, the TG2 associated condition is selected from the group consisting of gastrointestinal inflammatory diseases, gastric ulcers (such as peptic ulcers), gastritis, TG2 associated inflammatory conditions, inflammatory bowel disease (IBD), Crohn's disease, ulcerative colitis, irritable bowel syndrome (IBS), digestive diseases, gastroesophageal reflux disease (GERD), heartburn, acid reflux, Helicobacter pylori infection, mouth ulcers, stomatitis, pharyngitis, gingivitis and/or esophageal ulcers, neuropsychiatric illnesses (such as schizophrenia and bipolar mood disorder), multiple sclerosis, neurodegenerative disorders (such as traumatic brain injury, multiple sclerosis, and Alzheimer's disease), cardiovascular diseases, cancer, arthritis, celiac disease, Huntington's disease, fibrosis, cancer and a wound.

In one embodiment of the eleventh aspect, the JAK associated condition is selected from the group consisting of gastrointestinal inflammatory diseases, gastric ulcers (such as peptic ulcers), gastritis, JAK associated inflammatory conditions, inflammatory bowel disease (IBD), Crohn's disease, ulcerative colitis, irritable bowel syndrome (IBS), digestive diseases, gastroesophageal reflux disease (GERD), heartburn, acid reflux, Helicobacter pylori infection, mouth ulcers, stomatitis, pharyngitis, gingivitis and/or esophageal ulcers, neuropsychiatric illnesses (such as schizophrenia and bipolar mood disorder), multiple sclerosis, neurodegenerative disorders (such as traumatic brain injury, multiple sclerosis, and Alzheimer's disease), cardiovascular diseases, cancer, arthritis, chronic inflammation, auto-immune conditions, ulcerative colitis, alopecia areata, atopic dermatitis, diffuse scleroderma, Crohn's disease, vitiligo, hemophagocytic syndrome, non-infectious uveitis and cutaneous lupus erythematosus.

In a twelfth particular aspect, there is provided a method, use or composition of any one of the above aspects, wherein the composition further comprises a COX-2 inhibitor.

In a thirteenth particular aspect, there is provided a method or use of any one of the above aspects, further comprising co-administration of a COX-2 inhibitor. Advantages of the above methods and uses may be varied. In some embodiments, the source of 3,6,7-trimethyllumazine is naturally occurring and able to be manufactured on a sustainable basis. 3,6,7-trimethyllumazine is not anticipated to have side effects and it may be formulated in a wide variety of ways for various methods of administration.

This invention may also be said broadly to consist in the parts, elements and features referred to or indicated in the specification of the application, individually or collectively, and any or all combinations of any two or more of said parts, elements and features, and where specific integers are mentioned herein which have known equivalents in the art to which this invention relates, such known equivalents are deemed to be incorporated herein as if individually set forth.

Further aspects of the invention, which should be considered in all its novel aspects, will become apparent to those skilled in the art upon reading of the following description which provides at least one example of a practical application of the invention.

### BRIEF DESCRIPTION OF THE DRAWINGS

Embodiments of the invention will now be described, by way of example only, with reference to the accompanying drawings in which:

FIG. 1 is a graph illustrating the fluorescence intensity generated by MMP-9 activity over the course of 10 min.

FIG. 2 is a graph illustrating the percentage inhibition of MMP-9 activity from 3,6,7-trimethyllumazine ranging from 2.5-40 μg/ml.

FIG. 3 is a graph illustrating the correlation between 3,6,7-trimethyllumazine concentration and MMP-9 inhibition.

FIG. 4 is a graph illustrating MMP-9 activity measured by absorbance at 412 nm over 120 min.

FIG. 5 is a graph illustrating the percentage inhibition by 3,6,7-trimethyllumazine on the activity of MMP-9.

FIG. 6 is a graph illustrating that there is no significant interaction between 3,6,7-trimethyllumazine and the chromogenic substrate (A) or the reaction product (B) over the course of 20 min.

FIG. 7 shows a typical gelatin gel zymography showing gels incubated in normal developing buffer (column 3-5), 3,6,7-trimethyllumazine supplemented buffer (column 6-8), and NNGH (column 9-11).

FIG. 8 is a graph illustrating the percentage inhibition from 3,6,7-trimethyllumazine using gelatin zymography (n=5).

FIG. 9 illustrates 3,6,7-trimethyllumazine was docked into the S′1 substrate binding pocket of MMP-9.

FIG. 10 illustrates the amount of 3,6,7-trimethyllumazine (ng/mL) during the gastric digestion of four Manuka honey samples (A, B, C, D) as a function of digestion time.

FIG. 11 illustrates the amount of 3,6,7-trimethyllumazine (ng/mL) during the intestinal digestion of four Manuka honey samples (A, B, C, D) as a function of digestion time.

FIG. 12 illustrates the amount of 3,6,7-trimethyllumazine (ng/mL) during the gastric digestion of four 50% Manuka honey samples (A, B, C, D) as a function of digestion time.

FIG. 13 illustrates the amount of 3,6,7-trimethyllumazine (ng/mL) during the intestinal digestion of four 50% Manuka honey samples (A, B, C, D) as a function of digestion time.

FIG. 14 illustrates the amount of 3,6,7-trimethyllumazine (ng/mL) during the gastric digestion of pure 3,6,7-trimethyllumazine as a function of digestion time.

FIG. 15 illustrates the amount of 3,6,7-trimethyllumazine (ng/mL) during the intestinal digestion of pure 3,6,7-trimethyllumazine as a function of digestion time.

FIG. 16 illustrates the effect of 3,6,7-trimethyllumazine (2.5-40 μg/mL) on cell viability.

FIG. 17 illustrates the effect of 3,6,7-trimethyllumazine on lipopolysaccharide (055:B5, 1 μg/mL) induced matrix metallopeptidase 9 (MMP-9) secretion in differentiated THP-1 cells (n=2 replicates) (based on the raw values).

FIG. 18 illustrates the effect of 3,6,7-trimethyllumazine on lipopolysaccharide (055:B5, 1 μg/mL) induced matrix metallopeptidase 9 (MMP-9) secretion in differentiated THP-1 cells (n=2 replicates) (based on the absolute values).

FIG. 19 illustrates crystal structure of human JAK1 (PDB ID: 6N7A).

FIG. 20 illustrates docked pose of KEV (purple and labelled as A) compared to the original pose (green and labelled as B).

FIG. 21 illustrates GoldScore and ChemScore distribution of reported poses for known actives (green and labelled as A), inactives (red and labelled as B) and 3,6,7-trimethyllumazine (yellow and labelled as C).

FIG. 22 illustrates highest ranking docked pose of 3,6,7-trimethyllumazine to 6N7A.

FIG. 23 illustrates crystal structure of human transglutaminase 2 (PDB ID: 1KV3).

FIG. 24 illustrates docked pose of GDP (purple and labelled as A) compared to the original pose (B).

FIG. 25 illustrates GoldScore and ChemScore distribution of reported poses for known actives (green and labelled as A), inactives (red and labelled as B) and 3,6,7-trimethyllumazine (yellow and labelled as C).

FIG. 26 illustrates highest ranking docked pose of 3,6,7-trimethyllumazine to 1KV3.

FIG. 27 is a graph illustrating percent cell viability as assessed by the WST-1 assay for THP-1 cells after treatment with Dexamethasone (Dex), Indomethacin (Indo) or by 3,6,7-Trimethyllumazine (12.5, 25, 50, & 100 μg/mL) and co-stimulation with LPS.

FIG. 28 illustrates protein expression of COX-2 in monocytes following LPS exposure and co-treatment with LPS in combination with dexamethasone, indomethacin or 3,6,7-trimethyllumazine. FIG. 28A provides representative Western Blots for COX-2 protein expression and FIG. 18B is a graph illustrating the relative protein expression of COX-2 in THP-1 cells exposed to the different interventions.

### DETAILED DESCRIPTION OF PREFERRED EMBODIMENTS

Described herein are compositions comprising 3,6,7-trimethyllumazine methods, and uses of the same for the preventing, ameliorating or treating inflammation, pain and/or inflammatory conditions. In particular, inflammation, pain or inflammatory TG2, JAK, and/or COX-2 associated conditions.

### Definitions

For the purposes of this specification, the term “comprising” as used in this specification means “consisting in whole or at least in part of”. When interpreting statements in this specification which include that term, the features, prefaced by that term in each statement, all need to be present, but other features can also be present. Related terms such as “comprise”, comprises and “comprised” are to be interpreted in the same manner.

The term “about” or “approximately” and grammatical variations thereof mean a quantity, level, degree, value, number, frequency, percentage, dimension, size, amount, weight or length that varies by as much as 30, 25, 20, 15, 10, 9, 8, 7, 6, 5, 4, 3, 2, or 1%.

The term “medicament” or grammatical variations thereof refers to medical products. The medical products include, but are not limited to, liquid formulations, capsules, tablets, chewable tablets, gels, lotions, powders, fast moving consumer goods, suppositories, cosmetic formulations, spray preparations, food preparations, beverages, intravenous preparations, intramuscular preparations, subcutaneous preparations, and solutions.

The term “therapeutic products” or grammatical variations thereof refer to products which help to support, heal or restore health. The products include, but are not limited to, fast moving consumer goods, liquid formulations, capsules, tablets, chewable tablets, gels, lotions, powders suppositories, spray preparations, food preparations, beverages, cosmetic formulations, intravenous preparations, intramuscular preparations, subcutaneous preparations and solutions.

The term “inflammatory condition” means a condition or disorder associated with unwanted and/or abnormal inflammation.

The term “inflammation” means a body's reaction that produces redness, warmth, swelling and/or pain as the result of infection, irritation, injury, disease, condition or other cause. Inflammation can also be characterised at a cellular level. Cellular inflammation may be characterised by production of various inflammatory mediators such as cytokines, chemokines or reactive nitrogen and oxygen species.

The term “anti-inflammatory” or grammatical variations thereof refer to the prevention, mitigation, quenching, calming, suppression or reduction of inflammation associated cytokines, chemokines, reactive nitrogen and oxygen species, when compared to the duration, grade or situation, where no anti-inflammatory compound or compounds were added. It also refers to the inflammation being prevented, mitigated, quenched, calmed or suppressed to the extent that there is reduced redness, warmth, swelling and/or pain, the reduced amount being relative to the duration, grade or situation, where no anti-inflammatory compound or compounds were added.

The term “therapeutically effective” with reference to an amount or dosage of a composition or medicament refers to an amount of a composition that is sufficient to effectively prevent, ameliorate or eliminate inflammation, pain or one of the conditions described herein, in a subject. The term should not be seen as limiting.

It may refer to an amount of a dosage of a composition or medicament that optimises the effects, for example anti-inflammatory effects, on a subject depending on desired application.

The term “health supplement” means a product intended to be supplemented into the diet of a subject.

The term “treatment” is to be considered in its broadest context. The term does not necessarily imply that a subject is treated until total recovery. Accordingly, “treatment” includes reducing, alleviating or ameliorating the symptoms or severity of a particular condition or preventing or otherwise reducing the risk of developing a particular condition. It may also include maintaining or promoting a complete or partial state of remission of a condition.

The term “raw honey” means honey which has either undergone minimal heat (for example <50° C.) treatment or not undergone any heat processing.

The term “standardised concentration” means a concentration that has been determined to meet a pre-determined concentration range.

As used herein the term “and/or” means “and” or “or”, or both.

As used herein “(s)” following a noun means the plural and/or singular forms of the noun.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7).

A “subject” may be human or a non-human animal. Non-limiting examples of non-human animals are companion animals (e.g. cats and dogs), horses and livestock such as cattle, sheep and deer.

As noted above, the inventors have identified that 3,6,7-trimethyllumazine, for example 3,6,7-trimethyllumazine found in honey, has anti-inflammatory activity. In particular, the inventors surprisingly discovered that 3,6,7-trimethyllumazine has anti-inflammatory effects. In particular, the inventors discovered that 3,6,7-trimethyllumazine has TG2, JAK, and/or COX-2 inhibitory activity. Being able to characterise the activity and stability of 3,6,7-trimethyllumazine provides the ability to produce compositions for preventing, ameliorating or treating inflammation, including preventing, ameliorating or treating various TG2, JAK, and/or COX-2 associated conditions and inflammatory conditions, in particular, inflammatory conditions of the gastrointestinal tract.

Pteridines are a group of compounds based on a pyrimido[4,5-b]pyrazine ring structure. The bicyclic compounds are naturally produced by many living organisms and are often referred to as pterins. Pteridines and pteridine derivatives may also be synthetically produced. Many pteridine derivatives play essential metabolic roles as enzymatic cofactors, including the synthesis of nucleic acids, amino acids, neurotransmitters, nitrogen monoxides as well as purine and aromatic amino acids.

3,6,7-Trimethyllumazine is a pteridine derivative from Leptospermum honey. The isolation, structural elucidation and synthesis of 3,6,7-trimethyllumazine has previously been described in New Zealand patent application Number 722140 (NZ 722140) filed by the same applicant, herein incorporated by reference.

Inflammation is a multifactorial phenomenon implicated in a wide range of diseases. In a healthy gut, the intestinal mucosa is in a state of controlled response regulated by an intricate balance of pro-inflammatory cytokines (for example tumour necrosis factor, TNF-α, Interferon, IFN-γ, IL-1, IL-6) and anti-inflammatory cytokines (for example IL-4, IL-10). Defects in this can facilitate the complex interplay involved between genetic, microbial and environmental factors culminating in a sustained activation of the immune/non-immune responses, resulting in active inflammation and tissue destruction. Failure to resolve inflammation is implicated in the pathogenesis of gastrointestinal inflammatory related conditions such as gastric ulcers, inflammatory bowel disease (IBD), Crohn's disease and ulcerative colitis.

Conditions which are associated with TG2, JAK, and/or COX-2 include a range of different conditions such as gastrointestinal inflammatory diseases, gastric ulcers, peptic ulcers, gastritis, inflammatory bowel disease (IBD), Crohn's disease, ulcerative colitis, irritable bowel syndrome (IBS), digestive disease, gastroesophageal reflux disease (GERD), heartburn, acid reflux, Helicobacter pylori infection, mouth ulcers, stomatitis, pharyngitis, gingivitis, esophageal ulcers, inflammatory and degenerative nervous system disorders, neuropsychiatric illnesses, schizophrenia, bipolar mood disorder, neurodegenerative disorders, traumatic brain injury, multiple sclerosis, Alzheimer's disease, nervous system disorders, Parkinson's disease, seizures, brain hypoxia/ischemia, Creutzfeldt-Jakob disease, amyotrophic lateral sclerosis, arthritis, rheumatoid arthritis, juvenile rheumatoid arthritis, ankylosing spondylitis, chronic inflammation, cardiovascular diseases, cancer, pain, colorectal cancer (CRC), musculoskeletal diseases, wounds, auto-immune conditions, alopecia areata, atopic dermatitis, diffuse scleroderma, vitiligo, hemophagocytic syndrome, non-infectious uveitis, cutaneous lupus erythematosus, celiac disease, Huntington's disease and fibrosis.

### MMP

One of the main roles of MMPs in inflammation is regulating physical barriers. Inflammatory cell migration is facilitated by MMPs due to their ability to digest intercellular junctions. Several major components of endothelial adherent junctions have been identified as substrates of MMPs. The disassembly of these cellular components increases vascular permeability thus allowing the influx of inflammatory cells and plasma proteins.

MMP-9 (also known as Gelatinase B) is a proinflammatory enzyme which can proteolytically process a number of cytokines and chemokines into more active forms, such as pro-IL-1l and IL-8 (Schonbeck et al., 1998; Van den Steen, Proost, Wuyts, Van Damme, & Opdenakker, 2000). It is also reported that MMP-9 can regulate epithelial barrier permeability by degrading occludins in tight junctions to facilitate the influx of inflammatory cells and proteins (Caron et al., 2005; Reijerkerk et al., 2006) and it is highly involved in extracellular matrix (ECM) degradation which leads to mucosal damage and cellular remodelling (Swarnakar et al., 2007). MMP-9 is associated with a number of conditions including neuropsychiatric illnesses (such as schizophrenia and bipolar mood disorder), multiple sclerosis, neurodegenerative disorders (such as traumatic brain injury, multiple sclerosis, and Alzheimer's disease), cardiovascular diseases, cancer and arthritis (Rybakowski 2009, Fingleton (2007), Reinhard, 2015).

MMP-9 is also highly associated with the occurrence and severity of gastric ulcers. Numerous studies have reported elevated expression and activity of MMP-9 during the process of gastric ulceration (Pradeepkumar Singh, Kundu, Ganguly, Mishra, & Swarnakar, 2007; Swarnakar et al., 2005, 2007). It is also reported that ethanol-induced gastric ulcers are associated with the elevation of pro-MMP-9 activity in a dose-, time- and severity-dependent manner and that MMP-9 a risk factor for the reoccurrence of gastric ulcers (Li et al., 2013).

The expression and secretion of MMP-9 is very low in normal healthy tissues. During the formation of a gastric ulcer, the induction of oxidative stress intensifies the secretion of MMP-9 and leads to mucosal damage (Ganguly & Swarnakar, 2012; Li et al., 2013). MMP-9 is thus a known therapeutic target in preventing and healing gastric ulceration.

MMP-9-associated conditions are therefore conditions in which there is an increase in expression of MMP-9, and include inflammatory conditions in which there is an increase in expression or overexpression of MMP-9. Such conditions include, but are not limited to, gastric ulcers (such as peptic ulcers), gastritis, other MMP-associated inflammatory conditions, inflammatory bowel disease (IBD), Crohn's disease, ulcerative colitis, irritable bowel syndrome (IBS), digestive diseases, gastroesophageal reflux disease (GERD), heartburn, acid reflux, Helicobacter pylori infection, mouth ulcers, stomatitis, pharyngitis, gingivitis and/or esophageal ulcers. In one particular embodiment, the MMP-9 associated inflammatory condition is gastric ulcers or gastritis. MMP-9 associated conditions also include other conditions such as including neuropsychiatric illnesses (such as schizophrenia and bipolar mood disorder), multiple sclerosis, neurodegenerative disorders (such as traumatic brain injury, multiple sclerosis, and Alzheimer's disease), cardiovascular diseases, cancer and arthritis.

### COX-2

COX-2 is a proinflammatory enzyme and is known to play an important role in the regulation of a number of inflammatory and pain related conditions.

COX-2 and the role it plays in neuro-inflammatory and degenerative diseases has been extensively studied. COX-2 over-expression has been associated with neurotoxicity in a number of conditions such as brain hypoxia/ischemia and seizures, as well as in inflammatory chronic diseases, including Creutzfeldt-Jakob disease, amyotrophic lateral sclerosis, Multiple Sclerosis, Parkinson's Disease and Alzheimer's disease (Minghetti, L (2007); Minghetti (2004)).

COX-2 also plays an important role in the regulation of the intestinal immune response. Upon the recognition of foreign agents, e.g., bacterial products, in the intestinal lumen by the TLRs (such as TLR4), COX-2 expression is induced by transcription factors such as NF-κB. COX-2 activation may impact inflammatory processes via the inhibition of NF-κB and activation of peroxisome proliferator-activated receptor γ (PPAR-γ) and by the modification of the mucosal barrier function. Traditionally considered as a proinflammatory, it has also long been recognized that COX-2 is upregulated in inflamed tissue in IBD patients.

COX-2 is also associated with the progression and development of cancer. For example, in colorectal cancer (CRC) patients COX-2 expression is found in most CRC tissue and is associated with worse survival (Wang et al., (2010)).

As will be appreciated from the above, COX-2 related conditions are conditions associated with the increase in expression or overexpression of COX-2. Such conditions include gastrointestinal inflammatory diseases, gastric ulcers (such as peptic ulcers), gastritis, inflammatory bowel disease (IBD), Crohn's disease, ulcerative colitis, irritable bowel syndrome (IBS), digestive disease, gastroesophageal reflux disease (GERD), heartburn, acid reflux, Helicobacter pylori infection, mouth ulcers, stomatitis, pharyngitis, gingivitis, esophageal ulcers, inflammatory and degenerative nervous system disorders, neuropsychiatric illnesses (such as schizophrenia and bipolar mood disorder), neurodegenerative disorders (such as traumatic brain injury, multiple sclerosis and Alzheimer's disease), nervous system disorders such as Parkinson's disease and/or seizures, brain hypoxia/ischemia, Creutzfeldt-Jakob disease, amyotrophic lateral sclerosis, arthritis (such as rheumatoid arthritis, juvenile rheumatoid arthritis and ankylosing spondylitis), chronic inflammation, cardiovascular diseases, pain, cancer (such as colorectal cancer (CRC)) and musculoskeletal diseases.

As will be appreciated from the above, the COX-2 related condition may also be COX-2 associated pain. For example, acute pain (such as pain caused by a physical injury), chronic pain and/or dysmenorrhea (pain associated with menstruation). The pain may also be pain associated with any one of the above COX-2 related conditions.

### JAK

JAKs are a family of four intracellular tyrosine kinases: JAK1, JAK2, JAK3, and tyrosine kinase 2 (TYK2). JAKs and the family of seven intracellular transcription factors—signal transducers and activators of transcription (STATs)—combine to exert many cytokines' functions through activation of the ‘JAK-STAT’ pathway. Following a cytokine's binding to the extracellular domain of its receptor, JAKs bind to the intracellular domain and activate. This leads to a recruitment, phosphorylation, and activation of intracytoplasmatic STATs, which allows them to translocate into the nucleus, and then modulate the expression of various target genes involved in the inflammation. Under current therapeutic management, a significant number of patients with IBD fail in achieving a sustained remission. Among the new drug targets, JAK inhibitors are a promising new class of drugs that have demonstrated efficacy with a favourable safety profile in clinical trials. Tofacitinib is the first JAK inhibitor approved for the treatment of ulcerative colitis.

As will be appreciated from the above, JAK related conditions are conditions associated with the activation of the ‘JAK-STAT’ pathway. Such conditions include, gastrointestinal inflammatory diseases, gastric ulcers (such as peptic ulcers), gastritis, JAK-associated inflammatory conditions, inflammatory bowel disease (IBD), Crohn's disease, ulcerative colitis, irritable bowel syndrome (IBS), digestive diseases, gastroesophageal reflux disease (GERD), heartburn, acid reflux, Helicobacter pylori infection, mouth ulcers, stomatitis, pharyngitis, gingivitis and/or esophageal ulcers, neuropsychiatric illnesses (such as schizophrenia and bipolar mood disorder), neurodegenerative disorders (such as traumatic brain injury, multiple sclerosis, and Alzheimer's disease), cardiovascular diseases, cancer, arthritis, chronic inflammation, auto-immune conditions, ulcerative colitis, alopecia areata, atopic dermatitis, diffuse scleroderma, Crohn's disease, vitiligo, hemophagocytic syndrome, non-infectious uveitis and cutaneous lupus erythematosus.

### TG2

TG2 is a calcium dependent enzyme that catalyses polyamination of glutamine residues in proteins. TG2 is linked is linked to IBD, and many other inflammatory diseases including celiac disease and sepsis. NF-kB is activated by TG2 which then polymerizes and therefore inactivates its inhibitor, IkBα, by cross-linking its C-terminal glutamine cluster. Pre-clinical studies have shown that TG2 can also promote inflammation through the aggregation and functional sequestration of PPARγ, where the specific in vitro inhibition of TG2 is able to reinstate PPARγ and inflammatory cytokine levels. TG2 is also activated by oxidative stress caused by tissue injury, inflammation or hypoxia. Post activation, the protein covalently activates many proteins resulting in the modulation of cell adhesion molecules, cytokines and other mediators involved in the cell survival. TG2 has a role in triggering inflammation and so down regulating its activity would likely be useful in treatment in IBD. Studies have also previously reported significant high levels of transglutaminase2 antibodies in the serum of patients with IBD.

As will be appreciated from the above, TG2 related conditions are conditions associated with the increase in expression or overexpression of TG2. Such conditions include, gastrointestinal inflammatory diseases, gastric ulcers (such as peptic ulcers), gastritis, TG2-associated inflammatory conditions, inflammatory bowel disease (IBD), Crohn's disease, ulcerative colitis, irritable bowel syndrome (IBS), digestive diseases, gastroesophageal reflux disease (GERD), heartburn, acid reflux, Helicobacter pylori infection, mouth ulcers, stomatitis, pharyngitis, gingivitis and/or esophageal ulcers, neuropsychiatric illnesses (such as schizophrenia and bipolar mood disorder), neurodegenerative disorders (such as traumatic brain injury, multiple sclerosis, and Alzheimer's disease), cardiovascular diseases, cancer, arthritis, celiac disease, Huntington's disease, fibrosis and cancer. TG2 also plays a role in wound healing.

As will be appreciated from the above, COX-2 is a desirable target for preventing, ameliorating or treating inflammation, pain and/or preventing, ameliorating or treating conditions associated with inflammation and/or pain. In particular, for preventing, ameliorating or treating conditions associated with inflammation of the gastrointestinal tract. COX-2 is also a desirable target for treating other conditions which are associated with COX-2, such as gastrointestinal inflammatory diseases, gastric ulcers (such as peptic ulcers), gastritis, inflammatory bowel disease (IBD), Crohn's disease, ulcerative colitis, irritable bowel syndrome (IBS), digestive disease, gastroesophageal reflux disease (GERD), heartburn, acid reflux, Helicobacter pylori infection, mouth ulcers, stomatitis, pharyngitis, gingivitis, esophageal ulcers, inflammatory and degenerative nervous system disorders, neuropsychiatric illnesses (such as schizophrenia and bipolar mood disorder), neurodegenerative disorders (such as traumatic brain injury, multiple sclerosis and Alzheimer's disease), nervous system disorders such as Parkinson's disease and/or seizures, brain hypoxia/ischemia, Creutzfeldt-Jakob disease, amyotrophic lateral sclerosis, arthritis (such as rheumatoid arthritis, juvenile rheumatoid arthritis and ankylosing spondylitis), chronic inflammation, cardiovascular diseases, pain (such as, acute pain (such as pain caused by a physical injury), chronic pain and/or dysmenorrhea (pain associated with menstruation)), cancer (such as colorectal cancer (CRC)) and musculoskeletal diseases.

The inventors have found that 3,6,7-trimethyllumazine and compositions comprising the same have COX-2 inhibitory activity and are therefore useful in methods of preventing, ameliorating or treating COX-2 associated conditions, such as those related to inflammation and/or pain. The inventors found that surprisingly, 3,6,7-trimethyllumazine inhibits the expression of COX-2. The COX-2 inhibitory effects are significant, suggesting good efficacy and potentially a broad range of applications and uses, in particular in the prevention and/or treatment of inflammation and pain. For example, in the treatment of inflammatory conditions, such as gastrointestinal inflammatory conditions including gastritis and gastric ulcers.

In one embodiment, the COX-2 associated condition is an inflammatory condition. In one embodiment, the inflammatory condition is associated with inflammation of the gastrointestinal tract. In one embodiment, the COX-2 associated condition is selected from gastrointestinal inflammatory diseases, gastric ulcers (such as peptic ulcers), gastritis, inflammatory bowel disease (IBD), Crohn's disease, ulcerative colitis, irritable bowel syndrome (IBS), digestive disease, gastroesophageal reflux disease (GERD), heartburn, acid reflux, Helicobacter pylori infection, mouth ulcers, stomatitis, pharyngitis, gingivitis, esophageal ulcers, inflammatory and degenerative nervous system disorders, neuropsychiatric illnesses (such as schizophrenia and bipolar mood disorder), neurodegenerative disorders (such as traumatic brain injury, multiple sclerosis and Alzheimer's disease), nervous system disorders such as Parkinson's disease and/or seizures, brain hypoxia/ischemia, Creutzfeldt-Jakob disease, amyotrophic lateral sclerosis, arthritis (such as rheumatoid arthritis, juvenile rheumatoid arthritis and ankylosing spondylitis), chronic inflammation, cardiovascular diseases, pain (such as, acute pain (such as pain caused by a physical injury), chronic pain and/or dysmenorrhea (pain associated with menstruation)), cancer (such as colorectal cancer (CRC)) and musculoskeletal diseases.

The inventors have also surprisingly discovered that 3,6,7-trimethyllumazine binds to and JAK, and may therefore be used to treat conditions associated with and JAK. In one embodiment, the condition is an inflammatory condition.

In one embodiment, the TG2 associated condition is selected from the group consisting of gastrointestinal inflammatory diseases, gastric ulcers (such as peptic ulcers), gastritis, TG2-associated inflammatory conditions, inflammatory bowel disease (IBD), Crohn's disease, ulcerative colitis, irritable bowel syndrome (IBS), digestive diseases, gastroesophageal reflux disease (GERD), heartburn, acid reflux, Helicobacter pylori infection, mouth ulcers, stomatitis, pharyngitis, gingivitis and/or esophageal ulcers, neuropsychiatric illnesses (such as schizophrenia and bipolar mood disorder), multiple sclerosis, neurodegenerative disorders (such as traumatic brain injury, multiple sclerosis, and Alzheimer's disease), cardiovascular diseases, cancer, arthritis, celiac disease, Huntington's disease, fibrosis, cancer and a wound.

In one embodiment, the JAK associated condition is selected from the group consisting of gastrointestinal inflammatory diseases, gastric ulcers (such as peptic ulcers), gastritis, JAK-associated inflammatory conditions, inflammatory bowel disease (IBD), Crohn's disease, ulcerative colitis, irritable bowel syndrome (IBS), digestive diseases, gastroesophageal reflux disease (GERD), heartburn, acid reflux, Helicobacter pylori infection, mouth ulcers, stomatitis, pharyngitis, gingivitis and/or esophageal ulcers, neuropsychiatric illnesses (such as schizophrenia and bipolar mood disorder), multiple sclerosis, neurodegenerative disorders (such as traumatic brain injury, multiple sclerosis, and Alzheimer's disease), cardiovascular diseases, cancer, arthritis, chronic inflammation, auto-immune conditions, ulcerative colitis, alopecia areata, atopic dermatitis, diffuse scleroderma, Crohn's disease, vitiligo, hemophagocytic syndrome, non-infectious uveitis and cutaneous lupus erythematosus.

In another aspect, the invention provides a method of preventing, ameliorating or treating inflammation of the gastrointestinal tract in a subject comprising administering to a subject in need thereof a composition comprising 3,6,7-trimethyllumazine. In one embodiment the inflammation is COX-2 associated inflammation. In one embodiment, the inflammation is associated with the gastrointestinal tract of a subject.

In another aspect, the invention provides a method of preventing, ameliorating or treating COX-2 associated pain in a subject comprising administering to a subject in need thereof a composition comprising 3,6,7-trimethyllumazine. In one embodiment, the pain is acute pain, chronic pain and/or dysmenorrhea.

In another aspect, the invention provides a method of preventing, ameliorating or treating conditions associated with inflammation of the gastrointestinal tract.

In one aspect, the invention provides a method of preventing, ameliorating or treating inflammation in a subject comprising administering to a subject in need thereof a composition comprising 3,6,7-trimethyllumazine. In one embodiment, the inflammation is inflammation of the gastrointestinal tract.

In one embodiment, the invention provides a method of preventing, ameliorating or treating inflammation associated with a COX-2 associated condition, a TG2 associated condition and/or a JAK associated condition.

In one embodiment, the invention provides a method of preventing, ameliorating or treating conditions such as a COX-2 associated condition, a TG2 associated condition and/or a JAK associated condition.

In one embodiment, the COX-2 associated condition is selected from the group consisting of gastrointestinal inflammatory diseases, gastric ulcers (such as peptic ulcers), gastritis, inflammatory bowel disease (IBD), Crohn's disease, ulcerative colitis, irritable bowel syndrome (IBS), digestive disease, gastroesophageal reflux disease (GERD), heartburn, acid reflux, Helicobacter pylori infection, mouth ulcers, stomatitis, pharyngitis, gingivitis, esophageal ulcers, inflammatory and degenerative nervous system disorders, neuropsychiatric illnesses (such as schizophrenia and bipolar mood disorder), neurodegenerative disorders (such as traumatic brain injury, multiple sclerosis and Alzheimer's disease), nervous system disorders such as Parkinson's disease and/or seizures, brain hypoxia/ischemia, Creutzfeldt-Jakob disease, amyotrophic lateral sclerosis, arthritis (such as rheumatoid arthritis, juvenile rheumatoid arthritis and ankylosing spondylitis), chronic inflammation, cardiovascular diseases, pain (such as, acute pain (such as pain caused by a physical injury), chronic pain and dysmenorrhea (pain associated with menstruation)), cancer (such as colorectal cancer (CRC)) and musculoskeletal diseases.

In one embodiment, the TG2 associated condition is selected from the group consisting of gastrointestinal inflammatory diseases, gastric ulcers (such as peptic ulcers), gastritis, TG2 associated inflammatory conditions, inflammatory bowel disease (IBD), Crohn's disease, ulcerative colitis, irritable bowel syndrome (IBS), digestive diseases, gastroesophageal reflux disease (GERD), heartburn, acid reflux, Helicobacter pylori infection, mouth ulcers, stomatitis, pharyngitis, gingivitis and/or esophageal ulcers, neuropsychiatric illnesses (such as schizophrenia and bipolar mood disorder), multiple sclerosis, neurodegenerative disorders (such as traumatic brain injury, multiple sclerosis, and Alzheimer's disease), cardiovascular diseases, cancer, arthritis, celiac disease, Huntington's disease, fibrosis, cancer and a wound.

In one embodiment, the JAK associated condition is selected from the group consisting of gastrointestinal inflammatory diseases, gastric ulcers (such as peptic ulcers), gastritis, JAK associated inflammatory conditions, inflammatory bowel disease (IBD), Crohn's disease, ulcerative colitis, irritable bowel syndrome (IBS), digestive diseases, gastroesophageal reflux disease (GERD), heartburn, acid reflux, Helicobacter pylori infection, mouth ulcers, stomatitis, pharyngitis, gingivitis and/or esophageal ulcers, neuropsychiatric illnesses (such as schizophrenia and bipolar mood disorder), multiple sclerosis, neurodegenerative disorders (such as traumatic brain injury, multiple sclerosis, and Alzheimer's disease), cardiovascular diseases, cancer, arthritis, chronic inflammation, auto-immune conditions, ulcerative colitis, alopecia areata, atopic dermatitis, diffuse scleroderma, Crohn's disease, vitiligo, hemophagocytic syndrome, non-infectious uveitis and cutaneous lupus erythematosus.

As will be appreciated from the above, 3,6,7-trimethyllumazine, and compositions comprising the same, may be useful in a wide range of other uses, including for supporting or maintaining a subject's normal digestion, supporting or maintaining a subject's healthy digestion and supporting or maintaining a subject's general gut health and wellbeing.

In one embodiment of the invention, the origin of the 3,6,7-trimethyllumazine in the methods, uses and compositions disclosed herein is from Leptospermum. In one embodiment, the 3,6,7-trimethyllumazine is substantially from plants selected from the group consisting of: Leptospermum scoparium, Leptospermum polygalifolium, Leptospermum subtenue, and/or combinations thereof. In one embodiment, the 3,6,7-trimethyllumazine is from Leptospermum scoparium (Manuka).

In one embodiment of the invention, the origin of the 3,6,7-trimethyllumazine is honey. In one embodiment, the honey is of a floral origin substantially from the genus Leptospermum. In one embodiment, the honey is of a floral origin substantially from: Leptospermum scoparium, Leptospermum polygalifolium, Leptospermum subtenue, and/or combinations thereof.

In one embodiment, the honey is of a floral origin substantially from Leptospermum scoparium (also referred to as Manuka).

In one particular embodiment, the 3,6,7-trimethyllumazine is derived directly from a plant of the genus Leptospermum. In one embodiment of the invention, the 3,6,7-trimethyllumazine is derived directly from the nectar, roots, fruit, seeds, bark, oil, leaves, wood, stems or other plant material of a plant of the genus Leptospermum. In one embodiment of the invention, the 3,6,7-trimethyllumazine is derived directly from the nectar of a plant of the genus Leptospermum. In one aspect, the 3,6,7-trimethyllumazine is substantially from plants selected from the group consisting of: Leptospermum scoparium, Leptospermum polygalifolium, Leptospermum subtenue, and/or combinations thereof.

In one embodiment of the invention, the 3,6,7-trimethyllumazine is synthetic.

For example, the 3,6,7-trimethyllumazine may be synthesised as described in NZ 722140, and as shown below.

Referring to the below scheme, and following the work of Gala et al, (1997) N-methylation of 6-aminouracil (5) at position 3 was accomplished via silylation of the exocyclic amino and carbonyl groups upon treatment with hexamethyldisilazane (HDMS) in the presence of a catalytic amount of sulfuric acid (H₂SO₄). Ammonium sulfate could also be used as a catalyst. Methylation was then effected using iodomethane (Mel) in the presence of dimethylformamide (DMF) as an organic solvent in a 71% yield over two steps. Dimethylsulfate could also be used as a methylating agent. Subsequent desilylation during aqueous workup afforded 6-amino-3-methyluracil (6) in 78% yield.

Amino uracil (6) was then treated with sodium nitrite (NaNO₂) and acetic acid (AcOH) solution, followed by reduction with sodium dithionite (Na₂S₂O₄) in the aqueous solvent ammonia (NH₃) at 70° C. (Chaudhari et al., 2009) to give 5,6-diamino-3-methyluracil (7) in 31% yield over two steps. Alternative acids which could be used in the nitrosation first step include hydrochloric acid. An alternative to the first step reduction with sodium nitrite and acetic acid is catalytic hydrogenation using a catalyst such as palladium on carbon or platinum dioxide in an aqueous or organic solvent.

Condensation of diaminouracil (7) with 2,3-butanedione (8) in ethanol (EtOH) and acetic acid (AcOH) solution gave 3,6,7-trimethyllumazine (3) as a colourless solid. An alternative acid for use in the condensation step is hydrochloric acid. Spectroscopic data (UV-vis, IR, ¹H NMR and ¹³C NMR) of synthetic 3,6,7-trimethyllumazine was in excellent agreement with that of the isolated natural product. Furthermore, the ¹H NMR spectra of both the natural and synthetic products were identical. Thus the structure of 3,6,7-trimethyllumazine (3) was definitively established as 3,6,7-trimethyllumazine.

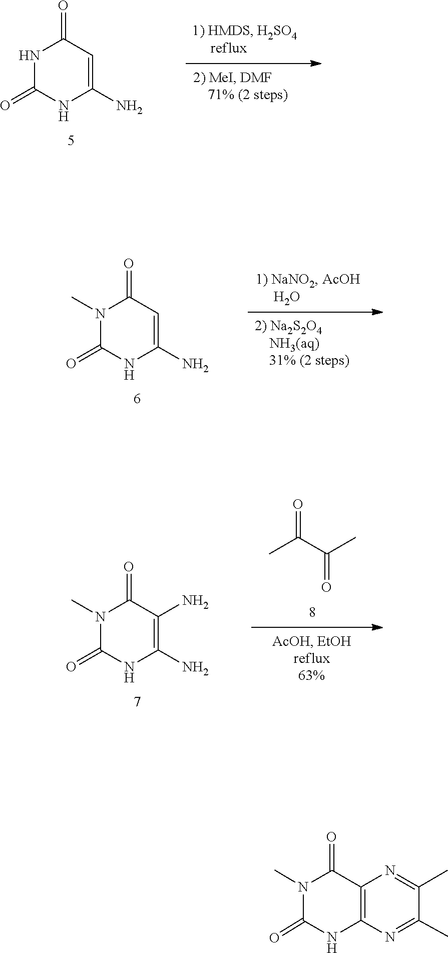

Alternative synthesis of compound (9)

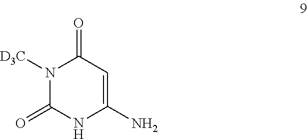 [SMILES: [2H]C([2H])([2H])n1c(=O)cc(N)[nH]c1=O]

is via methylation at N-3 of the

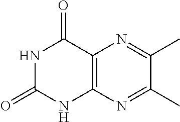

intermediate compound shown above or via transformation of the intermediate compound shown below into a transient isocyanate species, including but not limited to those generated by a Curtius, Hofmann, Lossen or Schmidt rearrangement.

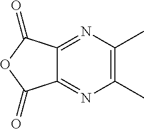

Referring to the above, N-deuteromethylation of 6-aminouracil (5) at position 3 was accomplished via silylation of the exocyclic amino and carbonyl groups upon treatment with hexamethyldisilazane (HDMS) in the presence of a catalytic amount of sulfuric acid (H₂SO₄). Methylation was then effected using iodomethane-d₃ (CD₃I) in the presence of dimethylformamide (DMF) as an organic solvent in a 71% yield over two steps. Subsequent desilylation during aqueous workup afforded 6-amino-3-(²H₃)methyluracil (9) in 78% yield.

Amino uracil (6) was then treated with sodium nitrite (NaNO₂) and acetic acid (AcOH) solution, followed by reduction with sodium dithionite (Na₂S₂O₄) in the aqueous solvent ammonia (NH₃) at 70° C. (Chaudhari et al., 2009) to give 5,6-diamino-3-(²H₃)methyluracil (10) in 31% yield over two steps. Alternative acids which could be used in the nitrosation first step include hydrochloric acid. An alternative to the first step reduction with sodium nitrite and acetic acid is catalytic hydrogenation using a catalyst such as palladium on carbon or platinum dioxide in an aqueous or organic solvent.

Condensation of diamino uracil (10) with 2,3-butanedione (8) in ethanol (EtOH) and acetic acid (AcOH) solution gave 3,6,7-(3-²H₃)trimethyllumazine (11) as a colourless solid.

### Materials and Methods

All reactions were carried out in flame- or oven-dried glassware under a dry nitrogen atmosphere. All reagents were purchased as reagent grade and used without further purification. Dimethyl formamide was degassed and dried using an LC Technical SP-1 solvent purification system. Ethanol was distilled over Mg(OEt)₂. Ethyl acetate, methanol, and petroleum ether were distilled prior to use. All other solvents were used as received unless stated otherwise. Solid Phase Extraction (SPE) was performed using Strata Cis E 70 Å, 55 μm 20 g/60 mL columns. RP-HPLC was performed with an Agilent 1100 using a Jupiter C18 300 Å, 5 μm, 2.0 mm×250 mm column at a flow rate of 0.2 mLmin⁻¹ with a DAD Detector operating at 262, 280 and 320 nm. A suitably adjusted gradient of 5% B to 100% B was used, where solvent A was 0.1% HCOOH in H₂O and B was 20% A in MeCN. Flash chromatography was carried out using 0.063-0.1 mm silica gel with the desired solvent. Thin layer chromatography (TLC) was performed using 0.2 mm Kieselgel F254 (Merck) silica plates and compounds were visualised using UV irradiation at 254 or 365 nm and/or staining with a solution of potassium permanganate and potassium carbonate in aqueous sodium hydroxide. Preparative TLC was performed using 500 μm, 20×20 cm Uniplate™ (Analtech) silica gel TLC plates and compounds were visualised using UV irradiation at 254 or 365 nm. Melting points were determined on a Kofler hot-stage apparatus and are uncorrected. Infrared spectra were obtained using a Perkin-Elmer Spectrum 100 FTIR spectrometer on a film ATR sampling accessory. Absorption maxima are expressed in wavenumbers (cm⁻¹). NMR spectra were recorded as indicated on either a Bruker Avance 400 spectrometer operating at 400 MHz for 1H nuclei and 100 MHz for ¹³C nuclei, a Bruker DRX-400 spectrometer operating at 400 MHz for 1H nuclei and 100 MHz for ¹³C nuclei, a Bruker Avance AVIII-HD 500 spectrometer operating at 500 MHz for ¹H nuclei and 125 MHz for ¹³C nuclei or a Bruker Avance 600 spectrometer operating at 600 MHz for 1H nuclei and 150 MHz for ¹³C nuclei. 1H and ¹³C chemical shifts are reported in parts per million (ppm) relative to CDCl₃ (¹H and ¹³C) or (CD₃)₂SO (¹H and ¹³C). ¹⁵N chemical shifts were referenced using the unified scale (Harris et al., 2008) as implemented by the Bruker library function “xiref.” ¹H NMR data is reported as chemical shift, relative integral, multiplicity (s, singlet; assignment). Assignments were made with the aid of COSY, NOESY, HSQC and HMBC experiments where required. High resolution mass spectra were recorded on a Bruker micrOTOF-Q II mass spectrometer with ESI ionisation source. Ultraviolet-visible spectra were run as H₂O solutions on a Shimadzu UV-2101PC scanning spectrophotometer.

In one embodiment, the invention provides a composition comprising 3,6,7-trimethyllumazine for use in the methods described above. In one embodiment, the composition comprises a therapeutically effective amount of 3,6,7-trimethyllumazine.

In one embodiment of the invention, the composition comprising 3,6,7-trimethyllumazine comprises honey. In one particular embodiment, the composition comprising 3,6,7-trimethyllumazine consists of honey.

In one embodiment, the honey is of a floral origin substantially from the genus Leptospermum. In one embodiment, the honey is substantially from plants selected from the group consisting of: Leptospermum scoparium, Leptospermum polygalifolium, Leptospermum subtenue, and/or combinations thereof.

In one embodiment, the composition comprises about 2.5 μg/mL to about 80 μg/mL 3,6,7-trimethyllumazine. In one embodiment, the composition comprises about 2.5 μg/mL, about 5 μg/mL, about 10 μg/mL, about 20 μg/mL, about 40 μg/mL, about 50 μg/mL, about 60 μg/mL, about 70 μg/mL or about 80 μg/mL 3,6,7-trimethyllumazine, or the composition comprises a concentration of 3,6,7-trimethyllumazine of 2.5 μg/mL to 5 μg/mL, 5 μg/mL to 10 μg/mL, 10 μg/mL to 20 μg/mL, 20 μg/mL to 40 μg/mL, 40 μg/mL to 50 μg/mL, 50 μg/mL to about 60 μg/mL, 60 μg/mL to 70 μg/mL or 70 μg/mL to 80 μg/mL 3,6,7-trimethyllumazine.

In one embodiment, the composition comprises about 5 to about 80 mg/kg 3,6,7-trimethyllumazine. In one embodiment, the composition comprises about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 70 mg/kg or about 80 mg/kg of 3,6,7-trimethyllumazine or the composition comprises a concentration of 3,6,7-trimethyllumazine of 5 to 10 mg/kg, 10 to 15 mg/kg, 15 to 20 mg/kg, 20 to 25 mg/kg, 25 to 30 mg/kg, 30 to 35 mg/kg, 35 to 40 mg/kg, 40 to 45 mg/kg, 45 to 50 mg/kg, 50 to 55 mg/kg, 55 to 60 mg/kg, 60 to 70 mg/kg or 70 to 80 mg/kg.

In one embodiment, the honey is raw honey. In one embodiment, the honey is heat-treated or pasteurised according to methods that would be well known to a person skilled in the art.

In one particular embodiment, the composition comprises a honey extract. In one embodiment, the composition consists of a honey extract.

In one embodiment, the honey extract comprises a concentration of 3,6,7-trimethyllumazine that is higher than the concentration of 3,6,7-trimethyllumazine found naturally occurring in honey.

In one embodiment, the honey extract comprises a concentration of 3,6,7-trimethyllumazine that is higher than the concentration of 3,6,7-trimethyllumazine found naturally occurring in the honey from which the extract was derived.

In one embodiment, the honey from which the extract is derived is of a floral origin substantially from the genus Leptospermum. In one embodiment, the honey from which the extract is derived is substantially from plants selected from the group consisting of: Leptospermum scoparium, Leptospermum polygalifolium, Leptospermum subtenue, and/or combinations thereof.

In one embodiment, the extract comprises about 2.5 μg/mL to about 1000 μg/mL 3,6,7-trimethyllumazine. In one embodiment, the extract comprises about 2.5 μg/mL, about 5 μg/mL, about 10 μg/mL, about 20 μg/mL, about 40 μg/mL, about 50 μg/mL, about 60 μg/mL, about 70 μg/mL, about 80 μg/mL, about 90 μg/mL, about 100 μg/mL, 150 μg/mL, about 200 μg/mL, about 250 μg/mL, about 300 μg/mL, about 350 μg/mL, about 400 μg/mL, about 450 about 500 μg/mL, about 550 μg/mL, about 600 μg/mL, about 650 μg/mL, about 700 μg/mL, about 750 μg/mL, about 800 μg/mL, about 850 μg/mL, about 900 μg/mL or about 950 μg/mL, to about 1000 3,6,7-trimethyllumazine, or the composition comprises 3,6,7-trimethyllumazine about 2.5 to 5 μg/mL, about 5 to 10 μg/mL, about 10 to 20 μg/mL, about 20 to 40 μg/mL, about 40 to 50 μg/mL, about 50 to 60 μg/mL, about 60 to 70 μg/mL, about 70 to 80 μg/mL, about 80 to 90 μg/mL, about 90 to 100 μg/mL, about 100 to 150 μg/mL, 150 to 200 μg/mL, about 200 to 250 μg/mL, about 250 to 300 μg/mL, about 300 to 350 μg/mL, about 350 to 400 μg/mL, about 400 to 450 μg/mL, about 450 to 500 μg/mL, about 500 to 550 μg/mL, about 550 to 600 μg/mL, about 600 to 650 μg/mL, about 650 to 700 μg/mL, about 700 to 750 μg/mL, about 750 to 800 μg/mL, about 800 to 850 μg/mL, about 850 to 900 μg/mL, about 900 to 950 μg/mL or about 950 to 1000 μg/mL.

In one embodiment, the extract comprises 3,6,7-trimethyllumazine about 5 to about 3000 mg/kg. In one embodiment, the extract comprises about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 70 mg/kg, about 80 mg/kg, about 90 mg/kg, about 100 mg/kg, about 150 mg/kg, about 200 mg/kg, about 250 mg/kg, about 300 mg/kg, about 350 mg/kg, about 400 mg/kg, about 450 mg/kg, about 500 mg/kg, about 550 mg/kg, about 600 mg/kg, about 650 mg/kg, about 700 mg/kg, about 750 mg/kg, about 800 mg/kg, about 850 mg/kg, about 900 mg/kg, about 950 mg/kg, about 1000 mg/kg, about 1100 mg/kg, about 1200 mg/kg, about 1300 mg/kg, about 1400 mg/kg, about 1500 mg/kg, about 1600 mg/kg, about 1700 mg/kg, about 1800 mg/kg, about 1900 mg/kg, about 2000 mg/kg, about 2100 mg/kg, about 2200 mg/kg, about 2300 mg/kg, about 2400 mg/kg, about 2500 mg/kg, about 2600 mg/kg, about 2700 mg/kg, about 2800 mg/kg or about 2900 mg/kg to about 3000 mg/kg, of 3,6,7-trimethyllumazine or the extract comprises a concentration of 3,6,7-trimethyllumazine of 5 to 10 mg/kg, 10 to 15 mg/kg, about 15 to 20 mg/kg, about 20 to 25 mg/kg, about 25 to 30 mg/kg, about 30 to 35 mg/kg, about 35 to 40 mg/kg, or about 40 to 45 mg/kg, about 45 to 50 mg/kg, about 50 to 55 mg/kg, about 55 to 60 mg/kg, about 60 to 70 mg/kg or about 70 to 80 mg/kg, about 90 to 100 mg/kg, about 100 to 150 mg/kg, about 150 to 200 mg/kg, about 200 mg/kg, about 250 to 300 mg/kg, about 300 to 350 mg/kg, about 350 to 400 mg/kg, about 400 to 450 mg/kg, about 450 to 500 mg/kg, about 500 to 550 mg/kg, about 550 to 600 mg/kg, about 600 to 650 mg/kg, about 650 to 700 mg/kg, about 700 to 750 mg/kg, about 750 to 800 mg/kg, about 800 to 850 mg/kg, about 850 to 900 mg/kg, about 900 to 950 mg/kg, about 950 to 1000 mg/kg, about 1000 to 1100 mg/kg, about 1100 to 1200 mg/kg, about 1200 to 1300 mg/kg, about 1300 to 1400 mg/kg, about 1400 to 1500 mg/kg, about 1500 to 1600 mg/kg, about 1600 to 1700 mg/kg, about 1700 to 1800 mg/kg, about 1800 to 1900 mg/kg, about 1900 to 2000 mg/kg, about 2000 to 2100 mg/kg, about 2100 to 2200 mg/kg, about 2200 to 2300 mg/kg, about 2300 to 2400 mg/kg, about 2400 to 2500 mg/kg, about 2500 to 2600 mg/kg, about 2600 to 2700 mg/kg, about 2700 to 2800 mg/kg, about 2800 to 2900 mg/kg or about 2900 to 3000 mg/kg.

In one embodiment, the composition comprises at least 0.1%, 1%, 5%, 10%, 20%, 30%, 40%, 50%, 60%, 70%, 80% or 90% 3,6,7-trimethyllumazine, or comprises substantially pure 3,6,7-trimethyllumazine.

In one embodiment, the composition comprises a honey extract and further comprises honey.

In one embodiment, the composition comprises isolated 3,6,7-trimethyllumazine that is isolated from honey. In one embodiment, the honey is of a floral origin substantially from the genus Leptospermum. In one embodiment, the honey is substantially from plants selected from the group consisting of: Leptospermum scoparium, Leptospermum polygalifolium, Leptospermum subtenue, and/or combinations thereof.

The 3,6,7-trimethyllumazine may be isolated by any method well known to a person skilled in the art. In one embodiment, the 3,6,7-trimethyllumazine is isolated by subjecting of the honey to SPE (solid phase extraction), followed by normal-phase flash chromatography and preparative TLC (thin layer chromatography).

In one embodiment, the 3,6,7-trimethyllumazine is isolated by a method as described in the applicant's earlier patent published as NZ 722140, and as shown below.

Chemical Isolation of 3,6,7-trimethyllumazine

Raw manuka honey (51.3 g) was dissolved in H₂O+0.1% HCOOH (150 mL) and sonicated for 20 min. The resulting suspension was filtered through Celite and the filtrate used in the next step.

The filtrate was divided into two portions of 100 mL and each portion was subjected to SPE using MeOH—H₂O+0.1% HCOOH (1:9, 80 mL) to remove undesired substances. The desired fraction was then eluted using MeOH—H₂O+0.1% HCOOH (4:1, 80 mL). The two fractions were combined and concentrated to give the crude extract (0.23 g) which was further purified by flash chromatography (pet. ether-EtOAc 1:4) to give purified extract (3 mg) as a brown solid.

Several purified extracts were combined (6 mg total) and further purified by preparative TLC (pet. ether-EtOAc 1:3, 4 runs) to give 3 (4 mg) (as shown below) as a colourless solid.

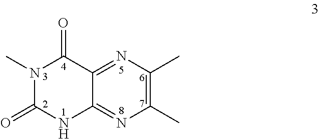

While using HPLC to examine New Zealand and Australian honeys derived from species of Leptospermum, Eucalyptus, Kunzea and Knightia for the presence of leptosperin (4) (Kato et al. 2012 and 2014; Aitken, et al. 2013; structure as shown below) a proposed biomarker for Leptospermum honey, an unexpected UV absorbance was noted at 320 nm.

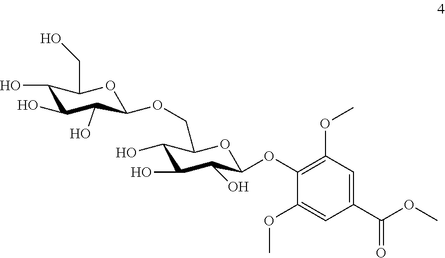

This peak was observed only in Leptospermum honeys (L. scoparium, L. scoparium var. exinium, L. polygalifolium, L. subtenue), including honey derived from L. subtenue in which no leptosperin was detected. The use of solid phase extraction (SPE) followed by reverse-phase HPLC enabled purification of the compound that exhibited the UV absorbance at 320 nm. However this method was time consuming, low yielding and not scalable, hence a more efficient isolation method was sought. Subjection of manuka honey to SPE, followed by normal-phase flash chromatography and preparative TLC enabled isolation of 3,6,7-trimethyllumazine as a colourless solid in sufficient quantity to conduct spectroscopic analysis.

In one embodiment of the invention, the composition comprises synthetic 3,6,7-trimethyllumazine or isolated 3,6,7-trimethyllumazine. In one embodiment, the composition further comprises honey. In one embodiment, the composition consists of synthetic 3,6,7-trimethyllumazine and honey. In one embodiment, the composition consists of isolated 3,6,7-trimethyllumazine and honey.

In one embodiment, the honey is of a floral origin substantially from the genus Leptospermum. In one embodiment, the honey is substantially from plants selected from the group consisting of: Leptospermum scoparium, Leptospermum polygalifolium, Leptospermum subtenue, and/or combinations thereof.

In one embodiment, the composition comprises synthetic 3,6,7-trimethyllumazine or isolated 3,6,7-trimethyllumazine about 2.5 μg/mL to about 1000 μg/mL 3,6,7-trimethyllumazine. In one embodiment, the composition comprises synthetic 3,6,7-trimethyllumazine or isolated 3,6,7-trimethyllumazine from about 2.5 μg/mL, about 5 μg/mL, about 10 μg/mL, about 20 μg/mL, about 40 μg/mL, about 50 μg/mL, about 60 μg/mL, about 70 μg/mL, about 80 μg/mL, about 90 μg/mL, about 100 μg/mL, 150 μg/mL, about 200 μg/mL, about 250 μg/mL, about 300 μg/mL, about 350 μg/mL, about 400 μg/mL, about 450 about 500 μg/mL, about 550 μg/mL, about 600 μg/mL, about 650 μg/mL, about 700 μg/mL, about 750 μg/mL, about 800 μg/mL, about 850 μg/mL, about 900 μg/mL or about 950 μg/mL, to about 1000 μg/mL or the composition comprises synthetic 3,6,7-trimethyllumazine or isolated 3,6,7-trimethyllumazine of about 2.5 to 5 μg/mL, about 5 to 10 μg/mL, about 10 to 20 μg/mL, about 20 to 40 μg/mL, about 40 to 50 μg/mL, about 50 to 60 μg/mL, about 60 to 70 μg/mL, about 70 to 80 μg/mL, about 80 to 90 μg/mL, about 90 to 100 μg/mL, about 100 to 150 μg/mL, 150 to 200 μg/mL, about 200 to 250 μg/mL, about 250 to 300 μg/mL, about 300 to 350 μg/mL, about 350 to 400 μg/mL, about 400 to 450 μg/mL, about 450 to 500 μg/mL, about 500 to 550 μg/mL, about 550 to 600 μg/mL, about 600 to 650 μg/mL, about 650 to 700 μg/mL, about 700 to 750 μg/mL, about 750 to 800 μg/mL, about 800 to 850 μg/mL, about 850 to 900 μg/mL, about 900 to 950 μg/mL or about 950 to 1000 μg/mL.

In one embodiment, the composition comprises synthetic 3,6,7-trimethyllumazine or isolated 3,6,7-trimethyllumazine from about 5 mg/kg to about 3000 mg/kg. In one embodiment, the composition comprises synthetic 3,6,7-trimethyllumazine or isolated 3,6,7-trimethyllumazine from about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 70 mg/kg, about 80 mg/kg, about 90 mg/kg, about 100 mg/kg, about 150 mg/kg, about 200 mg/kg, about 250 mg/kg, about 300 mg/kg, about 350 mg/kg, about 400 mg/kg, about 450 mg/kg, about 500 mg/kg, about 550 mg/kg, about 600 mg/kg, about 650 mg/kg, about 700 mg/kg, about 750 mg/kg, about 800 mg/kg, about 850 mg/kg, about 900 mg/kg, about 950 mg/kg, about 1000 mg/kg, about 1100 mg/kg, about 1200 mg/kg, about 1300 mg/kg, about 1400 mg/kg, about 1500 mg/kg, about 1600 mg/kg, about 1700 mg/kg, about 1800 mg/kg, about 1900 mg/kg, about 2000 mg/kg, about 2100 mg/kg, about 2200 mg/kg, about 2300 mg/kg, about 2400 mg/kg, about 2500 mg/kg, about 2600 mg/kg, about 2700 mg/kg, about 2800 mg/kg or about 2900 mg/kg to about 3000 mg/kg, or the composition comprises synthetic 3,6,7-trimethyllumazine or isolated 3,6,7-trimethyllumazine of about 5 to 10 mg/kg, about 10 to 15 mg/kg, about 15 to 20 mg/kg, about 20 to 25 mg/kg, about 25 to 30 mg/kg, about 30 to 35 mg/kg, about 35 to 40 mg/kg, about 40 to 45 mg/kg, about 45 to 50 mg/kg, about 50 to 55 mg/kg, about 55 to 60 mg/kg, about 60 to 70 mg/kg or about 70 to 80 mg/kg, about 90 to 100 mg/kg, about 100 to 150 mg/kg, about 150 to 200 mg/kg, about 200 mg/kg, about 250 to 300 mg/kg, about 300 to 350 mg/kg, about 350 to 400 mg/kg, about 400 to 450 mg/kg, about 450 to 500 mg/kg, about 500 to 550 mg/kg, about 550 to 600 mg/kg, about 600 to 650 mg/kg, about 650 to 700 mg/kg, about 700 to 750 mg/kg, about 750 to 800 mg/kg, about 800 to 850 mg/kg, about 850 to 900 mg/kg, about 900 to 950 mg/kg, about 950 to 1000 mg/kg, about 1000 to 1100 mg/kg, about 1100 to 1200 mg/kg, about 1200 to 1300 mg/kg, about 1300 to 1400 mg/kg, about 1400 to 1500 mg/kg, about 1500 to 1600 mg/kg, about 1600 to 1700 mg/kg, about 1700 to 1800 mg/kg, about 1800 to 1900 mg/kg, about 1900 to 2000 mg/kg, about 2000 to 2100 mg/kg, about 2100 to 2200 mg/kg, about 2200 to 2300 mg/kg, about 2300 to 2400 mg/kg, about 2400 to 2500 mg/kg, about 2500 to 2600 mg/kg, about 2600 to 2700 mg/kg, about 2700 to 2800 mg/kg, about 2800 to 2900 mg/kg or about 2900 to 3000 mg/kg.

In one embodiment, the composition comprises 0.1% to 100% 3,6,7-trimethyllumazine. In one embodiment, the composition comprises at least 0.1%, 1%, 5%, 10%, 20%, 30%, 40%, 50%, 60%, 70%, 80% or 90% 3,6,7-trimethyllumazine, or comprises substantially pure 3,6,7-trimethyllumazine.

Compositions comprising honey-derived 3,6,7-trimethyllumazine and/or synthetic 3,6,7-trimethyllumazine are not anticipated to have side effects. 3,6,7-Trimethyllumazine is naturally occurring in some honey and such honey containing 3,6,7-trimethyllumazine has been sold and consumed for many years.

The composition comprising 3,6,7-trimethyllumazine may be formulated as a medicament, therapeutic product or health supplement.

The composition comprising 3,6,7-trimethyllumazine is formulated into a range of delivery systems, including but not limited to, liquid formulations, capsules, fast moving consumer goods, chewable tablets, tablets, suppositories, intravenous preparations, intramuscular preparations, subcutaneous preparations, solutions, food, beverages, dietary supplements, cosmetic formulations, gels, lotions, powders or sprays.

In one particular embodiment, the method of the invention as described above comprises administration of the composition comprising about 1 mg to about 3000 mg 3,6,7-trimethyllumazine. In one particular embodiment, the method of the invention as described above comprises administration of the composition comprising about 1 mg, 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg or 3000 mg 3,6,7-trimethyllumazine.

In one particular embodiment, the method of the invention as described above comprises administration of composition comprising 3,6,7-trimethyllumazine, including wherein the composition is honey or a honey extract. In one embodiment, the honey in the method of the invention is administered at a dose of about 5 g to about 100 g. In one embodiment, the honey is administered at a dose of about 5 g, 10 g, 15 g, 20 g, 25 g, 30 g, 40 g, 50 g, 60 g, 70 g, 80 g, 90 g or 100 g. In one embodiment, the honey is administered at a dose of equivalent to about 1 teaspoon to about 5 tablespoons of honey. In one embodiment, the honey is administered as a single dose or in multiple doses.

In one embodiment, the composition comprising 3,6,7-trimethyllumazine is administered as a single dose or as a divided dose. In one embodiment, the composition comprising 3,6,7-trimethyllumazine is administered as one, two, three or four separate doses.

In one particular embodiment, the method of the invention as described above comprises administration of the composition comprising 3,6,7-trimethyllumazine one, two, three or four times daily. In another embodiment, the method of the invention as described above comprises administration of the composition comprising 3,6,7-trimethyllumazine one, two, three, four, five, six or seven times weekly.

The concentration of 3,6,7-trimethyllumazine can vary significantly from honey sample to honey sample. Therefore, in one particular embodiment of the invention described herein, the composition comprising honey has a standardised concentration of 3,6,7-trimethyllumazine.

In one embodiment, the composition comprising 3,6,7-trimethyllumazine has a standardised concentration of 3,6,7-trimethyllumazine obtained by:
selecting a first composition with a known concentration of 3,6,7-trimethyllumazine;
selecting at least one further composition with a known concentration of 3,6,7-trimethyllumazine; and
combining the first composition with the second composition to obtain a final composition with a standardised 3,6,7-trimethyllumazine concentration of about 5 mg/kg to about 3000 mg/kg.

In one embodiment, the composition comprising 3,6,7-trimethyllumazine has a standardised concentration of 3,6,7-trimethyllumazine obtained by:
selecting a first composition with a known concentration of 3,6,7-trimethyllumazine; and
combining the selected first composition with one or more of:
   synthetic 3,6,7-trimethyllumazine;
   isolated 3,6,7-trimethyllumazine;
   a honey extract comprising 3,6,7-trimethyllumazine; and/or
   3,6,7-trimethyllumazine derived directly from a plant of the genus Leptospermum;
to form a composition with a standardised 3,6,7-trimethyllumazine concentration of about 5 mg/kg to about 3000 mg/kg.

In one embodiment, the composition comprising 3,6,7-trimethyllumazine has a standardised concentration of 3,6,7-trimethyllumazine obtained by:
selecting a first composition comprising honey with a known concentration of 3,6,7-trimethyllumazine; and
combining the selected first composition comprising honey with one or more of:
   synthetic 3,6,7-trimethyllumazine;
   isolated 3,6,7-trimethyllumazine; and
   a honey extract comprising 3,6,7-trimethyllumazine; and/or
   3,6,7-trimethyllumazine derived directly from a plant of the genus Leptospermum;
to form a composition with a standardised 3,6,7-trimethyllumazine concentration of about 5 to about 3000 mg/kg.

In one embodiment, the composition comprises honey, a honey extract, isolated 3,6,7-trimethyllumazine and/or synthetic 3,6,7-trimethyllumazine.

In one embodiment, the 3,6,7-trimethyllumazine derived directly from a plant is derived directly from the flowers, nectar, roots, fruit, seeds, bark, oil, leaves, wood, stems or other plant material of a plant of the genus Leptospermum.

In one embodiment, the standardised 3,6,7-trimethyllumazine concentration is about 2.5 μg/mL to about 1000 μg/mL 3,6,7-trimethyllumazine. In one embodiment, the standardised 3,6,7-trimethyllumazine concentration is from: about 2.5 μg/mL, about 5 μg/mL, about 10 μg/mL, about 20 μg/mL, about 40 μg/mL, about 50 μg/mL, about 60 μg/mL, about 70 μg/mL, about 80 μg/mL, about 90 μg/mL, about 100 μg/mL, 150 μg/mL, about 200 μg/mL, about 250 μg/mL, about 300 μg/mL, about 350 μg/mL, about 400 μg/mL, about 450 about 500 μg/mL, about 550 μg/mL, about 600 μg/mL, about 650 μg/mL, about 700 μg/mL, about 750 μg/mL, about 800 μg/mL, about 850 μg/mL, about 900 μg/mL or about 950 μg/mL, to about 1000 3,6,7-trimethyllumazine.

In one embodiment, the standardised 3,6,7-trimethyllumazine concentration is: about 5 mg/kg to about 3000 mg/kg. In one embodiment, the standardised 3,6,7-trimethyllumazine concentration is from: about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 70 mg/kg, about 80 mg/kg, about 90 mg/kg, about 100 mg/kg, about 150 mg/kg, about 200 mg/kg, about 250 mg/kg, about 300 mg/kg, about 350 mg/kg, about 400 mg/kg, about 450 mg/kg, about 500 mg/kg, about 550 mg/kg, about 600 mg/kg, about 650 mg/kg, about 700 mg/kg, about 750 mg/kg, about 800 mg/kg, about 850 mg/kg, about 900 mg/kg, about 950 mg/kg, about 1000 mg/kg, about 1100 mg/kg, about 1200 mg/kg, about 1300 mg/kg, about 1400 mg/kg, about 1500 mg/kg, about 1600 mg/kg, about 1700 mg/kg, about 1800 mg/kg, about 1900 mg/kg, about 2000 mg/kg, about 2100 mg/kg, about 2200 mg/kg, about 2300 mg/kg, about 2400 mg/kg, about 2500 mg/kg, about 2600 mg/kg, about 2700 mg/kg, about 2800 mg/kg or about 2900 mg/kg to about 3000 mg/kg, of 3,6,7-trimethyllumazine.

In one embodiment, the concentration of the 3,6,7-trimethyllumazine is determined by chromatography, analytical measurements, spectrophotometry and/or any other method known to a person skilled in the art. In one embodiment, the concentration of 3,6,7-trimethyllumazine is determined by reverse-phase HPLC system.

In one embodiment, the 3,6,7-trimethyllumazine concentration in the honey is determined by a method as previously described in NZ 722140.

In another particular aspect, the invention provides a method of making a composition with anti-inflammatory, analgesic and/or TG2, JAK, and/or COX-2 inhibitory activity comprising:
a. testing a first composition comprising honey for 3,6,7-trimethyllumazine concentration;
b. testing at least one further composition comprising honey for 3,6,7-trimethyllumazine concentration;
c. selecting a composition comprising honey with a 3,6,7-trimethyllumazine concentration greater than about 5 mg/kg 3,6,7-trimethyllumazine;
d. selecting at least one further composition comprising honey with a 3,6,7-trimethyllumazine concentration greater than about 5 mg/kg 3,6,7-trimethyllumazine; and
e. combining the selected composition comprising honey to form a honey composition with a 3,6,7-trimethyllumazine concentration of about 5 to about 80 mg/kg.

In one embodiment, the compositions comprising honey are selected if they have a concentration of 3,6,7-trimethyllumazine greater than: about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 70 mg/kg or about 80 mg/kg.

In one embodiment, the method further comprises a step of packaging the composition identified as having anti-inflammatory activity with a label identifying that it has a 3,6,7-trimethyllumazine concentration of about 5 to about 80 mg/kg. In one particular embodiment about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 70 mg/kg or about 80 mg/kg 3,6,7-trimethyllumazine. In one embodiment, 5 to 10 mg/kg, about 10 to 15 mg/kg, about 15 to 20 mg/kg, about 20 to 25 mg/kg, about 25 to 30 mg/kg, about 30 to 35 mg/kg, about 35 to 40 mg/kg, or about 40 to 45 mg/kg, about 45 to 50 mg/kg, about 50 to 55 mg/kg, about 55 to 60 mg/kg, 60 to 70 mg/kg or about 70 to 80 mg/kg 3,6,7-trimethyllumazine.

In one particular embodiment, the composition is honey or a honey extract.

In one embodiment, the composition with anti-inflammatory activity is suitable for use in any one of the methods as described above and below.

In one embodiment, the concentration of the 3,6,7-trimethyllumazine is determined by chromatography, analytical measurements, spectrophotometry and/or any other method known to a person skilled in the art. In one embodiment, the concentration of 3,6,7-trimethyllumazine is determined by reverse-phase HPLC.

In one embodiment, the 3,6,7-trimethyllumazine concentration is determined by a method as previously described in NZ 722140.

In another particular aspect, the invention provides a method of identifying a composition as having anti-inflammatory, analgesic and/or TG2, JAK, and/or COX-2 inhibitory activity comprising:
a. testing a composition for 3,6,7-trimethyllumazine concentration; and
   i. identifying the composition as having anti-inflammatory, analgesic and/or TG2, JAK, and/or COX-2 inhibitory activity if it contains a 3,6,7-trimethyllumazine concentration greater than about 5 to about 80 mg/kg 3,6,7-trimethyllumazine; or
   ii. identifying the composition as not having anti-inflammatory, analgesic and/or TG2, JAK, and/or COX-2 inhibitory activity if it contains a 3,6,7-trimethyllumazine concentration lower than about 5 mg/kg 3,6,7-trimethyllumazine.

In one embodiment, the composition comprises honey, a honey extract, isolated 3,6,7-trimethyllumazine and/or synthetic 3,6,7-trimethyllumazine.

In one embodiment, the composition is determined as having anti-inflammatory activity if it contains greater than about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 70 mg/kg or about 80 mg/kg 3,6,7-trimethyllumazine.

In one embodiment, the method further comprises a step of packaging the composition identified as having anti-inflammatory activity with a label identifying that it has a 3,6,7-trimethyllumazine concentration of about 5 to about 80 mg/kg and as having anti-inflammatory activity.

In one embodiment, the composition with anti-inflammatory activity is suitable for use in any one of the methods as described above and below.

In one particular embodiment, the composition is honey or a honey extract.

In another particular aspect, the invention provides a method of identifying a composition with anti-inflammatory, analgesic and/or TG2, JAK, and/or COX-2 inhibitory activity suitable for use in a method of preventing, ameliorating or treating a condition associated with inflammation comprising:
a. testing a composition for 3,6,7-trimethyllumazine concentration; and
   i. identifying the composition as suitable for use in a method of preventing, ameliorating or treating a condition associated with inflammation of the gastrointestinal tract if it contains a 3,6,7-trimethyllumazine concentration greater than about 5 to about 80 mg/kg 3,6,7-trimethyllumazine; or
   ii. identifying the composition as not suitable for use in a method of preventing, ameliorating or treating a condition associated with inflammation of the gastrointestinal tract if it contains a 3,6,7-trimethyllumazine concentration lower than about 5 mg/kg 3,6,7-trimethyllumazine.

In one embodiment, the composition comprises honey, a honey extract, isolated 3,6,7-trimethyllumazine and/or synthetic 3,6,7-trimethyllumazine.

In one embodiment, the condition associated with inflammation is a COX-2 associated condition, a TG2 associated condition and/or a JAK-associated condition.

In one embodiment, the COX-2 associated condition is selected from the group consisting of gastrointestinal inflammatory diseases, gastric ulcers (such as peptic ulcers), gastritis, inflammatory bowel disease (IBD), Crohn's disease, ulcerative colitis, irritable bowel syndrome (IBS), digestive disease, gastroesophageal reflux disease (GERD), heartburn, acid reflux, Helicobacter pylori infection, mouth ulcers, stomatitis, pharyngitis, gingivitis, esophageal ulcers, inflammatory and degenerative nervous system disorders, neuropsychiatric illnesses (such as schizophrenia and bipolar mood disorder), neurodegenerative disorders (such as traumatic brain injury, multiple sclerosis and Alzheimer's disease), nervous system disorders such as Parkinson's disease and/or seizures, brain hypoxia/ischemia, Creutzfeldt-Jakob disease, amyotrophic lateral sclerosis, arthritis (such as rheumatoid arthritis, juvenile rheumatoid arthritis and ankylosing spondylitis), chronic inflammation, cardiovascular diseases, pain (such as, acute pain (such as pain caused by a physical injury), chronic pain and dysmenorrhea (pain associated with menstruation)), cancer (such as colorectal cancer (CRC)) and musculoskeletal diseases.

In one embodiment, the TG2 associated condition is selected from the group consisting of gastrointestinal inflammatory diseases, gastric ulcers (such as peptic ulcers), gastritis, TG2 associated inflammatory conditions, inflammatory bowel disease (IBD), Crohn's disease, ulcerative colitis, irritable bowel syndrome (IBS), digestive diseases, gastroesophageal reflux disease (GERD), heartburn, acid reflux, Helicobacter pylori infection, mouth ulcers, stomatitis, pharyngitis, gingivitis and/or esophageal ulcers, neuropsychiatric illnesses (such as schizophrenia and bipolar mood disorder), multiple sclerosis, neurodegenerative disorders (such as traumatic brain injury, multiple sclerosis, and Alzheimer's disease), cardiovascular diseases, cancer, arthritis, celiac disease, Huntington's disease, fibrosis and cancer. TG2 also plays a role in wound healing.

In one embodiment, the JAK associated condition is selected from the group consisting of gastrointestinal inflammatory diseases, gastric ulcers (such as peptic ulcers), gastritis, JAK associated inflammatory conditions, inflammatory bowel disease (IBD), Crohn's disease, ulcerative colitis, irritable bowel syndrome (IBS), digestive diseases, gastroesophageal reflux disease (GERD), heartburn, acid reflux, Helicobacter pylori infection, mouth ulcers, stomatitis, pharyngitis, gingivitis and/or esophageal ulcers, neuropsychiatric illnesses (such as schizophrenia and bipolar mood disorder), multiple sclerosis, neurodegenerative disorders (such as traumatic brain injury, multiple sclerosis, and Alzheimer's disease), cardiovascular diseases, cancer, arthritis, chronic inflammation, auto-immune conditions, ulcerative colitis, alopecia areata, atopic dermatitis, diffuse scleroderma, Crohn's disease, vitiligo, hemophagocytic syndrome, non-infectious uveitis and cutaneous lupus erythematosus.

In one embodiment, the method further comprises a step of packaging the composition identified by the method above with a label identifying that it has a 3,6,7-trimethyllumazine concentration of about 5 to about 80 mg/kg.

In another particular aspect, the invention provides a method of identifying a composition with anti-inflammatory, analgesic and/or TG2, JAK, and/or COX-2 inhibitory activity suitable for use in a method of preventing, ameliorating or treating inflammation and/or pain comprising:
a. testing a batch of honey for 3,6,7-trimethyllumazine concentration; and
   i. identifying the composition as suitable for use in a method of preventing, ameliorating or treating inflammation and/or pain if it contains a 3,6,7-trimethyllumazine concentration greater than about 5 to about 80 mg/kg 3,6,7-trimethyllumazine; or
   ii. identifying the composition as not suitable for use in a method of preventing, ameliorating or treating inflammation and/or pain if it contains a 3,6,7-trimethyllumazine concentration lower than about 5 mg/kg 3,6,7-trimethyllumazine.

In one embodiment, the composition comprises honey, a honey extract, isolated 3,6,7-trimethyllumazine and/or synthetic 3,6,7-trimethyllumazine.

In one embodiment, the inflammation and/or pain is associated with a condition selected from TG2, JAK, and/or COX-2 associated conditions.

In one embodiment, the COX-2 associated condition is selected from the group consisting of gastrointestinal inflammatory diseases, gastric ulcers (such as peptic ulcers), gastritis, inflammatory bowel disease (IBD), Crohn's disease, ulcerative colitis, irritable bowel syndrome (IBS), digestive disease, gastroesophageal reflux disease (GERD), heartburn, acid reflux, Helicobacter pylori infection, mouth ulcers, stomatitis, pharyngitis, gingivitis, esophageal ulcers, inflammatory and degenerative nervous system disorders, neuropsychiatric illnesses (such as schizophrenia and bipolar mood disorder), neurodegenerative disorders (such as traumatic brain injury, multiple sclerosis and Alzheimer's disease), nervous system disorders such as Parkinson's disease and/or seizures, brain hypoxia/ischemia, Creutzfeldt-Jakob disease, amyotrophic lateral sclerosis, arthritis (such as rheumatoid arthritis, juvenile rheumatoid arthritis and ankylosing spondylitis), chronic inflammation, cardiovascular diseases, pain (such as, acute pain (such as pain caused by a physical injury), chronic pain and/or dysmenorrhea (pain associated with menstruation)), cancer (such as colorectal cancer (CRC)) and musculoskeletal diseases.

In one embodiment, the TG2 associated condition is selected from the group consisting of gastrointestinal inflammatory diseases, gastric ulcers (such as peptic ulcers), gastritis, TG2-associated inflammatory conditions, inflammatory bowel disease (IBD), Crohn's disease, ulcerative colitis, irritable bowel syndrome (IBS), digestive diseases, gastroesophageal reflux disease (GERD), heartburn, acid reflux, Helicobacter pylori infection, mouth ulcers, stomatitis, pharyngitis, gingivitis and/or esophageal ulcers, neuropsychiatric illnesses (such as schizophrenia and bipolar mood disorder), multiple sclerosis, neurodegenerative disorders (such as traumatic brain injury, multiple sclerosis, and Alzheimer's disease), cardiovascular diseases, cancer, arthritis, celiac disease, Huntington's disease, fibrosis, cancer and a wound.

In one embodiment, the JAK associated condition is selected from the group consisting of gastrointestinal inflammatory diseases, gastric ulcers (such as peptic ulcers), gastritis, JAK associated inflammatory conditions, inflammatory bowel disease (IBD), Crohn's disease, ulcerative colitis, irritable bowel syndrome (IBS), digestive diseases, gastroesophageal reflux disease (GERD), heartburn, acid reflux, Helicobacter pylori infection, mouth ulcers, stomatitis, pharyngitis, gingivitis and/or esophageal ulcers, neuropsychiatric illnesses (such as schizophrenia and bipolar mood disorder), multiple sclerosis, neurodegenerative disorders (such as traumatic brain injury, multiple sclerosis, and Alzheimer's disease), cardiovascular diseases, cancer, arthritis, chronic inflammation, auto-immune conditions, ulcerative colitis, alopecia areata, atopic dermatitis, diffuse scleroderma, Crohn's disease, vitiligo, hemophagocytic syndrome, non-infectious uveitis and cutaneous lupus erythematosus.

In one embodiment, the inflammation and/or pain is associated with the gastrointestinal tract.

In one embodiment, the method further comprises a step of packaging the composition identified by the method above with a label identifying that it has a 3,6,7-trimethyllumazine concentration of about 5 to about 80 mg/kg.

In one embodiment, the concentration of 3,6,7-trimethyllumazine may be determined by chromatography, analytical measurements, spectrophotometry and/or any other method known to a person skilled in the art. In one embodiment, the concentration of 3,6,7-trimethyllumazine is determined by reverse-phase HPLC.

### Quantification of 3,6,7-Trimethyllumazine in Manuka Honey Using Mass Spectrometry

In one embodiment of the invention, the 3,6,7-trimethyllumazine concentration is determined by a method as previously described in NZ 722140, and as shown below:

Described is a quantitative technique to measure 3,6,7-trimethyllumazine concentration using tandem mass spectrometry (LC-MS/MS). A heavier 3,6,7-trimethyllumazine isotope was synthesized and employed as an internal standard to compensate the matrix effect from manuka honey. There was no interference from endogenous compound in manuka honey and the 3 Da mass difference can be clearly distinguished on the mass spectrum. The results described further below of LC-MS/MS strongly correlates with previous data from HPLC quantification and fluorescence spectrometry. Therefore 3,6,7-trimethyllumazine can be accurately determined using all three methods. Results from LC-MS/MS quantification was comparatively lower than previous data from HPLC, this may be resulted from minor co-eluting compounds under the same HPLC peak. These findings demonstrate that quantitative mass spectrometry may be used as a standalone or complimentary approach for manuka honey authentication.

To validate the LC-MS/MS method, the mass spectrum of a typical manuka honey was obtained before and after the supplementation of the heavier 3,6,7-trimethyllumazine isotope. As shown, there was no significant interfering peaks from endogenous compounds in manuka honey from m/z 210-212. The 3 Da mass difference between the isotopes may be clearly identified on the mass spectrum. The final testing concentration of manuka honey was determined at 0.2% w/v to reduce sugar concentration while retaining relatively high mass spectrum resolution.

### LC-MS/MS Quantification

During the LC-stage, the endogenous 3,6,7-trimethyllumazine and the heavier isotope co-eluted at the exact same time (12.85 min). These isomers displayed almost identical MS/MS spectrum, while only differentiated by a 3 Da mass shift from m/z 189 to m/z 192. The most abundant common ion was observed at 148.05 m/z. The heavy isotopes were not present on the part of the structure represented by this fragment ion. This common ion is employed for 3,6,7-trimethyllumazine quantification to reduce background interference.

### Comparing LC-MS/MS and HPLC Quantification

Endogenous 3,6,7-trimethyllumazine concentration was quantified as 3-44 mg/kg using mass spectrometry quantification. The results demonstrated strong linear correlation with previous data from HPLC analysis on the same set of manuka honey samples (R2=0.9517). It should be noted that the mass spectrometry result was comparably lower than previous HPLC quantification (5-52 mg/kg). This suggests that other UV-absorbing compounds may have co-eluted with 3,6,7-trimethyllumazine under the same HPLC peak.

The results from mass spectrometry quantification also correlates well with the signature fluorescence at ₑₓ330 nm-ₑₘ470 nm (R²=0.8995).

Structure Elucidation of 3,6,7-trimethyllumazine

The chemical structure elucidation of 3,6,7-trimethyllumazine was described in NZ 722140, and as shown below.

TABLE 1
¹H, ¹³C and ¹⁵N NMR data for 3^{a}

| Position | δ_{C}/δ_{N}, type | δ_{H} | HMBC^{b} |
|---|---|---|---|
| 1 | NH | 8.42 br | |
| 2 | 149.9, C | | |
| 3 | 154.1, N | | |
| 4 | 161.1, C | | |
| 4a | 123.7, C | | |
| 5 | 292.0, N | | |
| 6 | 158.9, C | | |
| 7 | 150.6, C | | |
| 8 | 329.9, N | | |
| 8a | 144.8, C | | |
| 9 | 28.5, CH₃ | 3.50, s | 2, 3, 4 |
| 10 | 22.8, CH₃ | 2.63, s | 4a, 5, 6, 7 |
| 11 | 21.9, CH₃ | 2.67, s | 6, 7, 8 |

^{a1}H (400 MHz); ¹³C (100 MHz); ¹⁵N (60.8 MHz), chemical shift indirectly determined from ¹H-¹⁵N HMBC NMR data.
^{b}HMBC correlations are from protons stated to the indicated carbon or nitrogen.

Referring to Table 1 above, the molecular formula of the unknown compound was established as C₉H₁₀N₄O₂ by positive ion HRESIMS. The compound was soluble in CD₃OD and CDCl₃; the latter was used for recording NMR spectra due to the presence of a broad resonance at δ 8.55 ppm (H-1) that was not present in spectra recorded in CD₃₀D. This peak was assigned as an amide proton on the basis of its chemical shift and the absence of a distinctive hydroxyl absorption in the IR spectrum. Two singlets at δ 2.63 ppm (H-10) and δ 2.67 ppm (H-11) were assigned as heteroaryl methyl groups on the basis of their chemical shift, and the remaining singlet at δ 3.50 ppm (H-9) was assigned as an N-methyl group due to HMBC correlations of equal intensity to two quaternary carbonyl ¹³C signals (C-2, C-4, see below) and an HSQC correlation to a carbon signal at δ 28.5 ppm (C-9).

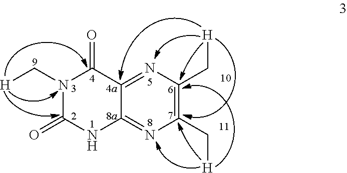 [SMILES: Cc1nc2[nH]c(=O)n(C)c(=O)c2nc1C]

¹H-¹⁵N HMBC correlations from H-10 and H-11 to N-5 and N-8 at δ 292.0 ppm and δ 329.9 ppm respectively, suggested that these two methyl groups were attached to a pyrazine ring. A 2,3-dimethyl substitution pattern was assigned based on ¹H-¹³C HMBC correlations from H-10 to C-7 and from H-11 to C-6.

Given the high degree of unsaturation in the structure and the presence of a pyrazine ring, a fused heterocyclic structure was proposed for the unknown compound. Furthermore, a similarity was noted between the chemical shifts of carbons C-2, C-4 and C-4a and shifts reported for analogous carbons in natural products containing lumazine structures (Pfleiderer, 1984; Kakoi, et al. 1995; Voerman, et al., 2005; Meyer, et al. 2010; Chen, et al. 2014). This observation, coupled with HMBC correlations from H-9 to C-2 and C-4 and an additional four bond coupling from H-10 to C-4a, led to the tentative assignment of the structure of the isolated compound as 3,6,7-trimethyllumazine (3). 3,6,7-Trimethyllumazine (3) was first synthesized in 1958 (Curran & Angier, 1958).

Since then it has been reported in several studies on related lumazines (Pfleiderer & Fink, 1963; Pfleiderer & Hutzenlaub, 1973; Ritzmann & Pfleiderer, 1973; Ram, et al. 1977; Southon & Pfleiderer, 1978; Uhlmann & Pfleiderer, 1981; Ram, et al. 1982; Bartke & Pfleiderer, 1989; Acuña-Cueva, et al. 2000). Characterisation data for lumazine 3 is limited to a melting point (Curran & Angier, 1958; Pfleiderer & Hutzenlaub, 1973), elemental analysis (Curran &Angier, 1958) and UV-vis peaks (Pfleiderer & Hutzenlaub, 1973; Ritzmann & Pfleiderer, 1973; Uhlmann & Pfleiderer, 1981); no NMR, MS or IR data have been reported to date.

The entire disclosures of all applications, patents and publications cited above and below, if any, are herein incorporated by reference.

For the avoidance of doubt, the term “composition” includes, but is not limited to, honey, honey extracts, or dried honey.

Reference to any prior art in this specification is not, and should not be taken as, an acknowledgement or any form of suggestion that that prior art forms part of the common general knowledge in the field of endeavour in any country in the world.

The invention may also be said broadly to consist in the parts, elements and features referred to or indicated in the specification of the application, individually or collectively, in any or all combinations of two or more of said parts, elements or features.

Wherein the foregoing description reference has been made to integers or components having known equivalents thereof, those integers are herein incorporated as if individually set forth.

It should be noted that various changes and modifications to the presently preferred embodiments described herein will be apparent to those skilled in the art.

Such changes and modifications may be made without departing from the spirit and scope of the invention and without diminishing its attendant advantages. It is therefore intended that such changes and modifications be included within the scope of the invention ass described by the appended claims.

### EXAMPLES

The above-identified compositions, medicaments, methods and uses are now described by reference to the Figures and specific Examples.

### Example 1—Fluorometric Assay

In this example, fluorometric inhibitor screening provides a rapid, sensitive and high throughput method to identify potential inhibitors of MMP-9.

### Methods and Materials

The MMP-9 inhibitor screening assay (fluorometric) kits were purchased from Abcam (Melbourne, Australia). The fluorometric kit contains the recombinant MMP-9 enzyme, MMP inhibitor NNGH (N-isobutyl-N-[4-methoxyphenylsulfonyl]glycyl hydroxamic acid), MMP fluorogenic substrate solubilised in DMSO, the fluorometric assay buffer and 96-well clear microplate.

Inhibitory activity on MMP-9 was assessed using the commercial MMP-9 inhibitor screening assay kit. MMP-9 activity was expressed as a change in fluorescence intensity measured using SpectraMax iD3 multi-mode microplate reader (Molecular Devices, San Jose, USA).

The assay employs a FRET-tagged (fluorescence resonance energy transfer) substrate, which can be hydrolysed by MMP-9 at a specific site (Abcam, 2018). The cleavage of the FRET substrate releases the quenched fluorescent Mca (7-methoxycoumarin-4-yl)-acetyl group (Abcam, 2018). The kit employs a quenched fluorogenic substrate Mca-Pro-Leu-Gly-Leu-Dpa-Ala-Arg-NH2, where the Mca fluorescence is quenched by Dpa until cleavage by MMPs. The amount of fluorescent product yielded by MMP-9 can be detected fluorometrically and it is proportional to the enzyme activity. Fluorescence were measured at ₑₓ320 nm-ₑₘ395 nm to minimise fluorescence interference from 3,6,7-trimethyllumazine at ₑₓ330 nm-ₑₘ470 nm. Assays were performed on a 96-well clear microplate included in the kit with a final reaction volume of 100 μL. Before adding the substrate, MMP-9 enzymes were incubated with testing samples and inhibitor control for 60 min at 37° C. The fluorescent substrate was added into each well prior to the assay to initiate the reaction. The assay was allowed to run for 20 min and the temperature in the reaction chamber was set to 37° C.

Testing samples were prepared comprising 3,6,7-trimethyllumazine prepared as outlined above.

A positive control was included with only MMP-9 and the fluorescent substrate, used as a reference to calculate the percentage inhibition. A broad spectrum MMP inhibitor NNGH was included as the negative control. A range of test controls were also included with 3,6,7-trimethyllumazine at the testing concentration without MMP-9 and the fluorescent substrate, which is essential to measure the autofluorescence generated by 3,6,7-trimethyllumazine.

### Results

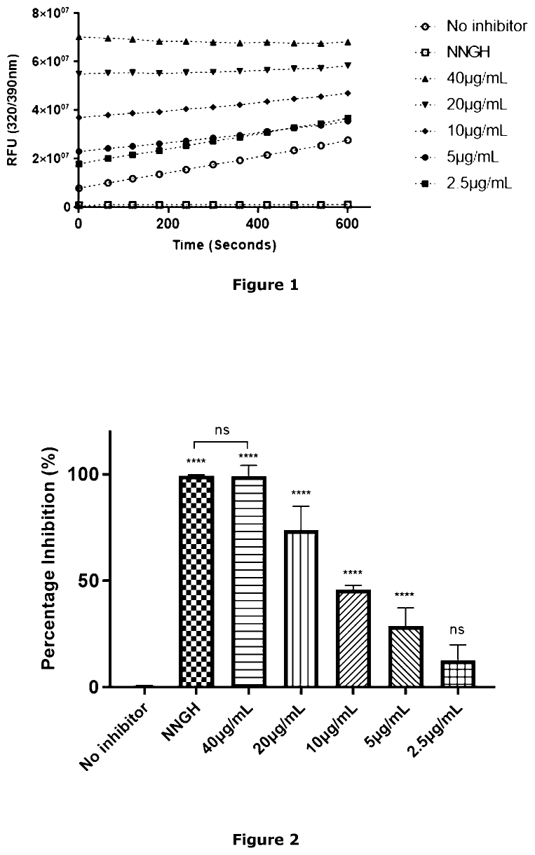

Synthesised 3,6,7-trimethyllumazine was supplemented into the reaction mix at the concentrations found in Manuka honey from 2.5-40 μg/ml (3-44 μg/ml measured by LC-MS/MS). As shown in FIG. 1, the change in fluorescence intensities were linear for all 3,6,7-trimethyllumazine samples and controls. There was almost no change in fluorescence in the NNGH positive control. In contrast, a steady increase in fluorescence was observed for the negative control without inhibitor. 3,6,7-trimethyllumazine samples displayed higher initial fluorescence due to its autofluorescence nature. A fluorescence control was included in this assay for each 3,6,7-trimethyllumazine concentration.

In this study, 3,6,7-trimethyllumazine at all tested concentrations exhibited inhibitory activities on MMP-9 ranging between 12% to 99%, as shown in FIG. 2. In comparison with the negative control with no inhibitor, MMP-9 activities were significantly inhibited by 3,6,7-trimethyllumazine at concentrations higher or equal to 5 μg/ml (All p<0.05). 3,6,7-trimethyllumazine at 2.5 μg/ml inhibited MMP-9 activity by 12%, but the inhibition was not significant (p>0.05). 3,6,7-trimethyllumazine almost completely inhibited MMP-9 at 40 μg/ml, there was no significant difference compared to the NNGH control (p>0.05). The inhibition of MMP-9 appeared to be dose-dependent on 3,6,7-trimethyllumazine concentration, as higher 3,6,7-trimethyllumazine concentration always displayed stronger inhibition comparing to the lower concentrations (all p<0.05). Data are shown as mean±SEM. n=4. ****p<0.0001.

The percentage inhibition of MMP-9 positively correlated with the concentration of 3,6,7-trimethyllumazine as shown in FIG. 3. The correlation fits best into a second-order polynomial model with an R² of 0.9965. Based on these data, the IC₅₀ of 3,6,7-trimethyllumazine was calculated as 11.5 μg/ml. The IC₅₀ was calculated as 11.5 μg/ml. Data are shown as mean±SEM. n=4.

### Example 2—Colorimetric Assay

In this example, an MMP-9 colorimetric inhibitor screening kit is used to further investigate the bioactivity of 3,6,7-trimethyllumazine.

### Methods and Materials

The MMP-9 inhibitor screening assay (colorimetric) kits were purchased from Abcam (Melbourne, Australia). The kit contains the recombinant MMP-9 enzyme, MMP inhibitor NNGH, MMP chromogenic substrate, the colorimetric assay buffer and 96-well clear microplate.

The colorimetric kit uses a thiopeptide as a chromogenic substrate (Ac-PLG-[2-mercapto-4-methyl-pentanoyl]-LG-OC2H5), which can be hydrolysed by MMPs to produce a sulfhydryl group. This intermediate product further reacts with DTNB [5,5′-dithiobis(2-nitrobenzoic acid), Ellman's reagent] to form 2-nitro-5-thiobenzoic acid, which can be detected by absorbance at 412 nm. The change in absorbance was measured using the SpectraMax iD3 multi-mode microplate reader (Molecular Devices, San Jose, USA). The assays are performed on a convenient 96-wells microplate with a final reaction volume of 100 μL. Prior to the assay, all testing samples and inhibitor controls were incubated with MMP-9 for 60 min at 37° C. The chromogenic substrate was added into each well to initiate the reaction. The assay was allowed to run for 120 min at 37° C. The absorbance was measured at 1 min intervals during the first 20 min, then 10 min intervals till the end of assay.

Recombinant MMP-9 and the chromogenic substrate were used as the positive control to represent 100% enzyme activity. NNGH was used as a negative control. A range of 3,6,7-trimethyllumazine concentrations were diluted with the colorimetric assay buffer to measure the absorbance of the reaction product.

### Results

The underlying inhibitory bioactivity of 3,6,7-trimethyllumazine was further investigated using the MMP-9 colorimetric inhibitor screening kit. The colorimetric kit uses a thiopeptide substrate that can be hydrolysed by MMPs to produce a sulfhydryl group intermediate, which further reacts with Ellman's reagent to from 2-nitro-5-thiobenzoic acid. The Ellman's reagent is used to detect the concentration of protein sulfhydryls, and the reaction product can be detected by absorbance at 412 nm (Riener, Kada, & Gruber, 2002).

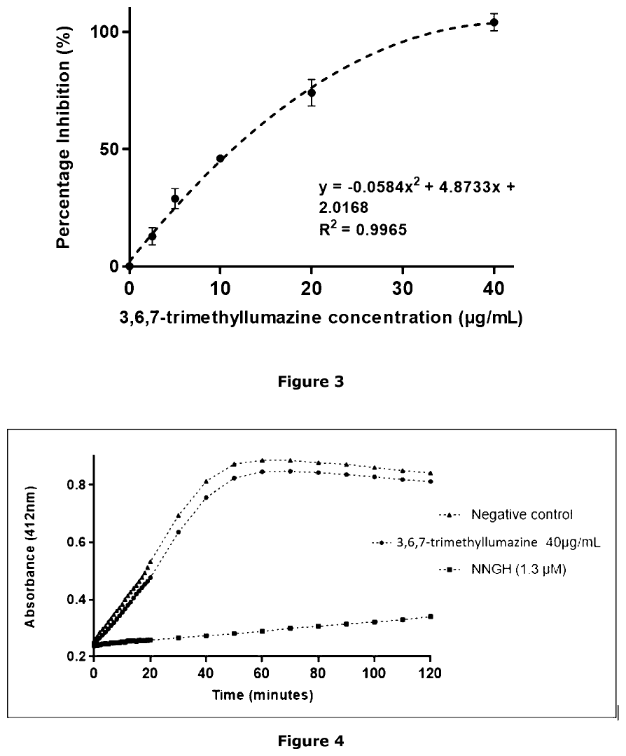

The inhibitory bioactivity was first investigated by supplementing 3,6,7-trimethyllumazine (40 μg/ml) into the reaction mix (FIG. 4). In comparison with the negative control with no inhibitor, the rate of change in absorbance was slightly less in the 3,6,7-trimethyllumazine supplemented sample. The NNGH was employed as the positive control which inhibited most of the MMP-9 activity. NNGH is not expected to completely inhibit MMP-9 at 1.3 μM (Abcam, 2019). The change in absorbance was linear for the 3,6,7-trimethyllumazine sample and controls during the first 40 min. The product appeared to be unstable and begin to breakdown after 40 min. The first 20 min of the reaction was selected for further calculation.

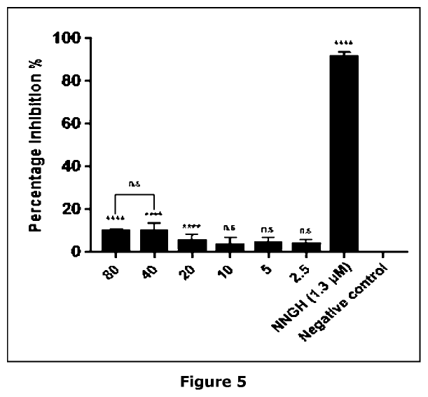

3,6,7-Trimethyllumazine displayed inhibitory bioactivity against MMP-9 at concentrations between 2.5-80 μg/ml. The percentage inhibition was calculated by comparing the absorbance change in 3,6,7-trimethyllumazine samples against the negative control (no inhibitor, 100% MMP-9 activity). As shown in FIG. 5, all 3,6,7-trimethyllumazine samples inhibited MMP-9 by 3.5% to 10% (n=5, 2 reciprocal replicates each). Compared to the negative control, 3,6,7-trimethyllumazine at higher concentrations (20-80 μg/ml) demonstrated significant inhibition on MMP-9 (all p<0.0001). At lower 3,6,7-trimethyllumazine concentrations (2.5-10 μg/ml), the level of MMP-9 inhibition was insignificant (all p>0.05). Increasing 3,6,7-trimethyllumazine concentration from 40 μg/ml to 80 μg/ml did not further inhibit MMP-9 (both 10% inhibition, p>0.05). This suggests that 3,6,7-trimethyllumazine may have a relatively lower binding affinity (Ki) to the MMP-9 enzyme [E] or the enzyme-substrate complex [ES] compared to the chromogenic substrate.

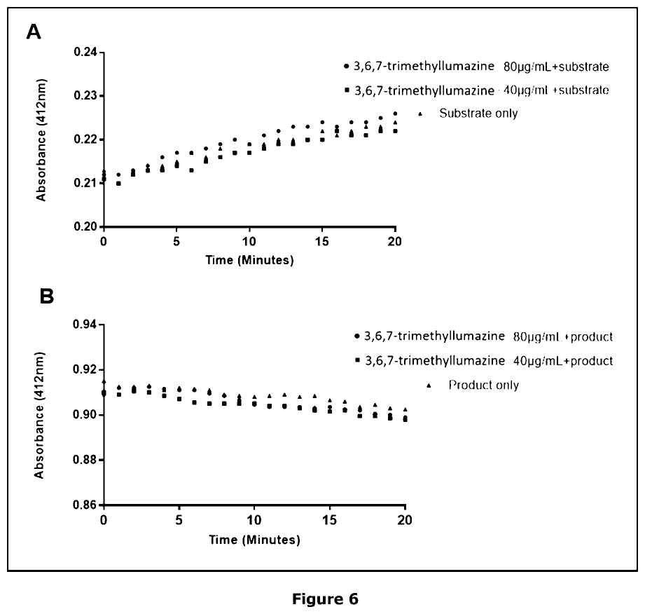

In the absence of MMP-9, 3,6,7-trimethyllumazine did not interfere with the absorbance signal generated by the chromogenic substrate and the reaction product. This was investigated by incubating 3,6,7-trimethyllumazine (40 μg/ml and 80 μg/ml) with the substrate (FIG. 6A) and the reaction product (FIG. 6B) for 20 min. In both cases, 3,6,7-trimethyllumazine did not significantly interfere with the absorbance signal.

### Example 3: Gelatin Gel Zymography

To confirm the inhibition of MMP-9 by 3,6,7-trimethyllumazine the inventors performed Gelatin gel zymography to detect the activity of MMP-9. Gelatin gel zymography is uniquely designed to detect the activity MMP-9 (gelatinase) due to its ability to digest gelatin.

### Methods and Materials

Novex™ 10% Zymogram Plus (Gelatin) Protein Gels (15 wells) were purchased from Thermo Fisher Scientific Inc. (Auckland, New Zealand). All chemicals required for the zymogram analysis were also purchased from Thermo Fisher, these include Novex™ Sharp Pre-stained Protein Standard, Novex Tris-Glycine SDS sample buffer, Novex Tris-Glycine SDS running buffer, Novex Zymogram renaturing buffer and Novex Zymogram developing buffer. Double distilled water was purified from a Sartorius Arium® Pro (18.2 MΩ cm) water purification system. Gelatin gel zymography was performed as an independent technique to confirm the inhibition of MMP-9 from 3,6,7-trimethyllumazine. This technique uses a non-reducing SDS-PAGE (sodium dodecyl sulfate polyacrylamide gel electrophoresis) gel embedded with gelatin. Proteins are migrated and separated during electrophoresis. The SDS is removed after electrophoresis and the gel is then incubated with essential cofactors required for enzymatic activity. The embedded gelatin can be digested by MMP-9, resulting in clear bands on a dark blue background after staining with Coomassie blue dyes. The gelatinase activity is represented by band densitometry, which can be assessed with image analysis software. Gelatin gel zymography is a highly sensitive technique at a relatively low cost (Leber & Balkwill, 1997). Additionally, this approach can simultaneously detect the gelatinase activity of both pro- and active MMPs, as they can be distinguished based on their migration distance through the gel (Rossano et al., 2014).

MMP-9 enzyme was diluted to a final testing concentration of 5 μg/mL. The MMP-9 enzyme was gently mixed with loading buffer and water to achieve a total loading volume of 10 μL per well. Gel electrophoresis was performed using the XCell Surelock™ Mini-Cell system (Thermo Fisher Scientific, Auckland, New Zealand). The upper chamber was filled with 200 mL of 1×Tris-Glycine SDS running buffer, and the lower chamber with 600 mL. The gel was running at a constant voltage of 125V and 30 mA (starting current) for 105 min. After electrophoresis, the gel was removed and incubated in 1× renaturing buffer for 30 min with gentle agitation. Following the incubation, the gel was carefully cut into smaller pieces and further incubated separately in 1× developing buffer or 3,6,7-trimethyllumazine supplemented developing buffer for 30 min under gentle agitation. The gel was further incubated overnight for 13 hours at 37° C. with fresh developing buffer with or without 3,6,7-trimethyllumazine. NNGH were also added into the developing buffer at 2.6 μM as a positive control.

After incubation, the gelatin gel was rinsed with water for three times (5 min each) under gentle agitation. The gel was stained by adding 20 mL of SimplyBlue Safestain and incubated for 2 hours at room temperature under gentle agitation. It was destained by removing the SimplyBlue Safestain and rinsed with water for 2 hours at room temperature under gentle agitation. MMP-9 activities were analysed using densitometry on ImageJ Version 1.52a.

### Results

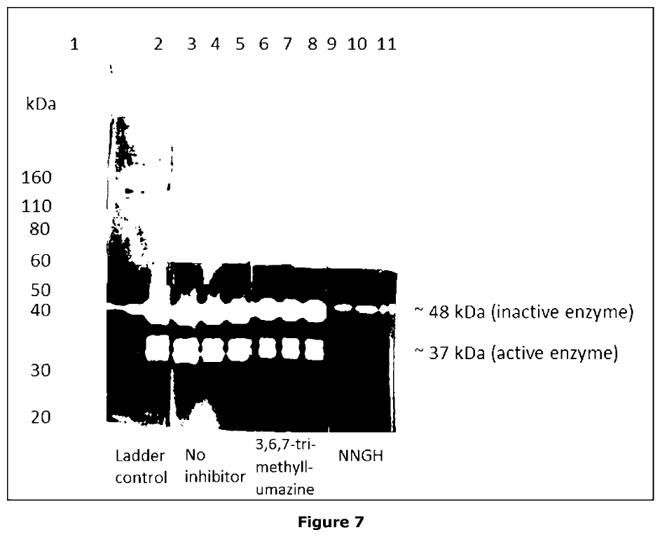

The bioactivity of 3,6,7-trimethyllumazine on MMP-9 was further examined using gelatin zymography, by comparing gelatin gels incubated in normal developing buffer with 3,6,7-trimethyllumazine-supplemented and NNGH-supplemented buffer. The MMP-9 enzyme used in this study were partially activated by 4-aminophenylmercuric acetate (4-APMA) to give more information on molecular interaction. The clear bands on the gel represent gelatinase activity from MMP-9 as shown in FIG. 7. The clear band on top represents gelatinase activity from the fibronectin domain of inactive MMP-9 (˜47 kDa). The bottom band represents the gelatinase activity from active MMP-9, where the pro-domain is cleaved off (˜37 kDa). During electrophoresis, pro-MMP-9 was denatured by SDS, then renatured by removal of SDS with detergents such as Triton X-100 (Ren, Chen, & Khalil, 2017). This refolding process autoactivates a proportion of pro-MMP-9 without cleaving the pro-domain (Woessner, 1995). However, the autoactivated pro-MMP-9 may not represent the true activity in vivo.

3,6,7-Trimethyllumazine appeared to have reduced gelatinase activity from both active and inactive MMP-9 using gelatin zymography. In comparison with the negative control with no inhibitor (FIG. 7, column 3-5), the area of both clear bands appeared to be reduced in 3,6,7-trimethyllumazine-treated gels (FIG. 7, column 6-8). The positive control NNGH completely inhibited the gelatinase activity from active MMP-9 (FIG. 7, column 9-10). There appeared to be some gelatinase activity in NNGH-treated gels from the inactive MMP-9, which is likely a result of the residue gelatinase activity from the fibronectin domain.

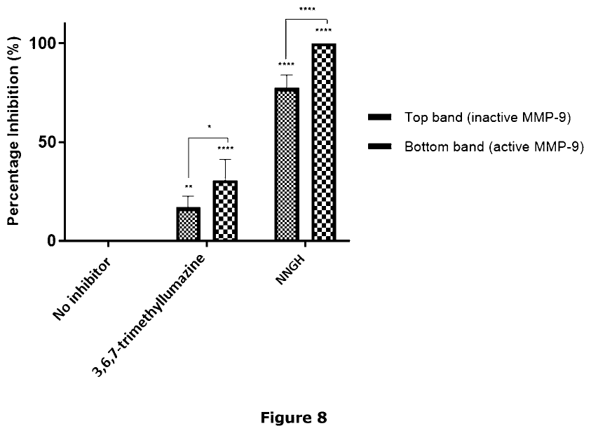

3,6,7-Trimethyllumazine significantly reduced the gelatinase activity from both active and inactive MMP-9 (FIG. 8, both p<0.001). Percentage inhibition from 3,6,7-trimethyllumazine and NNGH were analysed by densitometry and plotted in FIG. 8. Percentage inhibition was calculated by comparing the optical density with the negative control (no inhibitor). As shown, 3,6,7-trimethyllumazine significantly inhibited the activity of active and inactive MMP-9 by 31% and 17%, respectively (both p<0.01). It should be noted that 3,6,7-trimethyllumazine displayed significantly stronger inhibition on the active MMP-9 compared to the inactive MMP-9 (p<0.05). This suggests that 3,6,7-trimethyllumazine is likely to interact more with the zinc active site of MMP-9. The same pattern can also be observed with NNGH treatment, where NNGH specifically interacts with the zinc ion (Bertini et al., 2005).

### Example 4—Molecular Docking of 3,6,7-trimethyllumazine to MMP-9

In this example, a molecular docking study was carried out to predict the non-covalent interactions between 3,6,7-trimethyllumazine and MMP-9.

### Principles

Molecular docking is a computational procedure that attempts to predict non-covalent interaction of ligands with biomacromolecular targets. AutoDock and AutoDock Vina are commonly used computational tools to assist researchers in the determination of biomolecular complexes. The software calculates the minimal interaction energy between targeted protein and ligand while efficiently exploring all torsional freedom. AutoDock is based on an empirical free energy force field and rapid Lamarckian genetic algorithm search method (Goodsell & Olson, 1990; Morris et al., 2009). AutoDock Vina uses a simpler scoring function and rapid gradient-optimisation conformational search, which significantly improves the speed and accuracy (Trott & Olson, 2010).

### Methods and Materials

Molecular docking study on MMP-9 and 3,6,7-trimethyllumazine was carried out using AutoDock Vina v1.1.2. Docking preparation, post-docking analysis and visualisation were performed on Chimera v1.13.1 (Pettersen et al., 2004). The 3D structure of 3,6,7-trimethyllumazine was drawn on Avogadro v1.2.0 (Hanwell et al., 2012). The full 3D crystallographic structure of MMP-9 (PDB ID: 1L6J) was retrieved through the RCSB Protein Data Bank (Elkins et al., 2002).

Docking preparation was performed for both compounds using Chimera. 3,6,7-trimethyllumazine structure was minimised by employing the Smart Minimizer Algorithm. Detection of torsion angles and assignment of Gasteiger charges were also performed on Chimera. The MMP-9 structure was prepared by adding hydrogen atoms, merging non-polar hydrogen atoms, checking missing atom and assign Gasteiger charges. A grid box was defined on the catalytic domain of the MMP-9 enzyme with a volume of Å³=35, 45, 48 (x,y,z coordinates=30, 30, 35). This defines the area of the protein involved in the docking calculation.

Molecular docking was performed on AutoDock Vina with exhaustiveness set as 8 and the number of binding modes as 10. The best binding conformation with the highest score were listed on AutoDock Vina and visualised on Chimera. Potential inter-molecular hydrogen bond for each binding pose was also analysed on Chimera.

### Results

Molecular docking predicted significant binding affinity between 3,6,7-trimethyllumazine and MMP-9. 3,6,7-trimethyllumazine was successfully docked into the active site of MMP-9 with the best docking score of −7.9 using AutoDock Vina (Table 2). The AutoDock Vina score represents the predicted energy required for two compounds to bind, by considering a combination of hydrogen bonds, hydrophobic interactions and torsional penalty (Chang, Ayeni, Breuer, & Torbett, 2010). As a result, the most favourable binding conformation is represented as a negative score. In comparison, the docking score of the most active synthetic MMP-9 inhibitors ranged between −7.6 to −8.9 using AutoDock Vina (Rathee et al., 2018).

TABLE 2
The prediction score calculated by Autodock Vina.

| Score | RMSD l.b. | RMSD u.b. | Number of Hbonds |
|---|---|---|---|
| −7.9 | 0 | 0 | 1 |
| −6.7 | 2.283 | 5.418 | 1 |
| −6.5 | 2.986 | 4.239 | 1 |
| −6.5 | 3.634 | 5.699 | 1 |
| −6.5 | 3.673 | 5.052 | 1 |
| −6.4 | 16.1 | 18.083 | 0 |
| −5.9 | 3.855 | 6.35 | 0 |
| −5.8 | 4.987 | 7.423 | 0 |
| −5.7 | 15.9 | 18.022 | 0 |
| −5.7 | 2.361 | 4.741 | 0 |

3,6,7-Trimethyllumazine was docked into the S′1 substrate binding site by forming a hydrogen bonding with the Tyr⁴²⁰ residue. The S′1 substrate binding site is framed in the centre of the active site cleft closest in proximity to active site zinc. Compared to other binding pockets, the S′1 pocket varies among MMPs in both the amino acid makeup and depth of the pocket (Aureli et al., 2008). As a result, the S′1 pocket determines the substrate binding specificity and is a target for many MMP inhibitors. In particular for MMP-9, co-crystallisation with different inhibitors revealed that the Arg⁴²⁴ residue is highly flexible, which allows some MMP inhibitors to move into the S1′ pocket (Tochowicz et al., 2007).

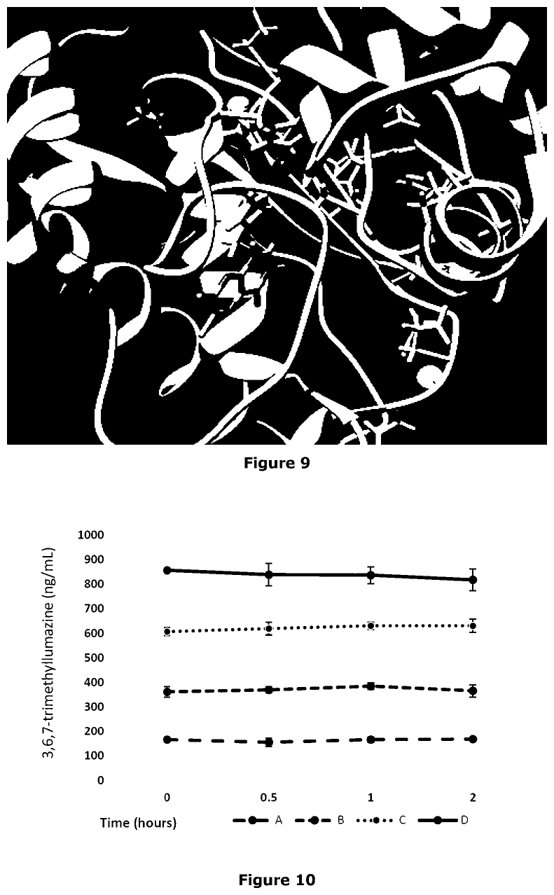

In a previous docking study, synthetic inhibitors with carboxylic acid and sulfonamide hydroxamate group were bound to the S′1 pocket; while the thio-ester group interacts with both S′1 and S1 pocket (Tandon & Sinha, 2011). A potential hydrogen bond was found between the N—H group of 3,6,7-trimethyllumazine and Tyr⁴²⁰ near the wall of the S′1 cavity of MMP9 (FIG. 9). The S′1 wall residues often act as hydrogen acceptors for inter main chain hydrogen bonds to substrates or inhibitors (Tyr⁴²⁰, Pro⁴²¹, Tyr⁴²³) (Tandon & Sinha, 2011). Zinc binding inhibitors with a carbonyl group or N—H groups offer opportunities for hydrogen bonding interactions with the S1′ pocket. (Tandon & Sinha, 2011). Both structures are present in 3,6,7-trimethyllumazine.

These results further supported the binding of 3,6,7-trimethyllumazine at the exosite of MMP-9 located within the fibronectin type II domain. The inventors further identified high gold scores (53.4) of 3,6,7-trimethyllumazine with MMP-9 (Docking was performed with GOLD v5.7.3 with a total of 10 GA runs per ligand and maximum search efficiency. Docked poses were scored with GoldScore). These findings suggested that 3,6,7-trimethyllumazine may interact with the exosite of MMP-9 by disrupting the binding of gelatin. The results from molecular docking analysis further supported the binding of 3,6,7-trimethyllumazine at the exosite of MMP-9 located within the fibronectin type II domain.

### Example 5—Simulation of Gastrointestinal Environment

The extent to which the anti-inflammatory bioactivity of comprising 3,6,7-trimethyllumazine can be retained during the gastrointestinal digestion is unknown. It is possible that this bioactive molecule undergoes modification at low pH or by digestive enzymes, and partially or fully lose its biological activity.

A simulated gastric digestion followed by a simulated intestinal digestion of the 3,6,7-trimethyllumazine-containing honey samples was conducted in vitro. At pre-determined time points during this process, the gastric or intestinal digesta was removed to analyse the remaining amount of 3,6,7-trimethyllumazine.

### Materials and Methods

### Simulation of Gastrointestinal Environment:

The simulated gastrointestinal digestion was carried out using a static model. The simulated gastric fluid (SGF) and the simulated intestinal fluid (SIF) were prepared in accordance with a global consensus protocol (Minekus et al 2014). The SGF has a pH 3 to mimic the fed-state of the stomach. When mixed with Manuka honey (or a honey solution), the final mixture contains 2000 U/mL of pepsin. The SIF has pH 7 to mimic the fed-state of the small intestine, containing 2 mg/mL of pancreatin (8×USP, or based on a protease activity of 200 U/mL) and 20 mM of porcine bile extract before use.

### Gastric Digestion:

In the simulated gastric digestion, 2 g of Manuka honey was incubated in 2 mL of SGF at 37° C. under 95 rpm shaking for a period of 2 h (triplicates). At selected time points (0, 30, 60 and 120 min), a predetermined volume (0.1 mL) of the mixture was withdrawn for 3,6,7-trimethyllumazine analysis. The solution for analysis was kept on ice with an addition of 0.1 mL SIF (pH 7) to stop pepsin activity. As a control group, a pure 3,6,7-trimethyllumazine solution was treated in the same way for comparative purposes.

### Intestinal Digestion:

Following the 2 h gastric digestion, the resulting solution was mixed with SIF (pH 7) at a volume ratio of 1:1, to have a final mixture that contains 1 mg/mL of pancreatin and 10 mM of porcine bile extract. This mixture was incubated at 37° C. under 95 rpm shaking for 4 h (triplicates). At selected time points (0, 60, 120 and 240 min), a predetermined volume (0.1 mL) of the mixture was withdrawn for 3,6,7-trimethyllumazine concentration analysis. The pancreatin activity in the withdrawn solution was quenched by adding 5 mmol/L Pefabloc® (Egger et al 2019).

Analysis of 3,6,7-trimethyllumazine Retention:

The gastric and intestinal digesta for 3,6,7-trimethyllumazine analysis was treated to remove insoluble fractions (e.g. pancreatin) before HPLC analysis. In brief, all samples were diluted with 0.1% formic acid and then centrifuged at 14,000 rpm for 10 min. Supernatant was taken for analysis. The amount of 3,6,7-trimethyllumazine at different time points was analysed using a reverse-phase HPLC system, which has been previously used to analyse 3,6,7-trimethyllumazine and leptosperin as reported in the literature (Bin Lin et al 2017). In brief, the samples were diluted 5 times in 0.1% v/v formic acid. A Hypersil GOLD column (150×2.1 mm, 3 μM particle size) was used as the stationary phase (25° C.), and the mobile phase will consist of 0.1% formic acid (phase A), and 80:20 acetonitrile: 0.1% formic acid (phase B). The injection volume was 3 μL, flow rate 0.200 mL, and a gradient elution as follow was used to separate 3,6,7-trimethyllumazine and others: initial 2 min (5% phase B), at 7 min (25% B), 14 min (50% B), 16 min (100% B), 19 min (5% B) and 20 min (5% B, held 10 min). The signal of 3,6,7-trimethyllumazine was detected at 320 nm.

### Statistical Analysis:

The significance of difference between two mean values was analysed using a two-tailed unpaired Student's t-test. When more than two mean values were compared, significant differences were analysed by a one-way analysis of variance followed by a Bonferroni's multiple comparison test (SPSS Statistics Version 24, IBM). Differences were considered to be statistically significant at p<0.05.

### Results

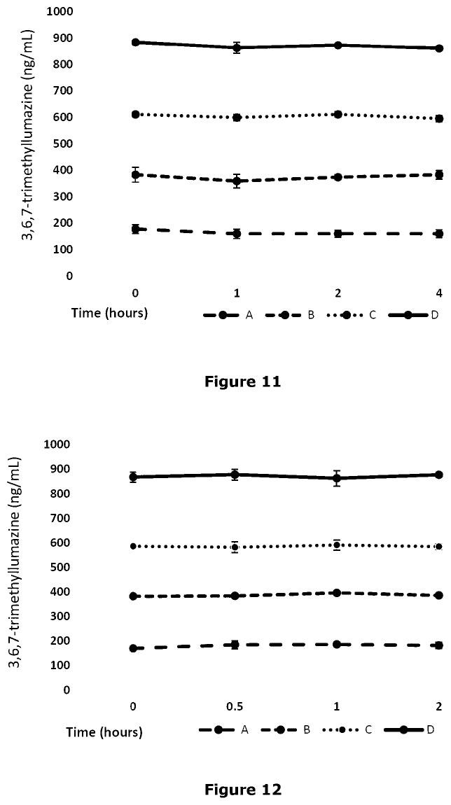

The results from the simulated gastrointestinal digestion of four honey samples indicate that 3,6,7-trimethyllumazine from Manuka honey is highly stable in the harsh environment of the digestive tract. Up to the end of the study, i.e. 2 h gastric digestion plus 4 h intestinal digestion, nearly 100% of the initial 3,6,7-trimethyllumazine amount from the four honey samples could be fully recovered in the digesta. No evident degradation is observed. The detailed dynamics are shown in FIGS. 10 and 11, where in the first 2 h 3,6,7-trimethyllumazine is incubated in simulated gastric fluids, while the subsequent 4 h represents the intestinal digestion stage. Data represent mean±SD, n=3. The raw data are summarised in Table 3.

### Details of Experimental Results

TABLE 3
Table 3: The remaining amount of 3,6,7-trimethyllumazine
during the gastrointestinal digestion of four Manuka honey
samples (A, B, C, D) Data represent mean ± SD, n = 3.

| | Time | | | | |
|---|---|---|---|---|---|
| Phase | (h) | A | B | C | D |
| SGF | 0 | 167 ± 10 | 363 ± 21 | 608 ± 16 | 858 ± 3 |
| | 0.5 | 156 ± 17 | 371 ± 13 | 620 ± 26 | 840 ± 46 |
| | 1 | 167 ± 11 | 386 ± 13 * | 632 ± 15 | 838 ± 34 |
| | 2 | 169 ± 6 | 367 ± 25 | 632 ± 27 | 819 ± 44 |
| SIF | 0 | 179 ± 17 | 384 ± 28 | 612 ± 2 | 885 ± 6 |
| | 1 | 161 ± 18 | 360 ± 26 | 600 ± 12 | 864 ± 21 |
| | 2 | 161 ± 14 | 375 ± 9 | 612 ± 10 | 874 ± 3 |
| | 4 | 161 ± 15 | 384 ± 17 | 596 ± 12 | 862 ± 9 |

* Based on two replicates instead of three due to technical issue.

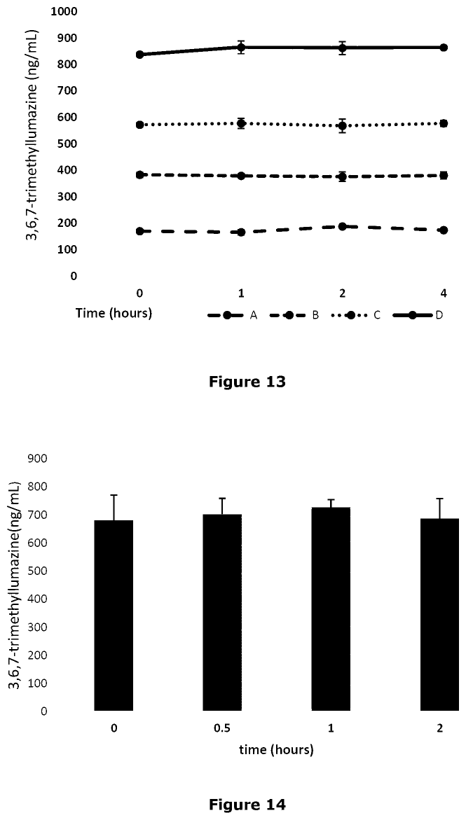

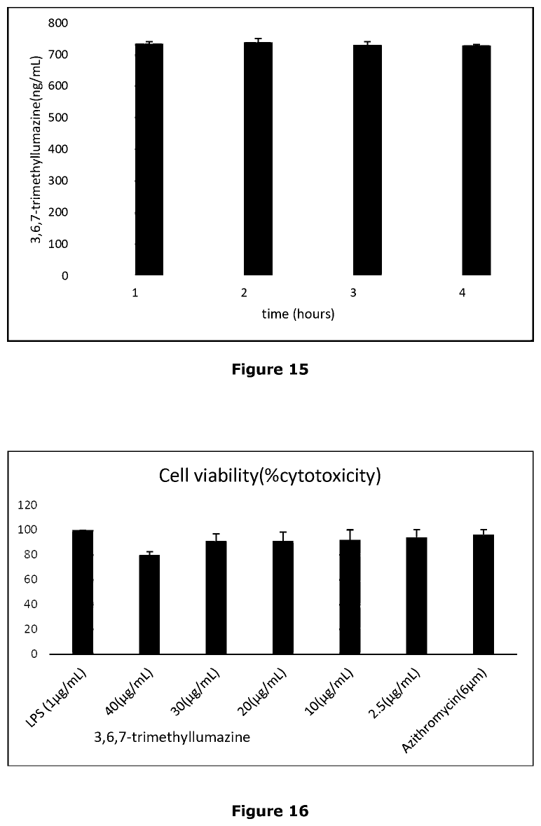

In a subsequent study, the gastrointestinal digestion of diluted Manuka honey samples was conducted to understand the stability of 3,6,7-trimethyllumazine in different concentrations of the Manuka honey. The stability of 3,6,7-trimethyllumazine (pure compound) was also tested using the same in-vitro digestion protocol. The results indicate that the stability of 3,6,7-trimethyllumazine is unchanged, either in 50% (w/w) Manuka honey solutions (FIGS. 12 and 13) or directly exposed to digestive media (Figure. 14 and 15), when compared with the stability profile of non-digested raw honeys. Data represent mean±SD, n=3. No significant degradation is observed. The raw data showing the detailed dynamics are summarised in Table 4.

TABLE 4
Table 4: The remaining amount of 3,6,7-trimethyllumazine
during the gastrointestinal digestion of 50% (w/w) Manuka
honey solutions (A, B, C, D) and 3,6,7-trimethyllumazine
compound. Data represent mean ± SD, n = 3.

| | | | | | | 3,6,7- |
|---|---|---|---|---|---|---|
| | Time | | | | | trimethyl- |
| Phase | (h) | A | B | C | D | lumazine |
| SGF | 0 | 171 ± 9 | 383 ± 2 | 587 ± 6 | 869 ± 21 | 682 ± 90 |
| | 0.5 | 186 ± 16 | 385 ± 1 | 583 ± 22 | 879 ± 22 | 703 ± 57 |
| | 1 | 187 ± 8 | 397 ± 4 | 592 ± 21 | 864 ± 31 | 728 ± 28 |
| | 2 | 183 ± 13 | 387 ± 9 | 586 ± 11 | 878 ± 6 | 688 ± 72 |
| SIF | 0 | 171 ± 3 | 384 ± 10 | 573 ± 9 | 838 ± 5 | 736 ± 6 |
| | 1 | 167 ± 10 | 380 ± 3 | 578 ± 19 | 866 ± 25 | 740 ± 12 |
| | 2 | 189 ± 6 | 377 ± 18 | 569 ± 26 | 863 ± 25 | 732 ± 10 |
| | 4 | 175 ± 7 | 381 ± 13 | 578 ± 12 | 865 ± 1 | 730 ± 3 |

The in-vitro studies on the fate of 3,6,7-trimethyllumazine in the digestive tract clearly indicate high stability of the bio-active compound, 3,6,7-trimethyllumazine, as tested from the, diluted (50% dilution) Manuka honey samples A, B, C and D and in its purest form.

### Example 6—Effect of 3,6,7-trimethyllumazine on Matrix metalloproteinase-9 (MMP-9) in Human Macrophage Cell Lines

The inventors investigated the efficacy of 3,6,7-trimethyllumazine, present in Manuka honey, to inhibit lipopolysaccharides (LPS) induced MMP-9 secretion in human macrophage cell lines (THP-1) using Enzyme Linked Immunosorbent Assay (ELISA) technique.

Macrophages are a potential source of gastric MMPs, as they are known to respond to both bacterial factors and pro-inflammatory cytokines with an increased MMP-9 secretion. Therefore, MMP-9 secretion from THP-1 can be used as a marker of gastric inflammation.

Concentrations between 2.5-40 μg/mL of 3,6,7-trimethyllumazine were tested for MMP-9 inhibitory activity. Azithromycin was selected as a positive control. 3,6,7-trimethyllumazine at 40 μg/mL (38% reduction) and 30 μg/mL (23% reduction) significantly (P<0.05) reduced MMP-9 secretion from the LPS (1 μg/mL) treated differentiated THP-1 cells compared to 20 and 5 μg/mL. 3,6,7-trimethyllumazine at 40 μg/mL reduced MMP-9 and this reduction was slightly higher than Azithromycin over 30 μg/mL. However, based on the cell viability reports, 30 μg/mL (13% cell death) is slightly safer than 40 μg/mL (20% cell death).

### Methods

### Dose:

3,6,7-Trimethyllumazine was tested at doses between 2.5-40 μg/ml for its inhibition of MMP-9 inflammation response using differentiated THP-1 cells.

Cell culture:

THP-1 cells (ATCC, ATCTIB202) were grown in RPMI-1640 (Gibco, 11875093)+0.05 mM 2-mercaptoethanol+10% fetal calf serum (FCS)+1% pen-strep. For experiments, the cells were cultured in RPMI-1640 medium with 10% fetal bovine serum (FBS) only.

THP-1 monocyte cells were seeded at a density of 2.5×10⁵ cells/ml in 96-well plates and differentiated into macrophages using 10 ng/ml of phorbol 12-myristate 13-acetate (PMA) (Bergin et al) (Sigma, P1585-1MG, Lot #SLBX889, 100% purity) for 72 hours. PMA media was then removed from the differentiated THP-1 cells, the cells were then washed once in RPMI-1640 media and then left to rest for 5 hours.

LPS Stimulation and Treatment with 3,6,7-Trimethyllumazine:

The differentiated THP-1 cells were stimulated with LPS from E. coli 055:B5 (Sigma, L6529; Lot #037K4068). LPS tested at a concentration of 1 μg/ml (Kong et al). The cells were incubated with LPS alone or in combination with 3,6,7-trimethyllumazine (received from University of Auckland and diluted in RPMI-1640 at a stock concentration of 1 mg/ml, stock was stored in fridge for 2 days before use) at a concentration range between 2.5-40 μg/ml. 6 μM Azithromycin (Sigma, Cat #75199-25MG, Lot #069M4826V) was used as a positive control (Vandooren et al). The cells were then incubated with the different treatments for 24 hours (Kong et al). After 24 hours, the cell culture media was collected and measured for MMP-9 concentration using MMP-9 ELISA (R&D systems, RDSDY91105 lot #P239459 and DY008 Lot #P239900). The cells were then incubated with WST-1 for cell viability.

Two additional 96-well plates were treated as above, media removed and the plate containing cells were frozen in −80° C. for future RT-PCR experiments.

### Elisa Specificity:

This human MMP-9 assay measures the 92 kDa Pro-MMP-9 and the 82 kDa active MMP-9. It does not measure the 65 kDa form. This assay also recognizes human MMP-9 when complexed to Lipocalin-2/NGAL, isolated from human source material.

The following factors prepared at 50 ng/mL were assayed and exhibited no cross-reactivity or interference: MMP-1, MMP-2, MMP-3, MMP-7, MMP-8, MMP-10, MMP-12, MMP-13, MMP-14, TIMP-2, TIMP-3, TIMP-4, TIMP-4, recombinant mouse MMP-9.

Recombinant human TIMP-1 does not cross-react in this assay but does interfere at concentrations >1.56 ng/ml.

### Cell Viability and Preliminary MMP-9 Secretion Test:

To measure cytotoxicity of 3,6,7-trimethyllumazine at different doses, 2-(4-Iodophenyl)-3-(4-nitrophenyl)-5-(2,4-disulfophenyl)-2H-tetrazolium, monosodium salt (WST-1) (Roche, 11644807001, Lot #45255800) was used. WST-1 is a cell proliferation reagent for measurement of cellular proliferation, viability, and cytotoxicity using a colorimetric assay (Gosert (2011); Peskin (2000)).

After incubation, a portion of the culture media was taken and stored for MMP-9 secretion testing using ELISA. The remaining media in the plates was then removed and 100 μl of WST-1 in RPMI-1640 media (1:10 dilution) was added to each cell well and incubated at 37° C. for a further 4 hours. The plates were then read using a plate reader at wavelength of 450 nm. Cytotoxicity was calculated as follows:

[WST-1 score for each sample/WST-1 score for the control]×100

A WST-1 score below 80% will be considered cytotoxic.

### Statistical Analysis:

In order to better capture the variability, each treatment was done at least in triplicate in each plate (2 plates). Media from the triplicate wells were pooled and analysed in duplicates for the MMP-9 ELISA. A student's test was performed in excel between media with LPS and the different treatments.

### Results

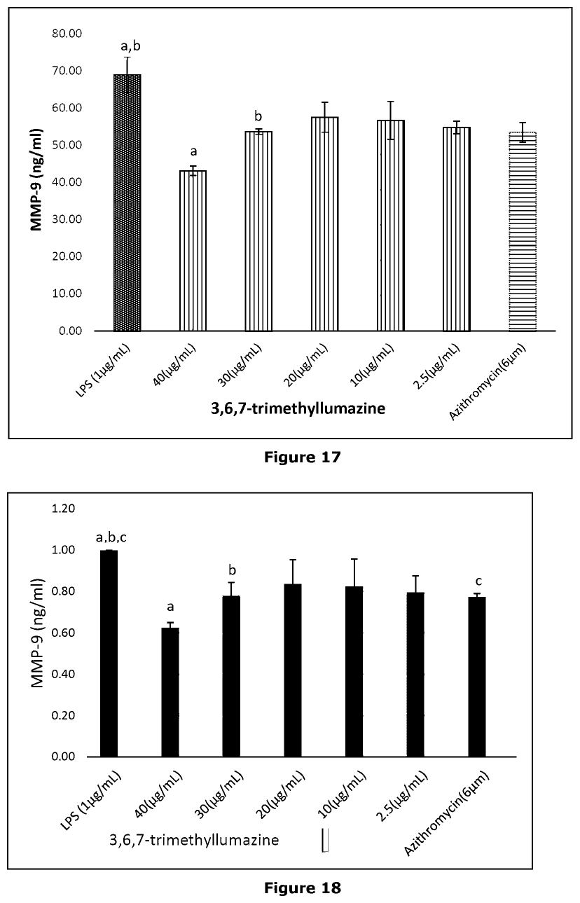

3,6,7-Trimethyllumazine at concentration 40 μg/mL has slightly more % cell toxicity than the other concentrations selected in the study (2.5-30 μg/mL) (FIG. 16). Data is presented as mean±SD. However, it is borderline for consideration (79.8) for it to be toxic. 3,6,7-trimethyllumazine at 40 μg/mL (38% reduction) and 30 μg/mL (23% reduction) significantly (P<0.05) reduced MMP-9 secretion from the LPS (1 μg/mL) treated differentiated THP-1 cells compared to that at 20 and 5 μg/mL (FIG. 17). In FIG. 17 the small letter represents significant differences between the treatments. a—40 μg/mL of 3,6,7-trimethyllumazine inhibits MMP-9 secretion (P=0.02); b—30 μg/mL of 3,6,7-trimethyllumazine inhibits MMP-9 secretion (P=0.02). 3,6,7-trimethyllumazine at 40 μg/mL reduced MMP-9 greater than Azithromycin over 30 μg/mL. Adjusted values (in relation to LPS) are presented in FIG. 18. In FIG. 18 small represents significant differences between the treatments. a—40 μg/mL of 3,6,7-trimethyllumazine inhibits MMP-9 secretion (P=0.00), b—30 μg/mL of 3,6,7-trimethyllumazine inhibits MMP-9 secretion (P=0.04); c—Azithromycin inhibits MMP-9 secretion (P=0.00).

### Example 7: Molecular Docking—TG2, COX-2 and JAK

Molecular docking is a bioinformatic modelling which involves the interaction of two or more molecules to give the stable adduct. It is employed to predict the binding capability of ligands to the targets of interest. Depending upon binding properties of ligand and target, it predicts the three-dimensional structure of any complex. The co-crystallised ligands and additional known inhibitors of the targets (e.g.: TG2, COX-2 and JAK) were docked to validate the pose prediction quality and to serve as positive control for the active ligands. Chemically or pharmacologically related ligands not known to possess e.g. TG2, COX-2 or JAK affinity were included as a negative control. All compounds were minimized with the MMFF94 force-field in Chem3D v18.1. Docking was performed with GOLD v5.7.3 with a total of 10 GA runs per ligand and maximum search efficiency. Docked poses were scored with GoldScore and subsequently rescored with ChemScore.

### Results

### 3,6,7-TRIMETHYLLUMAZINE AS A PUTATIVE JAK-1 LIGAND

Analysis of the score clusters shows very clear differentiation between known actives and decoy ligands. 3,6,7-trimethyllumazine (Gold score—43.31; Chem score—19.33) occupies a space between the two clusters but is closer to the centroid of the decoy ligands than to the centroid of the known actives.

The geometry and electronics of 3,6,7-trimethyllumazine were calculated using a quantum mechanical Hartree-Fock method with the 3-21G basis set. The geometry and electronics analysis on the structure of 3,6,7-trimethyllumazine exhibited three distinct areas of negative electrostatic potential, with a potential hydrogen bond pattern reminiscent of the adenine moiety of adenosine. Without being bound by theory, the inventors predict significant binding of 3,6,7-trimethyllumazine to the ATP binding site of JAK-1. This is due to the structural affinity of 3,6,7-trimethyllumazine for ATP binding sites.

### Structural Data

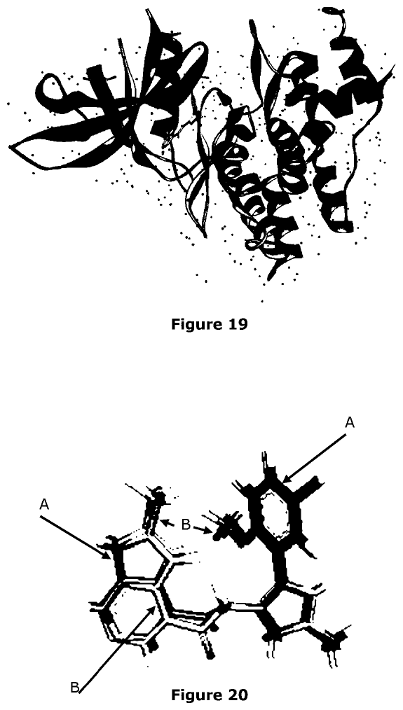

Crystallographically-resolved structures of human JAK1 co-crystallised with various ligands are available on the Protein Data Bank (PDB). Entry 6N7A (FIG. 19) was chosen as the target structure owing to its high resolution (1.33 Å) and chemical similarity of the co-crystallised ligand to 3,6,7-trimethyllumazine.

### Protocol Design

The co-crystallised ligand and additional known JAK1 inhibitors were docked to validate the pose prediction quality and to serve as positive control for active ligands. Co-crystallised ligands from other PDB entries of JAK1 and ligands with JAK1 activity reported in the ChEMBL database were used as the active compound set. Small molecules with similar molecular weight and/or structure were used as decoy ligands. Compounds are either reported as their PDB ID (3 letter code) or CHEMBL ID. All compounds were minimised with the MMFF94 force-field in Chem3D v18.1. Docking was performed with GOLD v5.7.3 with a total of 10 GA runs per ligand and maximum search efficiency. Docked poses were scored with GoldScore and subsequently rescored with ChemScore.

### Docking to Structure 6N7A

Hydrogens were added and the co-crystallised ligand KEV was removed and redocked to validate pose prediction (FIG. 20). The remaining ligands were docked in the same run. The highest-scoring pose was reported except where another pose overlapped more substantially with the coordinates of the co-crystallised ligand (Table 5).

TABLE 5
Scored poses from docking to 6N7A

| Compound | GoldScore | ChemScore |
|---|---|---|
| CHEMBL3645549 | 62.18 | 24.45 |
| KF1 | 58.41 | 23.12 |
| KEP | 58.36 | 17.75 |
| KEV | 57.48 | 26.95 |
| CHEMBL3622836 | 57.00 | 23.47 |
| CHEMBL1553519 | 56.62 | 25.91 |
| KEJ | 55.99 | 20.81 |
| B7V | 55.82 | 18.19 |
| CHEMBL278041 | 55.66 | 26.12 |
| CHEMBL3645544 | 55.56 | 28.89 |
| KEY | 55.48 | 23.13 |
| KF4 | 54.06 | 23.98 |
| 0NH | 53.73 | 20.76 |
| CHEMBL3120960 | 51.27 | 25.12 |
| 9T6 | 49.76 | 21.22 |
| Lepteridine | 43.31 | 19.33 |
| Tramadol | 42.50 | 15.11 |
| Ibuprofen | 38.40 | 11.66 |
| Caffeine | 37.08 | 13.96 |

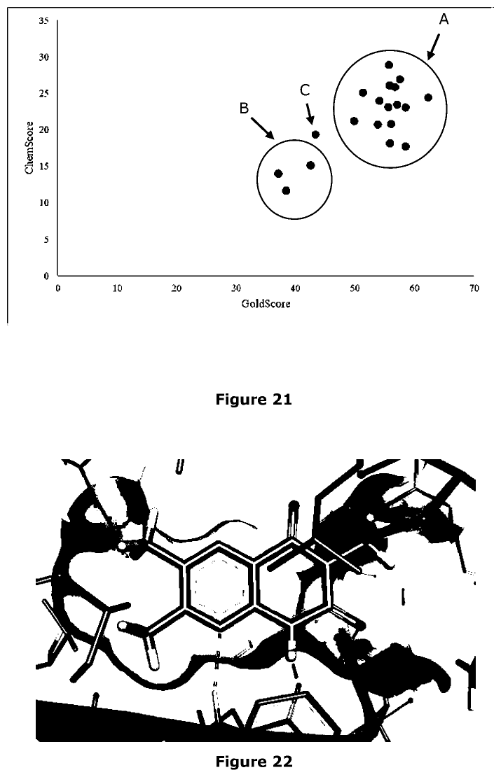

Analysis of the score clusters (FIG. 21) shows very clear differentiation between known actives and decoy ligands. 3,6,7-trimethyllumazine occupies a space between the two clusters of active and decoy ligands. This provides evidence of the potential efficacy of 3,6,7-trimethyllumazine in-vitro.

Analysis of the top ranking pose for 3,6,7-trimethyllumazine shows H bond interactions from three separate ligand atoms to backbone heteroatoms of Leu959 and Ser961. (FIG. 22).

Although the presence of these H bonds is encouraging, the interactions do not involve any amino acid side chains. Backbone interactions are not uncommon in the binding poses of active compounds, but engagement of side chains by H bonding or through aromatic interactions is required to confer specificity to the ligand, as most protein binding sites contain exposed backbone heteroatoms. Given this observation and the results of the cluster analysis 3,6,7-trimethyllumazine has been shown to have highest GoldScore of any of the named JAK-1 actives.

### 3,6,7-TRIMETHYLLUMAZINE AS A PUTATIVE TG2 LIGAND

Analysis of the score clusters shows a curious trend with most of the decoy ligands having relatively high ChemScores, yet low GoldScores. The co-crystallised ligand GDP and ligands from other XRD structures of TG2 have the highest GoldScores but also the lowest ChemScores. 3,6,7-trimethyllumazine has the second lowest GoldScore (37.52) yet a reasonably high ChemScore (20.38).

### Structural Data

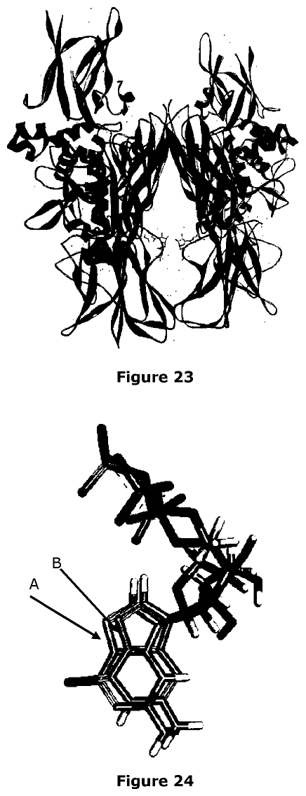

Crystallographically-resolved structures of human transglutaminase 2 co-crystallised with various ligands are available on the Protein Data Bank (PDB). Entry 1KV3 (FIG. 23) was chosen as the target structure as it was the highest resolution (2.8 Å) model of the wild-type available with a small molecule (GDP) bound.

### Protocol Design

The co-crystallised ligand and additional known transglutaminase 2 inhibitors were docked to validate the pose prediction quality and to serve as positive control for active ligands. In addition to other co-crystallised ligands, the ChEMBL database was used to find ligands with favourably low IC₅₀ values to define the active compound set. A set of small molecules with similar chemical structure and/or molecular weight to 3,6,7-trimethyllumazine were used as decoy ligands. All compounds were minimised with the MMFF94 force-field in Chem3D v18.1. Docking was performed with GOLD v5.7.3 with a total of 10 GA runs per ligand and maximum search efficiency. Docked poses were scored with GoldScore and subsequently rescored with ChemScore.

### Docking to Structure 1KV3

Hydrogens were added and the co-crystallised ligand GDP was removed and redocked to validate pose prediction (FIG. 24). The remaining ligands were docked in the same run. The highest-scoring pose was reported except where another pose overlapped more substantially with the coordinates of the co-crystallised ligand (Table 6).

TABLE 6
Scored poses from docking to 1KV3.

| Compound | GoldScore | ChemScore |
|---|---|---|
| GDP | 63.98 | 1.69 |
| 3S3S peptide | 63.37 | 4.22 |
| 3S3J peptide | 56.99 | 5.30 |
| Diclofenac | 52.93 | 14.23 |
| CHEMBL3423195 | 50.18 | 10.09 |
| CHEMBL3423197 | 45.98 | 17.59 |
| CHEMBL2086889 | 45.47 | 14.57 |
| Ibuprofen | 44.73 | 19.58 |
| Mefenamic acid | 44.34 | 20.65 |
| Pentofixylline | 44.08 | 11.32 |
| Caffeine | 43.80 | 11.44 |
| Naproxen | 42.87 | 18.29 |
| CHEMBL3423198 | 42.19 | 18.33 |
| CHEMBL3891796 | 41.62 | 16.32 |
| Toxoflavin | 41.32 | 15.91 |
| Paraxanthine | 40.66 | 14.06 |
| CHEMBL3901025 | 40.41 | 17.89 |
| CHEMBL3968388 | 39.96 | 16.68 |
| Theophylline | 39.50 | 14.66 |
| IBMX | 38.67 | 7.48 |
| Theobromine | 38.02 | 12.12 |
| Lepteridine | 37.52 | 20.38 |
| Tramadol | 36.58 | 22.07 |

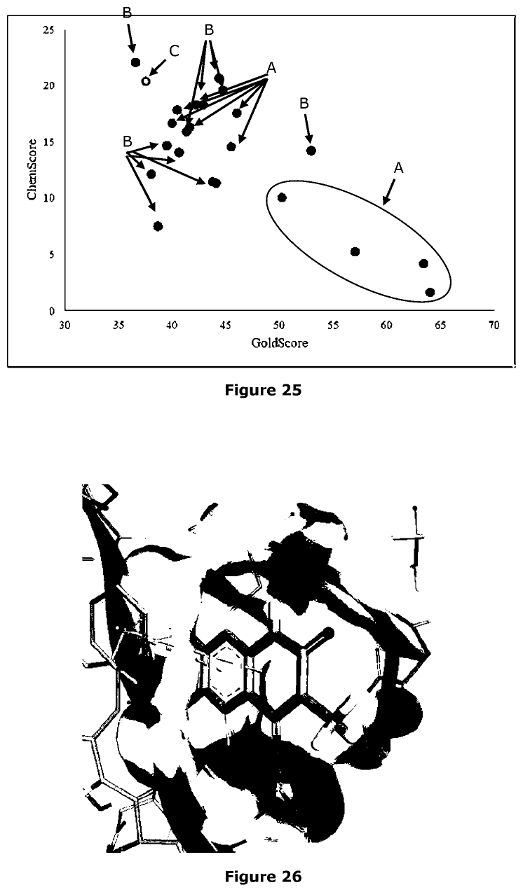

Analysis of the score clusters (FIG. 25) shows a curious trend with most of the decoy ligands having relatively high ChemScores, yet low GoldScores. The co-crystallised ligand GDP and ligands from other XRD structures of transglutaminase 2 have the highest GoldScores but also the lowest ChemScores. 3,6,7-trimethyllumazine has the second lowest GoldScore yet a reasonably high ChemScore.

Analysis of the top ranking pose for 3,6,7-trimethyllumazine shows H bond interactions from Arg580 to a carbonyl and a pyrazine nitrogen (FIG. 26). Additionally, there is a n-n stacking interaction between Phe174 and the pyrazine ring.

However, there is a clash between the H bond donating nitrogen of 3,6,7-trimethyllumazine and an H bond donating backbone nitrogen of the enzyme. Additionally, the surface of the binding pocket is more solvent-exposed than is generally seen, with only the methyl substituents located in a buried cavity. This significantly weakens the strength of the interaction, which is reflected in the relatively low GoldScore (FIG. 25) compared to the other docked ligands. In summary, high ChemScores provides substantial evidence for the efficacy of 3,6,7,-trimethyllumazine in-vitro.

### 3,6,7-TRIMETHYLLUMAZINE AS A PUTATIVE COX-2 LIGAND

The inventors also predict that 3,6,7-trimethyllumazine binds to and inhibits COX-2, and may therefore be useful in treating COX-2 related conditions.

### Example 8: Inhibition of LPS (E coli O111:B4)-Induced COX-2 Protein Expression

We assessed the in vitro activity of 3,6,7-trimethyllumazine to inhibit COX-2 expression in THP-1 human monocytic cells. Monocytic cells, including circulating monocytes, dendritic cells and intestinal resident macrophages, are abundant in the intestinal tract and are known to play a role in a number of COX-2 related conditions, including mediating colitis and gut inflammation.

### Materials and Methods

### Sample Cytotoxicity in THP-1 Cells

Human monocytic THP-1 cells were seeded in 96-well tissue-culture treated plates at a density of 50,000 cells/well in growth media (RPMI media supplemented with 10% fetal bovine serum, 2 mM L-glutamine and penicillin-streptomycin) overnight. 3,6,7-Trimethyllumazine sample dilutions were prepared in phosphate buffered saline (PBS) and cells were treated with either 3,6,7-trimethyllumazine (12.5, 25, 50 and 100 μg/mL), 10 μg/mL dexamethasone, 10 μM indomethacin, or a PBS control before being stimulated with 100 ng/mL LPS (E. coli 0111:B4) for 18 hours. Control wells were treated with hydrogen peroxide (0, 0.5, 1, and 2 mM) for 1 hour prior to addition of WST-1 reagent (Roche, NZ). WST-1 reagent was added to all wells at the end of the 18 hour incubation period, and absorbance readings were subsequently measured 30 min and 1 h and 10 mins after WST-1 addition. Cell viability expressed the percent change from the LPS-stimulated PBS control. Sample doses where cell viability is below 80% of the LPS-stimulated control and are significantly different from the LPS-stimulated or unstimulated control are deemed cytotoxic.

### LPS-Stimulation of THP-1 Monocyte Cells

THP-1 cells were seeded in 12-well tissue-culture treated plates at a density of 500,000 cells/well in growth media overnight. 3,6,7-Trimethyllumazine sample dilutions were prepared in PBS and cells were treated with either 3,6,7-trimethyllumazine (12.5, 25, 50 and 100 μg/mL), 10 μg/mL dexamethasone, 10 μM indomethacin, or a PBS control before being stimulated with 100 ng/mL LPS (E. coli O111:B4) for 24 hours. Because indomethacin is not soluble in aqueous buffer all wells not containing indomethacin had 0.025% DMSO added to control for any DMSO effect on cell viability or activity. Cells were washed with cold PBS and lysed in RIPA buffer containing protease inhibitors (Sigma P2714) for 15 minutes on ice. Debris was pelleted by centrifugation at 14,000×g for 15 min and lysate supernatants were frozen at −80° C. for later analysis by western blot.

### Protein Expression of COX-2

Western blots for semi-quantitative COX-2 (Abcam ab188183) protein expression were conducted on THP-1 monocyte cells lysed with RIPA buffer containing protease inhibitors. A total of 10 μg protein was loaded onto precast 10% acrylamide gels (BioRad, NZ) then separated by electrophoresis at 120 V at room temperature. Proteins were then transferred onto polyvinylidene fluoride (PVDF) membranes by electrophoresis at 90 V for 90 min on ice and was blocked with a commercial buffer (BioRad 12010020) overnight at 4° C. to prevent non-specific protein binding to the membranes. The membranes were then incubated with the primary antibody that correspond to the protein of interest for 1 h. Primary antibodies for COX-2 and p-actin (housekeeping protein; Biolegend 622102) were all sourced from rabbit. After three 10-minute wash cycles with PBS-Tween buffer, membranes were incubated with donkey anti-rabbit IgG (H+L) HRP conjugate (Biolegend 406401) for 1 h. The membranes were then washed as previously described and the bound antibody of interest was detected using an ECL Western Blot substrate kit (BioRad 170-5061). Western blot images were captured with an Amersham™ Imager 600 (GE Healthcare, Chicago, Ill., USA) and densitometry measurement of protein bands was analysed with the accompanying image analysis software.

### Statistical Analysis

For analysis of Western blots, densitometry measurements from the proteins of interest were normalised to the densitometry of p-actin for each sample. A Student's T-test was applied to detect differences in protein expression from THP-1 cells treated with 3,6,7-trimethyllumazine, dexamethasone and indomethacin compared with non-treated LPS-stimulated cells. The significance level was set at p≤0.05.

### Results

### WST-1 Cell Viability Assay

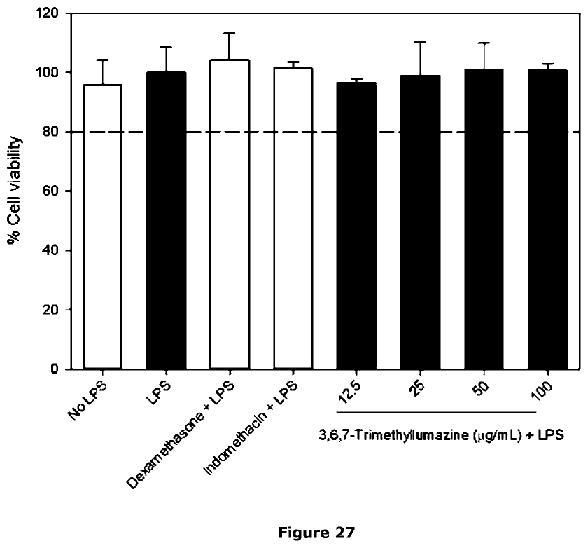

FIG. 27 shows percent cell viability as assessed by the WST-1 assay for THP-1 cells after treatment with PBS, 10 μg/mL Dexamethasone (Dex), 10 μM Indomethacin (Indo) or by Lepteridine™ 3,6,7-Trimethyllumazine (12.5, 25, 50, & 100 μg/mL) and co-stimulation with 1 μg/mL LPS for 18 hours. Data are means±SD. Means below 80% viability (dashed horizontal line) indicate that cells are no longer viable.

The WST-1 cell viability assay for THP-1 cells showed that the concentration of LPS (1 ug/mL and 100 ng/mL respectively), DEX and INDO did not induce cell death. In the THP-1 cells none of the concentrations of 3,6,7-trimethyllumazine induced cell death (FIG. 27). We are confident that any significant reduction in COX-2 protein expression caused by 3,6,7-trimethyllumazine will likely be as a result of interactions with the COX-2 rather than loss of cells.

### Protein Expression of COX-2 in THP-1 Cells

The protein expression of COX-2 in THP-1 monocytes co-treated with LPS with different doses of 3,6,7-trimethyllumazine, dexamethasone or indomethacin were semi-quantitatively measured by Western Blot.

TABLE 7
COX-2 protein expression in THP-1 cells
Table 7: COX-2 protein expression in THP-1 cells following
exposure to LPS and co-treatment with dexamethasone,
indomethacin or 3,6,7-Trimethyllumazine. Data are means ±
SEM from six independent experiments (n = 6).

| | Relative expression |
|---|---|
| Sample | (mean ± SEM) |
| No LPS control | 0.77 ± 0.09 |
| LPS positive control | 1.26 ± 0.11 |

| | | |
|---|---|---|
| Dexamethasone | (10 μg/mL) | 0.85 ± 0.07 |
| Indomethacin | (10 μmol/L) | 1.28 ± 0.20 |

| | |
|---|---|
| 3,6,7-Trimethyllumazine | |
| (μg/mL) | |
| 12.5 | 0.98 ± 0.05 |
| 25 | 0.99 ± 0.07 |
| 50 | 0.90 ± 0.09 |
| 100 | 0.96 ± 0.13 |

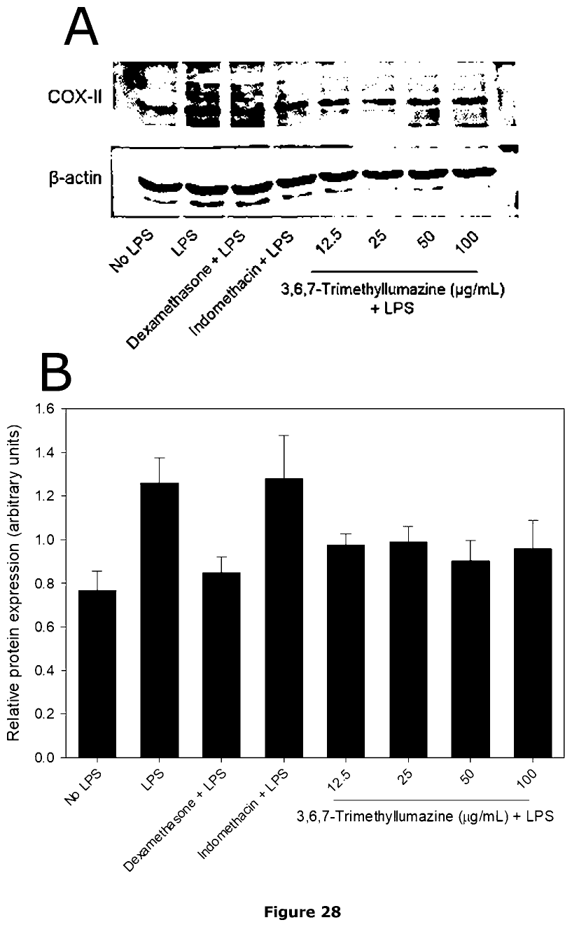

FIG. 28 Protein expression of COX-2 in monocytes following LPS exposure and co-treatment with LPS in combination with dexamethasone, indomethacin or 3,6,7-trimethyllumazine. Representative Western Blots for COX-2 protein expression are presented in A. The relative protein expression of COX-2 in THP-1 cells exposed to the different interventions are presented in B. Data are means±SEM from six independent experiments (n=6).

Exposure of THP-1 monocytes to the concentration of LPS used in this project led to a significant increase in COX-2 protein expression. Co-treatment of THP-1 with LPS and indomethacin (10 μmol/L) did not significantly reduce LPS-induced COX-2 expression. This was expected as indomethacin is a COX-2 inhibitor but is not known in the art to inhibit expression.

The average protein expression of COX-2 in THP-1 cells co-treated with 3,6,7-trimethyllumazine at all the doses tested was reduced compared to the LPS-stimulated cells, indicating the modulatory effect of 3,6,7-trimethyllumazine on LPS-induced COX-2 protein expression. Both dexamethasone and 50 ug/mL 3,6,7-trimethyllumazine showed a significant reduction in COX-2 protein compared to the LPS treatment. 3,6,7-Trimethyllumazine reduced COX-2 protein expression by approx. 16-23% compared to LPS-stimulated cells.

COX-2 protein expression is both positively and negatively regulated, whereby reduction in PGE2 production by COX-2 results in a decrease in COX-2 protein (Cilenti et al., 2021; Inoue et al., 2000). Under dysregulated inflammation high production of PGE2 and other proinflammatory prostaglandins increases COX-2 protein expression and activity (Jabbour et al., 2005; Vichai et al., 2005).

### REFERENCES

Abcam. (2018). MMP9 Inhibitor Screening Assay Kit (Fluorometric) v2a (ab139449). Retrieved from https://www.abcam.com/ps/products/139/ab139449/documents/ab139449 MMP9 Inhibitor Screening Assay Kit (Fluorometric) v2a (website).pdf
Abcam. (2019). MMP9 Inhibitor Screening Assay Kit (Colorimetric) instructions for use (version 4). Retrieved from https://www.abcam.com/ps/products/139/ab139448/documents/ab139448 MMP9 Inhibitor Screening Assay Kit (Colorimetric) v4a (website).pdf
Acuña-Cueva, E. R., Hueso-Ureña, F., Jim\tilde{e}nez-Pulido, S. B. & Moreno-Carretero, M. N. (2000) J. Mol. Model. 6, 433.
Aitken, H. R. M., Johannes, M., Loomes, K. M. & Brimble, M. A. (2013) Tetrahedron Lett. 54, 6916.
Aureli, L., Gioia, M., Cerbara, I., Monaco, S., Fasciglione, G. F., Marini, S., . . . Coletta, M. (2008). Structural bases for substrate and inhibitor recognition by matrix metalloproteinases. Current Medicinal Chemistry, 15, 2192-2222.
Bartke, M. & Pfleiderer, W. (1989) Pteridines. 1, 45.
Bertini, I., Calderone, V., Cosenza, M., Fragai, M., Lee, Y.-M., Luchinat, C., . . . Turano, P. (2005). Conformational variability of matrix metalloproteinases: Beyond a single 3D structure. Proceedings of the National Academy of Sciences of the United States of America, 102, 5334 LP-5339.
B. Lin, K. M. Loomes, G. Prijic, R. Schlothauer, J. M. Stephens, Lepteridine as a unique fluorescent marker for the authentication of Manuka honey, Food Chemistry, 225 (2017) 175-180.
Bergin P J, Anders E, Sicheng W et al. (2004) Increased production of matrix metalloproteinases in Helicobacter pylori-associated human gastritis. Helicobacter 9, 201-210.
Caron, A., Desrosiers, R. R., & Beliveau, R. (2005). Ischemia injury alters endothelial cell properties of kidney cortex: stimulation of MMP-9. Experimental Cell Research, 310, 105-116.
Chang, M. W., Ayeni, C., Breuer, S., & Torbett, B. E. (2010). Virtual screening for HIV protease inhibitors: a comparison of AutoDock 4 and Vina. PloS One, 5, e11955.
Chen, M., Shao, C.-L., Fu, X.-M., Kong, C.-J., She, Z.-G., Wang, C.-Y. (2014) J. Nat. Prod. 77, 1601.
Cilenti, F., Barbiera, G., Caronni, N., Iodice, D., Montaldo, E., Barresi, S., Lusito, E., Cuzzola, V., Vittoria, F. M., Mezzanzanica, L., Miotto, P., Di Lucia, P., Lazarevic, D., Cirillo, D. M., Iannacone, M., Genua, M., & Ostuni, R. (2021). A PGE2-MEF2A axis enables context-dependent control of inflammatory gene expression. Immunity.
Curran, W. V. & Angier, R. B. (1958) J. Am. Chem. Soc. 80, 6095.
Elkins, P. A., Ho, Y. S., Smith, W. W., Janson, C. A., D'Alessio, K. J., McQueney, M. S., Cummings, M. D., Romanic, A. M. (2002) Structure of the C-terminally truncated human ProMMP9, a gelatin-binding matrix metalloproteinase. Acta Crystallogr., Sect. D 58: 1182-1192
Egger L, Ménard O, Baumann C, Duerr D, Schlegel P, Stoll P, et al. Digestion of milk proteins: Comparing static and dynamic in vitro digestion systems with in vivo data. Food Research International. 2019; 118:32-9.
Fingleton B. Matrix metalloproteinases as valid clinical targets. Curr Pharm Des. 2007; 13(3):333-346. doi:10.2174/138161207779313551 Gala, D., DiBenedetto, D., Günter, F., Kugelman, M., Maloney, D., Cordero, M., & Mergelsberg, I. (1997) Org. Process Res. Dev. 1, 85.
Ganguly, K., & Swarnakar, S. (2012). Chronic gastric ulceration causes matrix metalloproteinases-9 and -3 augmentation: Alleviation by melatonin. Biochimie, 94, 2687-2698.
Goodsell, D. S., & Olson, A. J. (1990). Automated docking of substrates to proteins by simulated annealing. Proteins, 8, 195-202.
Gosert R, Rinaldo C H, Wernli M et al. (2011) CMX001 (1-O-hexadecyloxypropyl-cidofovir) inhibits polyomavirus JC replication in human brain progenitor-derived astrocytes. Antimicrobial agents and chemotherapy 55, 2129-2136. Hanwell, M. D., Curtis, D. E., Lonie, D. C., Vandermeersch, T., Zurek, E., & Hutchison, G. R. (2012). Avogadro: an advanced semantic chemical editor, visualization, and analysis platform. Journal of Cheminformatics, 4, 17.
Harris, R. K., Becker, E. D., De Menezes, S. M. C., Granger, P., Hoffman, R. E., Zilm, K. W. (2008) Magn. Reson. Chem. 46, 582.
Inoue, H., Tanabe, T., & Umesono, K. (2000). Feedback control of cyclooxygenase-2 expression through PPARgamma. J Biol Chem, 275, 28028-28032.
Jabbour, H. N., Sales, K. J., Boddy, S. C., Anderson, R. A., & Williams, A. R. (2005). A positive feedback loop that regulates cyclooxygenase-2 expression and prostaglandin F2alpha synthesis via the F-series-prostanoid receptor and extracellular signal-regulated kinase 1/2 signaling pathway. Endocrinology, 146, 4657-4664.
Kakoi, H., Tanino, H., Okada, K., Inoue, S. (1995) Heterocycles. 41, 789.
Kato, Y., Fujinaka, R., Ishisaka, A., Nitta, Y., Kitamoto, N. & Takimoto, Y. (2014) J. Agric. Food. Chem. 62, 6400.
Kato, Y., Umeda, N., Maeda, A., Matsumoto, D., Kitamoto, N., & Kikuzaki, H. (2012) J. Agric. Food. Chem. 60, 3418.
Kong C-S, Kim J-A, Ahn B-N et al. (2011) Potential effect of phloroglucinol derivatives from Ecklonia cava on matrix metalloproteinase expression and the inflammatory profile in lipopolysaccharide-stimulated human THP-1 macrophages. Fisheries Science 77, 867-873.
Leber, T. M., & Balkwill, F. R. (1997). Zymography: a single-step staining method for quantitation of proteolytic activity on substrate gels. Analytical Biochemistry, 249, 24-28.
Lempinen, M., Inkinen, K., Wolff, H., & Ahonen, J. (2000). Matrix Metalloproteinases 2 and 9 in Indomethacin-Induced Rat Gastric Ulcer. European Surgical Research, 32(3), 169-176.
Li, S.-L., Zhao, J.-R., Ren, X.-Y., Xie, J.-P., Ma, Q.-Z., & Rong, Q.-H. (2013). Increased expression of matrix metalloproteinase-9 associated with gastric ulcer recurrence. World Journal of Gastroenterology, 19, 4590-4595.
Luisa Minghetti., Role of COX-2 in Inflammatory and Degenerative Brain Diseases Inflammation in the Pathogenesis of Chronic Diseases, 2007, Volume 42., ISBN 978-1-4020-5687-1
Manicone, A. M., McGuire, J. K. (2008). “Matrix metalloproteinases as modulators of inflammation.” Seminars in Cell & Developmental Biology 19(1): 34-41.
Meidinger, S. (2013) Medical honey and chronic wound healing—Are there honey compounds that inhibit matrix metalloproteinases?, thesis in partial fulfilment of the requirements of the University of Auckland for the degree of BSc Honours in Biomedical Science, unpublished
Meyer, S. W., Mordhorst, T. F., Lee, C., Jensen, P. R., Fenical, W., Kock, M. (2010) Org. Biomol. Chem. 8, 2158.
M. Minekus, M. Alminger, P. Alvito, S. Ballance, T. Bohn, C. Bourlieu, F. Carriere, R. Boutrou, M. Corredig, D. Dupont, C. Dufour, L. Egger, M. Golding, S. Karakaya, B. Kirkhus, S. Le Feunteun, U. Lesmes, A. Macierzanka, A. Mackie, S. Marze, D. J. McClements, O. Menard, I. Recio, C. N. Santos, R. P. Singh, G. E. Vegarud, M. S. Wickham, W. Weitschies, A. Brodkorb, A standardised static in vitro digestion method suitable for food—an international consensus, Food Funct, 5 (2014) 1113-1124.
Minghetti, L, Cyclooxygenase-2 (COX-2) in Inflammatory and Degenerative Brain Diseases, Journal of Neuropathology & Experimental Neurology, Volume 63, Issue 9, September 2004, Pages 901-910
Morris, G. M., Huey, R., Lindstrom, W., Sanner, M. F., Belew, R. K., Goodsell, D. S., & Olson, A. J. (2009). AutoDock4 and AutoDockTools4: Automated docking with selective receptor flexibility. Journal of Computational Chemistry, 30, 2785-2791.
Pettersen, E. F., Goddard, T. D., Huang, C. C., Couch, G. S., Greenblatt, D. M., Meng, E. C., & Ferrin, T. E. (2004). UCSF Chimera—a visualization system for exploratory research and analysis. Journal of Computational Chemistry, 25, 1605-1612.
Peskin A V, Winterbourn C C (2000) A microtiter plate assay for superoxide dismutase using a water-soluble tetrazolium salt (WST-1). Clinica chimica acta 293, 157-166.
Pfleiderer, W. & Fink, H. (1963) Chem. Ber. 96, 2950.
Pfleiderer, W. & Hutzenlaub, W. (1973) Chem. Ber. 106, 3149.
Pfleiderer, W. (1984) Tetrahedron Lett. 25, 1031.
Pradeepkumar Singh, L., Kundu, P., Ganguly, K., Mishra, A., & Swarnakar, S. (2007). Novel role of famotidine in downregulation of matrix metalloproteinase-9 during protection of ethanol-induced acute gastric ulcer. Free Radical Biology and Medicine, 43(2), 289-299. Retrieved from https://doi.org/10.1016/j.freeradbiomed.2007.04.027
Ram, V. J., Knappe, W. R. & Pfleiderer, W. (1977) Tetrahedron Lett. 18, 3795.
Ram, V. J., Knappe, W. R. & Pfleiderer, W. (1982) Liebigs Ann. Chem. 1982, 762.
Rathee, D., Lather, V., Grewal, A. S., & Dureja, H. (2018). Targeting matrix metalloproteinases with novel diazepine substituted cinnamic acid derivatives: design, synthesis, in vitro and in silico studies. Chemistry Central Journal, 12, 41.
Rybakowski J. K. (2009). Matrix Metalloproteinase-9 (MMP9)-A Mediating Enzyme in Cardiovascular Disease, Cancer, and Neuropsychiatric Disorders. Cardiovascular psychiatry and neurology, 2009, 904836.
Reijerkerk, A., Kooij, G., van der Pol, S. M. A., Khazen, S., Dijkstra, C. D., & de Vries, H. E. (2006). Diapedesis of monocytes is associated with MMP-mediated occludin disappearance in brain endothelial cells. The FASEB Journal: Official Publication of the Federation of American Societies for Experimental Biology, 20, 2550-2552.
Ren, Z., Chen, J., & Khalil, R. A. (2017). Zymography as a Research Tool in the Study of Matrix Metalloproteinase Inhibitors. Methods in MolecularBiology (Clifton, N.J.), 1626, 79-102.
Riener, C. K., Kada, G., & Gruber, H. J. (2002). Quick measurement of protein sulfhydryls with Ellman's reagent and with 4,4′-dithiodipyridine. Analytical and Bioanalytical Chemistry, 373, 266-276.
Rein M J, Renouf M, Cruz-Hernandez C, Actis-Goretta L, Thakkar S K, da Silva Pinto M. Bioavailability of bioactive food compounds: a challenging journey to bioefficacy. Br J Clin Pharmacol. 2013; 75(3):588-602. doi:10.1111/j.1365-2125.2012.04425.x
Reinhard, S. M., Razak, K., & Ethell, I. M. (2015). A delicate balance: role of MMP-9 in brain development and pathophysiology of neurodevelopmental disorders. Frontiers in cellular neuroscience, 9, 280. https://doi.org/10.3389/fncel.2015.00280 Ritzmann, G. & Pfleiderer, W. (1973) Chem. Ber. 106, 1401.
Rossano, R., Larocca, M., Riviello, L., Coniglio, M. G., Vandooren, J., Liuzzi, G. M., . . . Riccio, P. (2014). Heterogeneity of serum gelatinases MMP-2 and MMP-9 isoforms and charge variants. Journal of Cellular and Molecular Medicine, 18, 242-252.
Schonbeck, U., Mach, F., & Libby, P. (1998). Generation of biologically active IL-1 beta by matrix metalloproteinases: a novel caspase-1-independent pathway of IL-1 beta processing. Journal of Immunology, 161, 3340-3346.
Seif, F., Khoshmirsafa, M., Aazami, H., Mohsenzadegan, M., Sedighi, G., & Bahar, M. (2017). The role of JAK-STAT signaling pathway and its regulators in the fate of T helper cells. Cell Commun Signal, 15, 23.
Singh, L. P., Kundu, P., Ganguly, K., Mishra, A., & Swarnakar, S. (2007). Novel role of famotidine in downregulation of matrix metalloproteinase-9 during protection of ethanol-induced acute gastric ulcer. Free Radical Biology and Medicine, 43, 289-299.
Southon, I. W. &. Pfleiderer, W. (1978) Chem. Ber. 111, 971.
Stallmach, A., Chan, C. C., Ecker, K-W., Feifel, G., Herbst, H., Schuppan, D., Zeitz, M. (2000). “Comparable expression of matrix metalloproteinases 1 and 2 in pouchitis and ulcerative colitis.” Gut 47(3): 415-422.
Swarnakar, S., Ganguly, K., Kundu, P., Banerjee, A., Maity, P., & Sharma, A. V. (2005). Curcumin regulates expression and activity of matrix metalloproteinases 9 and 2 during prevention and healing of indomethacin-induced gastric ulcer. Journal of Biological Chemistry, 280(10), 9409-9415. Retrieved from https://doi.org/10.1074/jbc.M413398200
Swarnakar, S., Mishra, A., Ganguly, K., & Sharma, A. V. (2007). Matrix metalloproteinase-9 activity and expression is reduced by melatonin during prevention of ethanol-induced gastric ulcer in mice. Journal of Pineal Research, 43, 56-64.
Tandon, A., & Sinha, S. (2011). Structural insights into the binding of MMP9 inhibitors. Bioinformation, 5, 310-314.
Tochowicz, A., Maskos, K., Huber, R., Oltenfreiter, R., Dive, V., Yiotakis, A., . . . Goettig, P. (2007). Crystal structures of MMP-9 complexes with five inhibitors: contribution of the flexible Arg424 side-chain to selectivity. Journal of Molecular Biology, 371, 989-1006.
Trott, O., & Olson, A. J. (2010). AutoDock Vina: improving the speed and accuracy of docking with a new scoring function, efficient optimization, and multithreading. Journal of Computational Chemistry, 31, 455-461.
Uhlmann, E. & Pfleiderer, W. (1981) Heterocycles. 15, 437.
Van den Steen, P. E., Proost, P., Wuyts, A., Van Damme, J., & Opdenakker, G. (2000). Neutrophil gelatinase B potentiates interleukin-8 tenfold by aminoterminal processing, whereas it degrades CTAP-III, PF-4, and GRO-alpha and leaves RANTES and MCP-2 intact. Blood, 96, 2673-2681.
Vandooren J, Knoops S, Buzzo J L A et al. (2017) Differential inhibition of activity, activation and gene expression of MMP-9 in THP-1 cells by azithromycin and minocycline versus bortezomib: A comparative study. PloS one 12.
Vichai, V., Suyarnsesthakorn, C., Pittayakhajonwut, D., Sriklung, K., & Kirtikara, K. (2005). Positive feedback regulation of COX-2 expression by prostaglandin metabolites. Inflamm Res, 54, 163-172.
Vindigni S M, Zisman T L, Suskind D L, Damman C J. The intestinal microbiome, barrier function, and immune system in inflammatory bowel disease: a tripartite pathophysiological circuit with implications for new therapeutic directions. Therap Adv Gastroenterol. 2016; 9(4):606-625. doi:10.1177/1756283X16644242 Voerman, G., Cavalli, S., van der Marel, G. A., Pfleiderer, W., van Boom, J. H., & Filippov, D. V. (2005) J. Nat. Prod. 68, 938.
Wang, D., DuBois, R. The role of COX-2 in intestinal inflammation and colorectal cancer. Oncogene 29, 781-788 (2010). https://doi.org/10.1038/onc.2009.421
Wang, W., Li, F., Xu, Y., Wei, J., Zhang, Y., Yang, H., Gao, B., Yu, G., & Fang, D. (2018). JAK1-mediated Sirt1 phosphorylation functions as a negative feedback of the JAK1-STAT3 pathway. J Biol Chem, 293, 11067-11075.
Woessner, J. F. (1995). Quantification of matrix metalloproteinases in tissue samples. Methods in Enzymology.

1. A method of preventing, ameliorating or treating a COX-2 associated condition in a subject, comprising administering to a subject in need thereof a composition comprising 3,6,7-trimethyllumazine.

2. The method of claim 1 wherein the COX-2 associated condition is an inflammatory condition.

3. The method of claim 2 wherein the inflammatory condition is associated with inflammation of the gastrointestinal tract.

4. The method of claim 1 wherein the COX-2 associated condition is selected from the group consisting of; gastrointestinal inflammatory diseases, gastric ulcers, peptic ulcers, gastritis, inflammatory bowel disease (IBD), Crohn's disease, ulcerative colitis, irritable bowel syndrome (IBS), digestive disease, gastroesophageal reflux disease (GERD), heartburn, acid reflux, Helicobacter pylori infection, mouth ulcers, stomatitis, pharyngitis, gingivitis, esophageal ulcers, inflammatory and degenerative nervous system disorders, neuropsychiatric illnesses, schizophrenia, bipolar mood disorder, neurodegenerative disorders, traumatic brain injury, multiple sclerosis, Alzheimer's disease, nervous system disorders, Parkinson's disease, seizures, brain hypoxia/ischemia, Creutzfeldt-Jakob disease, amyotrophic lateral sclerosis, arthritis, rheumatoid arthritis, juvenile rheumatoid arthritis, ankylosing spondylitis, chronic inflammation, cardiovascular diseases, cancer, pain, colorectal cancer (CRC) and musculoskeletal diseases.

5. The method of claim 1 wherein the COX-2 associated condition is pain.

6. The method of claim 5 wherein the pain is acute pain, chronic pain and/or dysmenorrhea.

7. A method of preventing, ameliorating or treating COX-2 associated inflammation in a subject comprising administering to a subject in need thereof a composition comprising 3,6,7-trimethyllumazine.

8. The method of claim 7 wherein the inflammation is associated with the gastrointestinal tract of a subject.

9. A method of preventing, ameliorating or treating COX-2 associated pain in a subject comprising administering to a subject in need thereof a composition comprising 3,6,7-trimethyllumazine.

10. The method of claim 9 wherein the pain is acute pain, chronic pain and/or dysmenorrhea.

11. The method of any one of the preceding claims wherein the origin of the 3,6,7-trimethyllumazine is honey.

12. The method of claim 11 wherein the honey is of a floral origin substantially from: Leptospermum scoparium, Leptospermum polygalifolium, Leptospermum subtenue, and/or combinations thereof.

13. The method of any one of claims 1 to 10 wherein the origin of the 3,6,7-trimethyllumazine is nectar, roots, fruit, seeds, bark, oil, leaves, wood, stems or other plant material from Leptospermum.

14. The method of claim 13 wherein the origin of the 3,6,7-trimethyllumazine is nectar, roots, fruit, seeds, bark, oil, leaves, wood, stems or other plant material from a plant selected from: Leptospermum scoparium, Leptospermum polygalifolium, Leptospermum subtenue, and/or combinations thereof.

15. The method of any one of claims 1 to 10 wherein the 3,6,7-trimethyllumazine is synthetic.

16. The method of any one of claims 1 to 10 wherein the composition comprising 3,6,7-trimethyllumazine comprises honey or a honey extract.

17. The method of any one of the preceding claims wherein the composition comprises a therapeutically effective amount of 3,6,7-trimethyllumazine.

18. The method of any one of the preceding claims wherein the composition comprises about 2.5 μg/mL to about 1000 μg/mL 3,6,7-trimethyllumazine.

19. The method of any one of the preceding claims wherein the composition comprises about 2.5 μg/mL to about 80 μg/mL 3,6,7-trimethyllumazine.

20. The method of any one of the preceding claims wherein the composition comprises about 2.5 μg/mL, about 5 μg/mL, about 10 μg/mL, about 20 μg/mL, about 40 μg/mL, about 50 μg/mL, about 60 μg/mL, about 70 μg/mL or about 80 μg/mL 3,6,7-trimethyllumazine.

21. The method of any one of claims 1 to 17 wherein the composition comprises about 5 mg/kg to about 3000 mg/kg 3,6,7-trimethyllumazine.

22. The method of any one of claims 1 to 17 and 21 wherein the composition comprises about 5 mg/kg to about 80 mg/kg 3,6,7-trimethyllumazine.

23. The method of claim 22 wherein the composition comprises about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 70 mg/kg or about 80 mg/kg of 3,6,7-trimethyllumazine.

24. The method of any one of the preceding claims wherein the composition comprising 3,6,7-trimethyllumazine is formulated as a liquid formulation, fast moving consumer good, capsule, tablet, chewable tablet, gel, lotion, powder, suppository, cosmetic formulation, intravenous preparation, intramuscular preparation, subcutaneous preparation, solution, food, beverage, dietary supplement or spray.

25. The method of any one of the preceding claims wherein the composition comprising 3,6,7-trimethyllumazine has a standardised concentration of 3,6,7-trimethyllumazine obtained by:
selecting a first composition with a known concentration of 3,6,7-trimethyllumazine;
selecting at least one further composition with a known concentration of 3,6,7-trimethyllumazine; and
combining the first composition with the second composition to obtain a composition with a standardised 3,6,7-trimethyllumazine concentration of about 5 mg/kg to about 3000 mg/kg.

26. The method of any one of claims 1 to 24 wherein the composition comprising 3,6,7-trimethyllumazine has a standardised concentration of 3,6,7-trimethyllumazine obtained by:
selecting a first composition with a known concentration of 3,6,7-trimethyllumazine; and
combining the selected first composition with one or more of:
synthetic 3,6,7-trimethyllumazine;
isolated 3,6,7-trimethyllumazine;
a honey extract comprising 3,6,7-trimethyllumazine; and/or
3,6,7-trimethyllumazine derived directly from a plant of the genus Leptospermum;
to form a composition with a standardised 3,6,7-trimethyllumazine concentration of about 5 mg/kg to about 3000 mg/kg.

27. A method of making a composition with anti-inflammatory, analgesic and/or COX-2 inhibitory activity comprising:
a. testing a first composition comprising honey for 3,6,7-trimethyllumazine concentration;
b. testing at least one further composition comprising honey for 3,6,7-trimethyllumazine concentration;
c. selecting a composition comprising honey with a 3,6,7-trimethyllumazine concentration greater than about 5 mg/kg;
d. selecting at least one further composition comprising honey with a 3,6,7-trimethyllumazine concentration greater than about 5 mg/kg 3,6,7-trimethyllumazine; and
e. combining the selected compositions comprising honey to form a honey composition with a 3,6,7-trimethyllumazine concentration of about 5 mg/kg to about 80 mg/kg.

28. The method of claim 27 wherein a composition comprising honey is selected if it has a concentration of 3,6,7-trimethyllumazine greater than about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 70 mg/kg or about 80 mg/kg.

29. A method of identifying a composition as having anti-inflammatory, analgesic and/or COX-2 inhibitory activity comprising:
a. testing a composition for 3,6,7-trimethyllumazine concentration; and
i. identifying the composition as having anti-inflammatory, analgesic and/or COX-2 inhibitory activity if it contains a 3,6,7-trimethyllumazine concentration greater than about 5 mg/kg; or
ii. identifying the composition as not having anti-inflammatory, analgesic and/or COX-2 inhibitory activity if it contains a 3,6,7-trimethyllumazine concentration lower than about 5 mg/kg.

30. The method of claim 29 wherein the composition comprises honey.

31. A composition comprising 3,6,7-trimethyllumazine for use in any one of the methods as claimed in claims 1 to 26.

32. Use of a composition comprising 3,6,7-trimethyllumazine in the manufacture of a medicament for preventing, ameliorating or treating a COX-2 associated condition.

33. Use of a composition comprising 3,6,7-trimethyllumazine in the manufacture of a medicament for preventing, ameliorating or treating COX-2 associated inflammation.

34. Use of a composition comprising 3,6,7-trimethyllumazine in the manufacture of a medicament for preventing, ameliorating or treating COX-2 associated pain.

35. A method of preventing, ameliorating or treating a TG2 and/or JAK associated condition in a subject, comprising administering to a subject in need thereof a composition comprising 3,6,7-trimethyllumazine.

36. Use of a composition comprising 3,6,7-trimethyllumazine in the manufacture of a medicament for preventing, ameliorating or treating a TG2 and/or JAK associated condition.
